SEQUENCE LISTING (91 sequences)

SEQ ID NO: 1 - protein, 42 aa, synthetic construct
KRGRKKLLYIFKQPFMRPVQTTQEEDGCSCRFPEEEEGGCEL

SEQ ID NO: 2 - DNA, 126 bp, synthetic construct
  source 1..126: mol_type = other DNA
aaacggggcagaaagaaactcctgtatatattcaaacaaccatttatgagaccagtacaaactactcaagaggaagacggctgtagctgccgatttccagaagaagaagaaggaggatgtgaactg

SEQ ID NO: 3 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 4 - protein, 41 aa, synthetic construct
RSKRSRLLHSDYMNMTPRRPGPTRKHYQPYAPPRDFAAYRS

SEQ ID NO: 5 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 6 - protein, 41 aa, synthetic construct
RSKRSRLLHSDYMFMTPRRPGPTRKHYQPYAPPRDFAAYRS

SEQ ID NO: 7 - DNA, 123 bp, synthetic construct
  source 1..123: mol_type = other DNA
aggagtaagaggagcaggctcctgcacagtgactacatgttcatgactccccgccgccccgggcccacccgcaagcattaccagccctatgccccaccacgcgacttcgcagcctatcgctcc

SEQ ID NO: 8 - protein, 35 aa, synthetic construct
TKKKYSSSVHDPNGEYMFMRAVNTAKKSRLTDVTL

SEQ ID NO: 9 - DNA, 105 bp, synthetic construct
  source 1..105: mol_type = other DNA
acaaaaaagaagtattcatccagtgtgcacgaccctaacggtgaatacatgttcatgagagcagtgaacacagccaaaaaatccagactcacagatgtgacccta

SEQ ID NO: 10 - DNA, 105 bp, synthetic construct
  source 1..105: mol_type = other DNA
acaaaaaagaagtattcatccagtgtgcacgaccctaacggtgaatacatgttcatgagagcagtgaacacagccaaaaaatctagactcacagatgtgacccta

SEQ ID NO: 11 - protein, 35 aa, synthetic construct
TKKKYSSSVHDPNGEYMNMRAVNTAKKSRLTDVTL

SEQ ID NO: 12 - DNA, 105 bp, synthetic construct
  source 1..105: mol_type = other DNA
acaaaaaagaagtattcatccagtgtgcacgaccctaacggtgaatacatgaacatgagagcagtgaacacagccaaaaaatccagactcacagatgtgacccta

SEQ ID NO: 13 - protein, 117 aa, synthetic construct
TKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 14 - DNA, 351 bp, synthetic construct
  source 1..351: mol_type = other DNA
accaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaat

SEQ ID NO: 15 - protein, 48 aa, synthetic construct
QRRKYRSNKGESPVEPAEPCRYSCPREEEGSTIPIQEDYRKPEPACSP

SEQ ID NO: 16 - DNA, 144 bp, synthetic construct
  source 1..144: mol_type = other DNA
caacgaaggaaatatagatcaaacaaaggagaaagtcctgtggagcctgcagagccttgtcgttacagctgccccagggaggaggagggcagcaccatccccatccaggaggattaccgaaaaccggagcctgcctgctccccc

SEQ ID NO: 17 - protein, 42 aa, synthetic construct
ALYLLRRDQRLPPDAHKPPGGGSFRTPIQEEQADAHSTLAKI

SEQ ID NO: 18 - DNA, 126 bp, synthetic construct
  source 1..126: mol_type = other DNA
gccctgtacctgctccgcagggaccagaggctgccccccgatgcccacaagccccctgggggaggcagtttcaggacccccatccaagaggagcaggccgacgcccactccaccctggccaagatc

SEQ ID NO: 19 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 23 - protein, 24 aa, synthetic construct
IYIWAPLAGTCGVLLLSLVITLYC

SEQ ID NO: 24 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 25 - protein, 45 aa, synthetic construct
TTTPAPRPPTPAPTIASQPLSLRPEACRPAAGGAVHTRGLDFACD

SEQ ID NO: 26 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 27 - protein, 27 aa, synthetic construct
FWVLVVVGGVLACYSLLVTVAFIIFWV

SEQ ID NO: 28 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 29 - protein, 4 aa, synthetic construct
GGSG

SEQ ID NO: 30 - protein, 5 aa, synthetic construct
GGSGG

SEQ ID NO: 31 - protein, 5 aa, synthetic construct
GSGSG

SEQ ID NO: 32 - protein, 5 aa, synthetic construct
GSGGG

SEQ ID NO: 33 - protein, 5 aa, synthetic construct
GSSSG

SEQ ID NO: 34 - protein, 5 aa, synthetic construct
GGGSG

SEQ ID NO: 35 - protein, 5 aa, synthetic construct
DKTHT

SEQ ID NO: 36 - protein, 4 aa, synthetic construct
CPPC

SEQ ID NO: 37 - protein, 15 aa, synthetic construct
CPEPKSCDTPPPCPR

SEQ ID NO: 38 - protein, 12 aa, synthetic construct
ELKTPLGDTTHT

SEQ ID NO: 39 - protein, 10 aa, synthetic construct
KSCDKTHTCP

SEQ ID NO: 40 - protein, 7 aa, synthetic construct
KCCVDCP

SEQ ID NO: 41 - protein, 7 aa, synthetic construct
KYGPPCP

SEQ ID NO: 42 - protein, 15 aa, synthetic construct
EPKSCDKTHTCPPCP

SEQ ID NO: 43 - protein, 12 aa, synthetic construct
ERKCCVECPPCP

SEQ ID NO: 44 - protein, 17 aa, synthetic construct
ELKTPLGDTTHTCPRCP

SEQ ID NO: 45 - protein, 12 aa, synthetic construct
SPNMVPHAHHAQ

SEQ ID NO: 46 - protein, 15 aa, synthetic construct
EPKSCDKTYTCPPCP

SEQ ID NO: 47 - protein, 5 aa, synthetic construct
  REGION >5: note = The entire sequence of amino acids 1-5 can be repeated one or more times.
GSGGS

SEQ ID NO: 48 - protein, 4 aa, synthetic construct
  REGION >4: note = The entire sequence of amino acids 1-4 can be repeated one or more times.
GGGS

SEQ ID NO: 49 - protein, 5 aa, synthetic construct
  REGION >5: note = The entire sequence of amino acids 1-5 can be repeated one or more times.
GGGGS

SEQ ID NO: 50 - protein, 15 aa, synthetic construct
GGGGSGGGGSGGGGS

SEQ ID NO: 51 - DNA, 45 bp, synthetic construct
  source 1..45: mol_type = other DNA
ggtggcggtggctcgggcggtggtgggtcgggtggcggcggatct

SEQ ID NO: 52 - DNA, 21 bp, synthetic construct
  source 1..21: mol_type = other DNA
agatccaggatactgagggca

SEQ ID NO: 53 - DNA, 21 bp, synthetic construct
  source 1..21: mol_type = other DNA
tctctggcctggtactggcta

SEQ ID NO: 54 - DNA, 21 bp, synthetic construct
  source 1..21: mol_type = other DNA
gcttctgcatcacaagtcaga

SEQ ID NO: 55 - DNA, 20 bp, synthetic construct
  source 1..20: mol_type = other DNA
tatctcttgtactacactga

SEQ ID NO: 56 - DNA, 20 bp, synthetic construct
  source 1..20: mol_type = other DNA
gctcttggagccaaccgttg

SEQ ID NO: 57 - DNA, 21 bp, synthetic construct
  source 1..21: mol_type = other DNA
cttcctcaagggctgccagga

SEQ ID NO: 58 - DNA, 20 bp, synthetic construct
  source 1..20: mol_type = other DNA
cctacatgccccccacctcc

SEQ ID NO: 59 - DNA, 20 bp, synthetic construct
  source 1..20: mol_type = other DNA
cgctcgcatcctccacgaac

SEQ ID NO: 60 - DNA, 21 bp, synthetic construct
  source 1..21: mol_type = other DNA
gtgagcagcctcacaagacag

SEQ ID NO: 61 - DNA, 20 bp, synthetic construct
  source 1..20: mol_type = other DNA
ccttggggctctgacaggta

SEQ ID NO: 62 - DNA, 21 bp, synthetic construct
  source 1..21: mol_type = other DNA
ccagaacactcgggtgcctcg

SEQ ID NO: 63 - DNA, 21 bp, synthetic construct
  source 1..21: mol_type = other DNA
caaggccgtgcagaacaaagt

SEQ ID NO: 64 - DNA, 21 bp, synthetic construct
  source 1..21: mol_type = other DNA
ttctgcacggccttggaaatg

SEQ ID NO: 65 - DNA, 21 bp, synthetic construct
  source 1..21: mol_type = other DNA
cctgcacccctgagcctgtga

SEQ ID NO: 66 - DNA, 21 bp, synthetic construct
  source 1..21: mol_type = other DNA
gaggatctagaggacgaggag

SEQ ID NO: 67 - DNA, 21 bp, synthetic construct
  source 1..21: mol_type = other DNA
catcctggtgactctgctcct

SEQ ID NO: 68 - DNA, 21 bp, synthetic construct
  source 1..21: mol_type = other DNA
tccagccggccctgctgctgg

SEQ ID NO: 69 - DNA, 1707 bp, synthetic construct
  source 1..1707: mol_type = other DNA
atggccttaccagtgaccgccttgctcctgccgctggccttgctgctccacgccgccaggccgggatcccaggtgcagctgcagcagtctgatgccgagctcgtgaagcctggcagcagcgtgaagatcagctgcaaggccagcggctacaccttcaccgaccacgccatccactgggtcaagcagaagcctgagcagggcctggagtggatcggccacttcagccccggcaacaccgacatcaagtacaacgacaagttcaagggcaaggccaccctgaccgtggacagaagcagcagcaccgcctacatgcagctgaacagcctgaccagcgaggacagcgccgtgtacttctgcaagaccagcaccttctttttcgactactggggccagggcacaaccctgacagtgtctagcggaggcggaggatctggcggcggaggaagtggcggagggggatctgaactcgtgatgacccagagccccagctctctgacagtgacagccggcgagaaagtgaccatgatctgcaagtcctcccagagcctgctgaactccggcgaccagaagaactacctgacctggtatcagcagaaacccggccagccccccaagctgctgatcttttgggccagcacccgggaaagcggcgtgcccgatagattcacaggcagcggctccggcaccgactttaccctgaccatcagctccgtgcaggccgaggacctggccgtgtattactgccagaacgactacagctaccccctgaccttcggagccggcaccaagctggaactgaagtccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaccaaaaggaaaaaacagaggagtcggagaaatgatgaggagctggagacaagagcccacagagtagctactgaagaaaggggccggaagccccaccaaattccagcttcaacccctcagaatccagcaacttcccaacatcctcctccaccacctggtcatcgttcccaggcacctagtcatcgtcccccgcctcctggacaccgtgttcagcaccagcctcagaagaggcctcctgctccgtcgggcacacaagttcaccagcagaaaggcccgcccctccccagacctcgagttcagccaaaacctccccatggggcagcagaaaactcattgtccccttcctctaatatcgatagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 70 - protein, 569 aa, synthetic construct
MALPVTALLLPLALLLHAARPGSQVQLQQSDAELVKPGSSVKISCKASGYTFTDHAIHWVKQKPEQGLEWIGHFSPGNTDIKYNDKFKGKATLTVDRSSSTAYMQLNSLTSEDSAVYFCKTSTFFFDYWGQGTTLTVSSGGGGSGGGGSGGGGSELVMTOSPSSLTVTAGEKVTMICKSSQSLLNSGDQKNYLTWYOQKPGQPPKLLIFWASTRESGVPDRFTGSGSGTDFTLTISSVQAEDLAVYYCONDYSYPLTFGAGTKLELKSGTTTPAPRPPTPAPTIASQPLSLRPEACRPAAGGAVHTRGLDFACDIYIWAPLAGTCGVLLLSLVITLYCTKRKKQRSRRNDEELETRAHRVATEERGRKPHQIPASTPONPATSQHPPPPPGHRSQAPSHRPPPPGHRVQHQPQKRPPAPSGTQVHQQKGPPLPRPRVQPKPPHGAAENSLSPSSNIDRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 485 aa, synthetic construct
MALPVTALLLPLALLLHAARPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGGGGSGGGGSGGGGSGGGGSDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 485 aa, synthetic construct
MALPVTALLLPLALLLHAARPQVQLQQSGAEVKKPGASVKVSCKASGYTFTGYYMHWVRQAPGQGLEWMGWINPNSGGTNYAQNFQGRVTMTRDTSISTAYMELRRLRSDDTAVYYCASGWDFDYWGQGTLVTVSSGGGGSGGGGSGGGGSGGGGSDIRMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 73 - protein, 237 aa, synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSSGGGGSGGGGSSGGGSDIQMTQSPSTLSASVGDRVTITCKASQDVGTAVDWYQQKPGQAPKLLIYWASTRHTGVPDRFSGSGSGTDFTLTISRLQPEDFAVYYCQQYNSYPLTFGQGTKVDIK

SEQ ID NO: 74 - DNA, 711 bp, synthetic construct
  source 1..711: mol_type = other DNA
gaggtccagctggtgcagtctggagctgaggtgaagaagcctggggcctcagtgaaggtctcctgcaaggcttctggatacacattcactgaatacaccatccactgggtgaggcaggcccctggaaagggccttgagtggattggaaacattaatcctaacaatggtggtactacctacaaccagaagttcgaggacagagtcacaatcactgtagacaagtccaccagcacagcctacatggagctcagcagcctgagatctgaggatactgcagtctattactgtgcagctggttggaactttgactactggggccaaggcaccacggtcaccgtctcctcaggaggcggaggatctggcggcggaggaagttctggcggaggcagcgacattcagatgacccagtctcccagcaccctgtccgcatcagtaggagacagggtcaccatcacttgcaaggccagtcaggatgtgggtactgctgtagactggtatcaacagaaaccagggcaagctcctaaactactgatttactgggcatccacccggcacactggagtccctgatcgcttcagcggcagtggatctgggacagatttcactctcaccatcagcagactgcagcctgaagactttgcagtttattactgtcagcaatataacagctatcctctcacgttcggccaggggaccaaggtggatatcaaa

SEQ ID NO: 75 - protein, 241 aa, synthetic construct
QVQLQQSGAEVKKPGASVKVSCKASGYTFTGYYMHWVRQAPGQGLEWMGWINPNSGGTNYAQNFQGRVTMTRDTSISTAYMELRRLRSDDTAVYYCASGWDFDYWGQGTLVTVSSGGGGSGGGGSGGGGSGGGGSDIRMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 76 - protein, 201 aa, synthetic construct
MGRGLLRGLWPLHIVLWTRIASTIPPHVOKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSSG

SEQ ID NO: 77 - DNA, 603 bp, synthetic construct
  source 1..603: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcatccgga

SEQ ID NO: 78 - protein, 238 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVFWVLVVVGGVLACYSLLVTVAFIIFWVRSKRSRLLHSDYMNMTPRRPGPTRKHYQPYAPPRDFAAYRS

SEQ ID NO: 79 - DNA, 714 bp, synthetic construct
  source 1..714: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtgttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 80 - protein, 232 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTVLCGAISLAPKAQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAVRS

SEQ ID NO: 81 - DNA, 696 bp, synthetic construct
  source 1..696: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatcaggagtaagaggagcaggctcctgcacagtgactacatgaacatgactccccgccgccccgggcccacccgcaagcattaccagccctatgccccaccacgcgacttcgcagcctatcgctcc

SEQ ID NO: 82 - protein, 232 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTVLCGAISLAPKLQIKESLRAELRVTERRAEVPTAHPSPSPRPAGQFQTLVVGVVGGLLGSLVLLVWVLAVIRSKRSRLLHSDYMNMTPRRPGPTRKHYQPYAPPRDFAAVRS

SEQ ID NO: 83 - DNA, 693 bp, synthetic construct
  source 1..693: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatcaggagtaagaggagcaggctcctgcacagtgactacatgaacatgactccccgccgccccgggcccacccgcaagcattaccagccctatgccccaccacgcgacttcgcagcctatcgc

SEQ ID NO: 84 - protein, 237 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVIYIWAPLAGTCGVLLLSLVITLYCKKRGRKKLLYIFKQPFMRPVQTTQEEDGCSCRFPEEEEGGCEL

SEQ ID NO: 85 - DNA, 711 bp, synthetic construct
  source 1..711: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggttatctacatctgggcgcccttggccgggacttgtggggtccttctcctgtcactggttatcaccctttactgcaaaaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 86 - protein, 237 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTVLCGAISLAPKLQIKESLRAELRVTERRAEVPTAHPSPSPRPAGQFQTLVIYIWAPLAGTCGVLLLSLVITLVCKKRGRKKLLYIFKQPFMRPVQTTQEEDGCSCRFPEEEEGGCEL

SEQ ID NO: 87 - DNA, 711 bp, synthetic construct
  source 1..711: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaagctgcagatcaaagagagcctgcgggcagagctcagggtgacagagagaagggcagaagtgcccacagcccaccccagcccctcacccaggccagccggccagttccaaaccctggttatctacatctgggcgcccttggccgggacttgtggggtccttctcctgtcactggttatcaccctttactgcaaaaaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 88 - protein, 238 aa, synthetic construct
MEAAVAAPRPRLLLLVLAAAAAAAAALLPGATALQCFCHLCTKDNFTCVTDGLCFVSVTETTDKVIHNSMCIAEIDLIPRDRPFVCAPSSKTGSVTTTYCCNQDHCNKIELPTTVKSSPGLGPVELAAVIAGPVCFVCISLMLMVYIRAARHLCPPLPTPCASSAIEFPGGKETWOWINPVDFQEEASLQEALVVEMSWDKGERTEPLEKTELPEGAPELALDTELSLEDGDRCKAKM

SEQ ID NO: 89 - DNA, 714 bp, synthetic construct
  source 1..714: mol_type = other DNA
atggaggcggcggtcgctgctccgcgtccccggctgctcctcctcgtgctggcggcggcggcggcggcggcggcggcgctgctcccgggggcgacggcgttacagtgtttctgccacctctgtacaaaagacaattttacttgtgtgacagatgggctctgctttgtctctgtcacagagaccacagacaaagttatacacaacagcatgtgtatagctgaaattgacttaattcctcgagataggccgtttgtatgtgcaccctcttcaaaaactgggtctgtgactacaacatattgctgcaatcaggaccattgcaataaaatagaacttccaactactgtaaagtcatcacctggccttggtcctgtggaactggcagctgtcattgctggaccagtgtgcttcgtctgcatctcactcatgttgatggtctatatcagggccgcacggcacctgtgcccgccgctgcccacaccctgtgccagctccgccattgagttccctggagggaaggagacttggcagtggatcaacccagtggacttccaggaagaggcatccctgcaggaggccctggtggtagagatgtcctgggacaaaggcgagaggactgagcctctcgagaagacagagctacctgagggtgcccctgagctggccctggatacagagttgtccttggaggatggagacaggtgcaaggccaagatg

SEQ ID NO: 90 - protein, 403 aa, synthetic construct
MGRGLLRGLWPLHIVLWTRIASTIPPHVOKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYQQKVFVLLAALRPOWCSREIPDPANSTCAKKYPIAEEKTOLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 91 - DNA, 1209 bp, synthetic construct
  source 1..1209: mol_type = other DNA
atgggtcgggggctgctcaggggcctgtggccgctgcacatcgtcctgtggacgcgtatcgccagcacgatcccaccgcacgttcagaagtcggttaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctaccagcaaaaggtgtttgttctcctagcagccctcagacctcagtggtgtagcagagaaattccagatccagcaaatagcacttgcgctaagaaatatcccattgcagaggagaagacacagctgcccttggacaggctcctgatagactggcccacgcctgaagatcctgaaccgctggtcatcagtgaagtccttcatcaagtgaccccagttttcagacatcccccctgctccaactggccacaaagggaaaaaggaatccaaggtcatcaggcctctgagaaagacatgatgcacagtgcctcaagcccaccacctccaagagctctccaagctgagagcagacaactggtggatctgtacaaggtgctggagagcaggggctccgacccaaagccagaaaacccagcctgtccctggacggtgctcccagcaggtgaccttcccacccatgatggctacttaccctccaacatagatgacctcccctcacatgaggcacctctcgctgactctctggaagaactggagcctcagcacatctccctttctgttttcccctcaagttctcttcacccactcaccttctcctgtggtgataagctgactctggatcagttaaagatgaggtgtgactccctcatgctc

(19) United States
(12) Patent Application Publication
(10) Pub. No.: US 2023/0190809 A1
(43) Pub. Date: Jun. 22, 2023

(54) ALTERNATIVE GENERATION OF ALLOGENEIC HUMAN T CELLS

(71) Applicant: Tmunity Therapeutics Inc., Philadelphia, PA (US)

(72) Inventors: Felipe Bedoya, Eagleville, PA (US); David Barrett, Philadelphia, PA (US); Vijay Peddareddigari, Philadelphia, PA (US)

(73) Assignee: Tmunity Therapeutics Inc., Philadelphia, PA (US)

(21) Appl. No.: 17/940,186

(22) Filed: Sep. 8, 2022

Related U.S. Application Data
(60) Provisional application No. 63/242,909, filed on Sep. 10, 2021.

(51) Int. Cl. A61K 35/17; C07K 14/725; C07K 14/705; C07K 14/73; C12N 15/11; C12N 9/22

(52) U.S. Cl. CPC A61K 35/17; C07K 14/7051; C07K 14/70517; C07K 14/70514; C12N 15/11; C12N 9/22; C07K 2317/622; C07K 2319/03; C12N 2310/20

(57) ABSTRACT

The present invention provides gene edited modified immune cells suitable for adoptive T cell therapy comprising a nucleic acid capable of downregulating CD3δ, CD3ε, CD3γ, B2M, CIITA, TAP1, TAP2, TAPBP, NLRC5, HLA-DM, RFX5, RFXANK, RFXAP, and invariant chain; and further comprising an exogenous nucleic acid encoding a chimeric antigen receptor (CAR), an engineered T cell receptor (TCR), a Killer cell immunoglobulin-like receptor (KIR), dominant negative receptor and/or a switch receptor. Also provided are compositions and methods for generating the modified immune cell, and methods of using the modified immune cells for adoptive therapy and treating a disease or condition.

### CROSS-REFERENCE TO RELATED APPLICATION

The present application claims the benefit of priority of U.S. Provisional Application No. 63/242,909, filed on Sep. 10, 2021, the contents of which are specifically incorporated by reference.

### SEQUENCE LISTING

The specification further incorporates by reference the Sequence Listing submitted herewith. The Sequence Listing .xml file identified as 125400-1426, is 96,777 bytes in size and was created on Dec. 14, 2022. The Sequence Listing, electronically filed herewith does not extend beyond the scope of the specification, and does not contain new matter.

### BACKGROUND

Adoptive immunotherapy involves the transfer of autologous antigen-specific T-cells generated ex vivo back into a patient, and has been shown to be a promising strategy for the treatment of cancers, infections and auto-immune diseases. T-cells used for adoptive immunotherapy are primary cells engineered to express a Chimeric Antigen Receptor (CAR), or a recombinant T cell Receptor (TCR) and expand ex vivo to redirect primary immune cells against pathological cells, such as cancer cells. CARs are synthetic antibody-like molecules consisting of a targeting moiety that is associated with one or more signaling domains in a single fusion molecule, and are designed to convey antigen specificity to T cells. CARs have successfully allowed T cells to be redirected against antigens expressed at the surface of tumor cells from various malignancies, including lymphomas and solid tumors. T cells expressing CARs also exhibit long-term efficacy for the treatment of certain types of cancers.

However, adoptive immunotherapy is currently based on autologous cell transfer. In autologous immunotherapy, a patient receives a personalized treatment based on the patient's own lymphocytes, which were isolated from the patient, genetically modified or selected ex vivo, cultivated in vitro and infused back into the patient. Despite approval and general success of autologous adoptive immunotherapy therapies, the scalability and feasibility of such therapies present significant challenges. Autologous adoptive immunotherapy therapy is still fairly complicated—requiring expertise and clinical management, and expensive dedicated facilities. Many patients also are unable to receive adoptive immunotherapy due to rapid disease progression during CAR manufacturing, or the degradation of the patient's immune function prior to such treatment. As such, the widespread clinical application of cancer immunotherapy is limited by the considerable economic constraint imposed by the personalized preparation of autologous CART-cells. Accordingly, there is a need for a standardized adoptive immunotherapy in which allogeneic therapeutic cells are pre-manufactured, characterized in detail, and available for immediate administration to a broad range of patients.

Allogeneic immunotherapy remains a dangerous procedure with many possible complications, such as allogeneic T-cell responses, which is clinically manifested as graft-versus-host disease (GVHD) and/or Host-versus-Graft disease (HvGD, graft rejection). GvHD is caused by the attack of recipient tissues by infused allogeneic CAR-T cell mediated by alloreactive TCR on donor CAR cells. In particular, endogenous T-cell receptor alpha (TCRα; TRAC) and beta (TCRβ; TRBC) chains on infused T cells may recognize major and minor histocompatibility antigens in the recipient, leading to (GvHD). Conversely, infused allogeneic CART cells may be rejected by the recipient T lymphocytes leading to HvGD. As such, the applicability and versatility of adoptive immunotherapy will be improved by the use of allogeneic CAR T cells if and when a standardized solution that controls inherent allogeneic immune responses is identified. The specific inhibition of GvHD will enable the safe and effective use of allogeneic CART-cells.

Accordingly, a need exists for improved methods of CAR-T cells that does not invoke a host immune response. Specifically, there is a need for novel and alternative compositions and methods for generating allogeneic T cells with improved fitness.

The present invention provides methods and compositions that address these needs.

### SUMMARY

In one aspect, the present disclosure provides a modified immune cell comprising: (a) an insertion and/or deletion in one or more gene loci, each encoding an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain). The insertion and/or deletion is capable of downregulating gene expression of the one or more endogenous immune genes. In addition, the modified immune cell comprises (b) an exogenous nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen. In some embodiments, the modified immune cell further comprises a dominant negative receptor, a switch receptor, a chemokine, a chemokine receptor, a cytokine, a cytokine receptor, IL-7, IL-7R, IL-15, IL-15R, IL-21, IL-18, CCL21, CCL19, or any combination thereof.

In some embodiments, the insertion and/or deletion is capable of downregulating the gene expression of: (a) a T cell receptor subunit selected from CD3δ, CD3ε, and/or CD3γ; (b) a HLA class I molecule selected from B2M, TAP1, TAP2, TAPBP, and/or NLRC5; and (c) a HLA class II molecule selected from HLA-DM, RFX5, RFXANK, RFXAP, and/or invariant chain (Ii Chain).

In some embodiments, the insertion and/or deletion is capable of downregulating (a) the gene expression of CD3δ, and (b) the gene expression of a HLA molecule selected from the group consisting of B2M, TAP1, TAP2, TAPBP, NLRC5, HLA-DM, RFX5, RFXANK, RFXAP, invariant chain (Ii Chain), and any combination thereof.

In some embodiments, the insertion and/or deletion is capable of downregulating: (a) the gene expression of CD3ε, and (b) the gene expression of a HLA molecule selected from the group consisting of B2M, TAP1, TAP2, TAPBP, NLRC5, CIITA, HLA-DM, RFX5, RFXANK, RFXAP, invariant chain (Ii Chain), or any combination thereof.

In some embodiments, the insertion and/or deletion is capable of downregulating: (a) the gene expression of CD3γ, and (b) the gene expression of a HLA molecule selected from the group consisting of B2M, TAP1, TAP2, TAPBP, NLRC5, CIITA, HLA-DM, RFX5, RFXANK, RFXAP, invariant chain (Ii Chain), or any combination thereof.

In some embodiments, the insertion and/or deletion is capable of downregulating the gene expression of: (a) CD3ε, B2M, and CIITA; (b) CD3ε, B2M, and RFX5; (c) CD3ε, B2M, and RFXAP; (d) CD3ε, B2M, and RFXANK; (e) CD3ε, B2M, and HLA-DM; (f) CD3ε, B2M, and Ii chain; (g) CD3ε, TAP1, and CIITA; (h) CD3ε, TAP1, and RFX5; (i) CD3ε, TAP1, and RFXAP; (j) CD3ε, TAP1, and RFXANK; (k) CD3ε, TAP1, and HLA-DM; (1) CD3ε, TAP1, and Ii chain; (m) CD3ε, TAP2, and CIITA; (n) CD3ε, TAP2, and RFX5; (o) CD3ε, TAP2, and RFXAP; (p) CD3ε, TAP2, and RFXANK; (q) CD3ε, TAP2, and HLA-DM; (r) CD3ε, TAP2, and Ii chain; (s) CD3ε, NLRC5, and CIITA; (t) CD3ε, NLRC5, and RFX5; (u) CD3ε, NLRC5, and RFXAP; (v) CD3ε, NLRC5, and RFXANK; (w) CD3ε, NLRC5, and HLA-DM; (x) CD3ε, NLRC5, and Ii chain; (y) CD3ε, TAPBP, and CIITA; (z) CD3ε, TAPBP, and RFX5; (aa) CD3ε, TAPBP, and RFXAP; (bb) CD3ε, TAPBP, and RFXANK; (cc) CD3ε, TAPBP, and HLA-DM; or (dd) CD3ε, TAPBP, and Ii chain.

In some embodiments, the insertion and/or deletion is capable of downregulating the gene expression of: (a) CD3δ, B2M, and CIITA; (b) CD3δ, B2M, and RFX5; (c) CD3δ, B2M, and RFXAP; (d) CD3δ, B2M, and RFXANK; (e) CD3δ, B2M, and HLA-DM; (f) CD3δ, B2M, and Ii chain; (g) CD3δ, TAP1, and CIITA; (h) CD3δ, TAP1, and RFX5; (i) CD3δ, TAP1, and RFXAP; (j) CD3δ, TAP1, and RFXANK; (k) CD3δ, TAP1, and HLA-DM; (1) CD3δ, TAP1, and Ii chain; (m) CD3δ, TAP2, and CIITA; (n) CD3δ, TAP2, and RFX5; (o) CD3δ, TAP2, and RFXAP; CD3δ, TAP2, and RFXANK; CD3δ, TAP2, and HLA-DM; (r) CD3δ, TAP2, and Ii chain; (s) CD3δ, NLRC5, and CIITA; (t) CD3δ, NLRC5, and RFX5; (u) CD3δ, NLRC5, and RFXAP; (v) CD3δ, NLRC5, and RFXANK; (w) CD3δ, NLRC5, and HLA-DM; (x) CD3δ, NLRC5, and Ii chain; (y) CD3δ, TAPBP, and CIITA; (z) CD3δ, TAPBP, and RFX5; (aa) CD3δ, TAPBP, and RFXAP; (bb) CD3δ, TAPBP, and RFXANK; (cc) CD3δ, TAPBP, and HLA-DM; or (dd) CD3δ, TAPBP, and Ii chain.

In some embodiments, the insertion and/or deletion is capable of downregulating the gene expression of: (a) CD3γ, B2M, and CIITA; (b) CD3γ, B2M, and RFX5; (c) CD3γ, B2M, and RFXAP; (d) CD3γ, B2M, and RFXANK; (e) CD3γ, B2M, and HLA-DM; (f) CD3γ, B2M, and Ii chain; (g) CD3γ, TAP1, and CIITA; (h) CD3γ, TAP1, and RFX5; (i) CD3γ, TAP1, and RFXAP; (j) CD3γ, TAP1, and RFXANK; (k) CD3γ, TAP1, and HLA-DM; (1) CD3γ, TAP1, and Ii chain; (m) CD3γ, TAP2, and CIITA; (n) CD3γ, TAP2, and RFX5; (o) CD3γ, TAP2, and RFXAP; (p) CD3γ, TAP2, and RFXANK; (q) CD3γ, TAP2, and HLA-DM; (r) CD3γ, TAP2, and Ii chain; (s) CD3γ, NLRC5, and CIITA; (t) CD3γ, NLRC5, and RFX5; (u) CD3γ, NLRC5, and RFXAP; (v) CD3γ, NLRC5, and RFXANK; (w) CD3γ, NLRC5, and HLA-DM; (x) CD3γ, NLRC5, and Ii chain; (y) CD3γ, TAPBP, and CIITA; (z) CD3γ, TAPBP, and RFX5; (aa) CD3γ, TAPBP, and RFXAP; (bb) CD3γ, TAPBP, and RFXANK; (cc) CD3γ, TAPBP, and HLA-DM; or (dd) CD3γ, TAPBP, and Ii chain.

In some embodiments, the modified immune cell is selected from the group consisting of a T cell, a natural killer cell (NK cell), a natural killer T cell, a lymphoid progenitor cell, a hematopoietic stem cell, a stem cell, a macrophage, a dendritic cell, or any combination thereof. In some embodiments, the modified immune cell is a CD4+ T cell or a CD8+ T cell. In some embodiments, the modified immune cell is an allogeneic T cell or autologous human T cell.

In some embodiments, the insertion and/or deletion is the result of gene editing selected from the group consisting of: (a) a CRISPR-associated (Cas) (CRISPR-Cas) endonuclease system and a guide RNA; (b) a TALEN gene editing system, a zinc finger nuclease (ZFN) gene editing system, a meganuclease gene editing system, or a mega-TALEN gene editing system; and (c) a gene silencing system selected from antisense RNA, antigomer RNA, RNAi, siRNA, or shRNA. In some embodiments, the CRISPR-Cas system comprises a pAd5/F35-CRISPR vector. In some embodiments, the Cas endonuclease comprises Cas3, Cas4, Cas8a, Cas8b, Cas9, Cas10, Cas10d, Cas12a, Cas12b, Cas12d, Cas12e, Cas12f, Cas12g, Cas12h, Cas12i, Cas13, Cas14, CasX, Cse1, Csy1, Csn2, Cpf1, C2c1, Csm2, Cmr5, Fok1, S. pyogenes Cas9, Staphylococcus aureus Cas9, MAD7 nuclease (a type V CRISPR nuclease), or any combination thereof.

In some embodiments, the CRISPR-Cas endonuclease system comprises a guide RNA. In some embodiments, the guide RNA comprises a guide sequence that is complementary with a sequence within the one or more gene loci each encoding the immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain). In some embodiments, the guide RNA is complementary with a sequence within one or more exons of CD3δ, CD3ε, or CD3γ. In some embodiments, the guide RNA is complementary with a sequence within exon 1 of CD3δ, CD3ε, or CD3γ.

In some embodiments, the complementary sequence of the guide RNA is within the CD3δ gene locus and the guide RNA comprises a nucleic acid sequence encoded by SEQ ID NO: 53. In some embodiments, the complementary sequence of the guide RNA is within the CD3ε gene locus and the guide RNA comprises a nucleic acid sequence encoded by SEQ ID NO: 52. In some embodiments, the complementary sequence of the guide RNA is within the CD3γ gene locus and the guide RNA comprises a nucleic acid sequence encoded by SEQ ID NO: 54. In some embodiments, the complementary sequence of the guide RNA is within the B2M gene locus and the guide RNA comprises a nucleic acid sequence encoded by SEQ ID NO: 55. In some embodiments, the complementary sequence of the guide RNA is within the CIITA gene locus and the guide RNA comprises a nucleic acid sequence encoded by SEQ ID NO: 61. In some embodiments, the complementary sequence of the guide RNA is within the TAP1 gene locus and the guide RNA comprises a nucleic acid sequence encoded by SEQ ID NO: 56. In some embodiments, the complementary sequence of the guide RNA is within the TAP2 gene locus and the guide RNA comprises a nucleic acid sequence encoded by SEQ ID NO: 57. In some embodiments, the complementary sequence of the guide RNA is within the TAPBP gene locus and the guide RNA comprises a nucleic acid sequence encoded by SEQ ID NO: 58, SEQ ID NO: 59, or a combination thereof. In some embodiments, the complementary sequence of the guide RNA is within the NLRCS gene locus and the guide RNA comprises a nucleic acid sequence encoded by SEQ ID NO: 60. In some embodiments, the complementary sequence of the guide RNA is within the HLA-DM gene locus and the guide RNA comprises a nucleic acid sequence encoded by SEQ ID NO: 62. In some embodiments, the complementary sequence of the guide RNA is within the RFX5 gene locus and the guide RNA comprises a nucleic acid sequence encoded by SEQ ID NO: 63, SEQ ID NO: 64, or a combination thereof. In some embodiments, the complementary sequence of the guide RNA is within the RFXANK gene locus and the guide RNA comprises a nucleic acid sequence encoded by SEQ ID NO: 65. In some embodiments, the complementary sequence of the guide RNA is within the RFXAP gene locus and the guide RNA comprises a nucleic acid sequence encoded by SEQ ID NO: 66. In some embodiments, the complementary sequence of the guide RNA is within the Ii Chain gene locus and the guide RNA comprises a nucleic acid sequence encoded by SEQ ID NO: 67, SEQ ID NO: 68, or any combination thereof.

In one aspect of the present disclosure, the modified immune cell exerts a reduced immune response in a subject when the modified immune cell is administered to the subject, as compared to the immune response exerted by an unmodified immune cell administered to the same subject.

In some embodiments, the modified immune cell exerts a reduced immune response in a subject when the modified immune cell is administered to the subject, as compared to the immune response exerted by an immune cell comprising an insertion and/or deletion capable of downregulating the gene expression of TRAC, TRBC, B2M, and CIITA. In some embodiments, the immune response is a graft-versus-host disease (GvHD) response. In some embodiments, the reduced GvHD response by the modified immune cell is compared to an equivalent immune cell without the deletion and/or insertion in one or more gene loci, or an immune cell comprising the deletion and/or insertion in TRAC, TRBC, B2M, and CIITA. In some embodiments, the reduced GvHD response is elicited against an HLA-I mismatched cell or against an HLA-II mismatched cell.

In some embodiments, the GvHD response is reduced by about 10% or more, about 20% or more, about 30% or more, about 40% or more, about 50% or more, about 60% or more, about 70% or more, about 80% or more, about 90% or more, or about 95% or more. In some embodiments, the GvHD response is reduced by about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 20-fold or more, about 30-fold or more, about 50-fold or more, about 100-fold or more, about 150-fold or more, or about 200-fold or more.

In one aspect of the present disclosure, the exogenous nucleic acid encodes a chimeric antigen receptor (CAR). In some embodiments, the CAR comprises an antigen binding domain, a hinge domain, a transmembrane domain, a costimulatory signaling domain, and an intracellular signaling domain. In some embodiments, the antigen-binding domain comprises a full length antibody or an antigen-binding fragment thereof, a Fab, a F(ab)₂, a monospecific Fab₂, a bispecific Fab₂, a trispecific Fab₂, a single-chain variable fragment (scFv), a diabody, a triabody, a minibody, a V-NAR, or a VhH.

In some embodiments of the modified immune cell, the transmembrane domain is selected from an artificial hydrophobic sequence, a transmembrane domain of a type I transmembrane protein, an alpha, beta, or zeta chain of a T cell receptor, CD28, CD3 epsilon, CD45, CD4, CD2, CD5, CD8, CD9, CD16, CD22, CD33, CD37, CD64, CD80, CD86, OX40 (CD134), 4-1BB (CD137), ICOS (CD278), CD154, CD357 (GITR), Toll-like receptor 1 (TLR1), TLR2, TLR3, TLR4, TLR5, TLR6, TLR7, TLR8, TLR9, and a transmembrane domain derived from a killer immunoglobulin-like receptor (KIR).

In some embodiments of the modified immune cell, the costimulatory domain comprises one or more of a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof.

In some embodiments of the modified immune cell, the intracellular signaling domain comprises an intracellular domain selected from the group consisting of cytoplasmic signaling domains of a human CD2, CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof. In some embodiments, the intracellular signaling domain comprises a human CD3 zeta chain (CD3ζ).

In some embodiments of the modified immune cell, the antigen binding domain targets a tumor antigen associated with a hematologic malignancy, and/or associated with a solid tumor. In some embodiments, the antigen binding domain targets a tumor antigen selected from the group consisting of ROR1, mesothelin, c-Met, PSMA, PSCA, Folate receptor alpha, Folate receptor beta, EGFR, EGFRvIII, GPC2, GPC2, Mucin 1 (MUC1), Tn antigen ((Tn Ag) or (GalNAca-Ser/Thr)), TnMUC1, GDNF family receptor alpha-4 (GFRa4), fibroblast activation protein (FAP), and Interleukin-13 receptor subunit alpha-2 (IL-13Ra2 or CD213A2).

In some embodiments, the CAR comprises (a) a PSMA antigen binding domain, a CD2 costimulatory domain, and a CD3 zeta intracellular signaling domain; or (b) a mesothelin antigen binding domain, a 4-1BB costimulatory domain, and a CD3 zeta signaling domain; or (c) a TnMUC1 antigen binding domain, a CD2 costimulatory domain, and a CD3 zeta signaling domain.

In one aspect of the present disclosure, the modified immune cell further comprises a switch receptor. In some embodiments, the switch receptor comprises an extracellular domain of a signaling protein associated with a negative signal, a transmembrane domain, and an intracellular domain of a signaling protein associated with a positive signal.

In some embodiments, the modified immune cell further comprises a dominant negative receptor. In some embodiments, the dominant negative receptor comprises (a) a truncated variant of a wild-type protein associated with a negative signal; or (b) a variant of a wild-type protein associated with a negative signal comprising an extracellular domain, a transmembrane domain, and substantially lacking an intracellular signaling domain; or (c) an extracellular domain of a signaling protein associated with a negative signal, and a transmembrane domain. In some embodiments, the dominant negative receptor is PD1, VSIG3, VISG8, or TGFβR dominant negative receptor.

In some embodiments, the protein associated with the negative signal is selected from the group consisting of CTLA4, PD-1, TGFγRII, BTLA, VSIG3, VSIG8, and TIM-3. In some embodiments, the protein associated with the positive signal is selected from the group consisting of CD28, 4-1BB, IL12Rβ1, IL12Rβ2, CD2, ICOS, and CD27.

In some embodiments, the switch receptor is selected from the group consisting of PD-1-CD28, PD-1^{A132L}-CD28, PD-1-CD27, PD-1^{A132L}-CD27, PD-1-4-1BB, PD-1^{A132L}-4-1BB, PD-1-ICOS, PD-1^{A132L}-ICOS, PD-1-IL12Rβ1, PD-1^{A132L}-IL12Rβ1, PD-1-IL12Rβ2, PD-1^{A132L}-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2.

In some embodiments of the modified immune cell, the transmembrane domain of the switch receptor is selected from a transmembrane domain of a protein selected from the group consisting of CTLA4, PD-1, VSIG3, VSIG8, TGFβRII, BTLA, TIM-3, CD28, 4-1BB, IL12Rβ1, IL12Rβ2, CD2, ICOS, and CD27. In some embodiments, the transmembrane domain of the switch receptor is selected from the transmembrane of the protein associated with a negative signal or the transmembrane domain of the protein associated with the negative signal.

In one aspect, the present disclosure provides an isolated modified T cell, comprising at least one functionally impaired polypeptide selected from the group consisting of CD3δ, CD3ε, CD3γ, B2M, C2TA, TAP1, TAP2, TAPBP, NLRC5, HLA-DM, RFX5, RFXANK, RFXAP, and invariant chain (Ii Chain). In some embodiments, the modified T cell comprising the functionally impaired polypeptide exhibits at least one of (i) reduced T cell receptor expression as compared to an unmodified T cell; (ii) reduced expression of the impaired polypeptide; (iii) complete absence of the T cell receptor complex surface expression; and/or (iv) reduced or insufficient T cell receptor cross-linking. In some embodiments, the T cell exerts a reduced immune response in a subject when the modified T cell is administered to the subject, as compared to the immune response exerted by an unmodified T cell administered to the same subject.

In some embodiments, the T cell comprises two or more functionally impaired polypeptides, and wherein the second impaired polypeptide is T-cell receptor α chain (TRAC) and/or T-cell receptor β chain (TRBC).

In some embodiments, the modified T cell comprises: (a) three or more functionally impaired polypeptides selected from TRAC, CD3δ, CD3ε, CD3γ, B2M, C2TA, TAP1, TAP2, TAPBP, NLRC5, HLA-DM, RFX5, RFXANK, RFXAP, or Ii chain; or (b) two functionally impaired polypeptides selected from CD3α, CD3δ, CD3ε, CD3γ, B2M, C2TA, TAP1, TAP2, TAPBP, NLRC5, HLA-DM, RFX5, RFXANK, RFXAP, or Ii chain; or (c) three functionally impaired polypeptides selected from CD3α, CD3δ, CD3ε, CD3γ, B2M, C2TA, TAP1, TAP2, TAPBP, NLRC5, HLA-DM, RFX5, RFXANK, RFXAP, or Ii chain; or (d) a functionally impaired polypeptide selected from the group consisting of CD3δ, CD3ε, and CD3γ, and at least one functionally impaired polypeptide selected from TRAC, TRBC, B2M, C2TA, TAP1, TAP2, TAPBP, NLRC5, HLA-DM, RFX5, RFXANK, RFXAP, or Ii Chain; or (e) a functionally impaired polypeptide selected from the group consisting of CD3δ, CD3ε, and CD3γ, and a functionally impaired polypeptide selected from TRAC, TRBC, B2M, C2TA, TAP1, TAP2, TAPBP, NLRC5, HLA-DM, RFX5, RFXANK, RFXAP, or Ii Chain.

In some embodiments, the modified T cell comprises: (a) functionally impaired CD3δ and at least one functionally impaired polypeptide selected from TRAC, TRBC, B2M, C2TA, TAP1, TAP2, TAPBP, NLRC5, HLA-DM, RFX5, RFXANK, RFXAP, or Ii chain; (b) functionally impaired CD3ε and at least one functionally impaired polypeptide selected from TRAC, TRBC, B2M, C2TA, TAP1, TAP2, TAPBP, NLRC5, HLA-DM, RFX5, RFXANK, RFXAP, or Ii chain; or (c) functionally impaired CD3γ and at least one functionally impaired polypeptide selected from TRAC, B2M, C2TA, TAP1, TAP2, TAPBP, NLRC5, HLA-DM, RFX5, RFXANK, RFXAP, or Ii chain.

In some embodiments, the modified T cell comprises two or more functionally impaired polypeptides selected from TRAC, TRBC, B2M, C2TA, TAP1, TAP2, TAPBP, NLRC5, HLA-DM, RFX5, RFXANK, RFXAP, or Ii chain. In some embodiments, the modified T cell has a reduced expression of TRAC, TRBC, CD3δ, CD3ε, CD3γ, B2M, C2TA, TAP1, TAP2, TAPBP, NLRC5, HLA-DM, RFX5, RFXANK, RFXAP, Ii chain, or any combination thereof. In some embodiments, the modified T cell does not express CD3δ, CD3ε, CD3γ, TRAC, B2M, C2TA, TAP1, TAP2, TAPBP, NLRC5, HLA-DM, RFX5, RFXANK, RFXAP, Ii chain, or any combination thereof.

In some embodiments, the modified T cell further comprises a functionally impaired polypeptide selected from TRAC, TRBC, B2M, and C2TA. In some embodiments, the modified T cell has a reduced expression of TRAC, TRBC, B2M, or C2TA or does not express TRAC, TRBC, B2M, or C2TA. In some embodiments, the modification of CD3δ, CD3ε, and/or CD3γ leads to an impaired TCR/CD3 complex function. In some embodiments, at least one of CD3δ, CD3ε, or CD3γ is modified by targeting one or more exons of CD3δ, CD3ε, or CD3γ, optionally exon 1 of CD3δ, CD3ε, or CD3γ.

One aspect of the present disclosure provides a method for generating a modified immune cell comprising: (a) introducing into the immune cell one or more nucleic acids capable of downregulating gene expression of one or more endogenous immune genes encoding an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain); (b) introducing into the immune cell an exogenous nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen; and (c) expanding the modified immune cell to generate a population of T cells. In some embodiments, the method for generating a modified immune cell further comprising introducing into the immune cell an exogenous nucleic acid encoding a dominant negative receptor, a switch receptor, or a combination thereof.

In some embodiments, the one or more nucleic acids introduced into the modified immune cell are capable of downregulating the gene expression of: (a) a T cell receptor subunit selected from CD3δ, CD3ε, or CD3γ; and/or (b) a HLA class I molecule selected from B2M, TAP1, TAP2, TAPBP, or NLRC5; and/or (c) a HLA class II molecule selected from HLA-DM, RFX5, RFXANK, RFXAP, or invariant chain (Ii Chain).

In some embodiments, the one or more nucleic acids introduced into the modified immune cell are capable of downregulating the gene expression of CD3δ, and the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof. In some embodiments, the one or more nucleic acids introduced into the modified immune cell are capable of downregulating the gene expression of CD3ε, and a HLA molecule selected from the group consisting of B2M, TAP1, TAP2, TAPBP, NLRC5, CIITA, HLA-DM, RFX5, RFXANK, RFXAP, invariant chain (Ii Chain), and a combination thereof. In some embodiments, the one or more nucleic acids introduced into the modified immune cell are capable of downregulating the gene expression of CD3γ, and a HLA molecule selected from the group consisting of B2M, TAP1, TAP2, TAPBP, NLRC5, CIITA, HLA-DM, RFX5, RFXANK, RFXAP, invariant chain (Ii Chain), and a combination thereof.

In some embodiments, the one or more nucleic acids introduced into the modified immune cell are capable of downregulating the gene expression of (a) CD3ε, B2M, and CIITA; (b) CD3ε, B2M, and RFX5; (c) CD3ε, B2M, and RFXAP; (d) CD3ε, B2M, and RFXANK; (e) CD3ε, B2M, and HLA-DM; (f) CD3ε, B2M, and Ii chain; (g) CD3ε, TAP1, and CIITA; (h) CD3ε, TAP1, and RFX5; (i) CD3ε, TAP1, and RFXAP; (j) CD3ε, TAP1, and RFXANK; (k) CD3ε, TAP1, and HLA-DM; (1) CD3ε, TAP1, and Ii chain; (m) CD3ε, TAP2, and CIITA; (n) CD3ε, TAP2, and RFX5; (o) CD3ε, TAP2, and RFXAP; (p) CD3ε, TAP2, and RFXANK; (q) CD3ε, TAP2, and HLA-DM; (r) CD3ε, TAP2, and Ii chain; (s) CD3ε, NLRC5, and CIITA; (t) CD3ε, NLRC5, and RFX5; (u) CD3ε, NLRC5, and RFXAP; (v) CD3ε, NLRC5, and RFXANK; (w) CD3ε, NLRC5, and HLA-DM; (x) CD3ε, NLRC5, and Ii chain; (y) CD3ε, TAPBP, and CIITA; (z) CD3ε, TAPBP, and RFX5; (aa) CD3ε, TAPBP, and RFXAP; (bb) CD3ε, TAPBP, and RFXANK; (cc) CD3ε, TAPBP, and HLA-DM; or (dd) CD3ε, TAPBP, and Ii chain.

In some embodiments, the one or more nucleic acids introduced into the modified immune cell are capable of downregulating the gene expression of: (a) CD3δ, B2M, and CIITA; (b) CD3δ, B2M, and RFX5; (c) CD3δ, B2M, and RFXAP; (d) CD3δ, B2M, and RFXANK; (e) CD3δ, B2M, and HLA-DM; (f) CD3δ, B2M, and Ii chain; (g) CD3δ, TAP1, and CIITA; (h) CD3δ, TAP1, and RFX5; (i) CD3δ, TAP1, and RFXAP; (j) CD3δ, TAP1, and RFXANK; (k) CD3δ, TAP1, and HLA-DM; (1) CD3δ, TAP1, and Ii chain; (m) CD3δ, TAP2, and CIITA; (n) CD3δ, TAP2, and RFX5; (o) CD3δ, TAP2, and RFXAP; (p) CD3δ, TAP2, and RFXANK; (q) CD3δ, TAP2, and HLA-DM; (r) CD3δ, TAP2, and Ii chain; (s) CD3δ, NLRC5, and CIITA; (t) CD3δ, NLRC5, and RFX5; (u) CD3δ, NLRC5, and RFXAP; (v) CD3δ, NLRC5, and RFXANK; (w) CD3δ, NLRC5, and HLA-DM; (x) CD3δ, NLRC5, and Ii chain; (y) CD3δ, TAPBP, and CIITA; (z) CD3δ, TAPBP, and RFX5; (aa) CD3δ, TAPBP, and RFXAP; (bb) CD3δ, TAPBP, and RFXANK; (cc) CD3δ, TAPBP, and HLA-DM; or (dd) CD3δ, TAPBP, and Ii chain.

In some embodiments, the one or more nucleic acids introduced into the modified immune cell are capable of downregulating the gene expression of: (a) CD3γ, B2M, and CIITA; (b) CD3γ, B2M, and RFX5; (c) CD3γ, B2M, and RFXAP; (d) CD3γ, B2M, and RFXANK; (e) CD3γ, B2M, and HLA-DM; (f) CD3γ, B2M, and Ii chain; (g) CD3γ, TAP1, and CIITA; (h) CD3γ, TAP1, and RFX5; (i) CD3γ, TAP1, and RFXAP; (j) CD3γ, TAP1, and RFXANK; (k) CD3γ, TAP1, and HLA-DM; (1) CD3γ, TAP1, and Ii chain; (m) CD3γ, TAP2, and CIITA; (n) CD3γ, TAP2, and RFX5; (o) CD3γ, TAP2, and RFXAP; (p) CD3γ, TAP2, and RFXANK; (q) CD3γ, TAP2, and HLA-DM; (r) CD3γ, TAP2, and Ii chain; (s) CD3γ, NLRC5, and CIITA; (t) CD3γ, NLRC5, and RFX5; (u) CD3γ, NLRC5, and RFXAP; (v) CD3γ, NLRC5, and RFXANK; (w) CD3γ, NLRC5, and HLA-DM; (x) CD3γ, NLRC5, and Ii chain; (y) CD3γ, TAPBP, and CIITA; (z) CD3γ, TAPBP, and RFX5; (aa) CD3γ, TAPBP, and RFXAP; (bb) CD3γ, TAPBP, and RFXANK; (cc) CD3γ, TAPBP, and HLA-DM; or (dd) CD3γ, TAPBP, and Ii chain.

In some embodiments, the immune cell to be modified is selected from the group consisting of a T cell, a natural killer cell (NK cell), a natural killer T cell, a lymphoid progenitor cell, a hematopoietic stem cell, a stem cell, a macrophage, and a dendritic cell. In some embodiments, the immune cell to be modified the immune cell is a CD4+ T cell or a CD8+ T cell. In some embodiments, the immune cell to be modified is an allogeneic T cell or autologous T cell.

In some embodiments, the nucleic acids are introduced into the immune cell by viral transduction. In some embodiments, the viral transduction comprises contacting the immune cell with a viral vector comprising the one or more nucleic acids. In some embodiments, the viral vector is selected from the group consisting of a retroviral vector, sendai viral vectors, adenoviral vectors, adeno-associated virus vectors, and lentiviral vectors.

In some embodiments, each of the one or more nucleic acids that are capable of downregulating expression of one or more endogenous immune genes comprises a gene editing system selected from the group consisting of: (a) a CRISPR-associated (Cas) (CRISPR-CAs) endonuclease system and a guide RNA; (b) a TALEN gene editing system, a zinc finger nuclease (ZFN) gene editing system, a meganuclease gene editing system, or a mega-TALEN gene editing system; and (c) a gene silencing system selected from antisense RNA, antigomer RNA, RNAi, siRNA, or shRNA.

In some embodiments, the Cas endonuclease comprises Cas3, Cas4, Cas8a, Cas8b, Cas9, Cas10, Cas10d, Cas12a, Cas12b, Cas12d, Cas12e, Cas12f, Cas12g, Cas12h, Cas12i, Cas13, Cas14, CasX, Cse1, Csy1, Csn2, Cpf1, C2c1, Csm2, Cmr5, Fok1, S. pyogenes Cas9, Staphylococcus aureus Cas9, MAD7 nuclease (a type V CRISPR nuclease), or any combination thereof. In some embodiments, the CRISPR-Cas system comprises an pAd5/F35-CRISPR vector.

In some embodiments, the guide RNA of the CRISPR-Cas system comprises a guide sequence that is complementary with a sequence within the one or more gene loci each encoding the immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain).

In some embodiments, the guide RNA introduced into the modified immune cell is complementary with a sequence within one or more exons of CD3δ, CD3ε, or CD3γ. In some embodiments, the guide RNA is complementary with a sequence within exon 1 of CD3δ, CD3ε, or CD3γ.

In some embodiments, the guide RNA introduced into the modified immune cell is complementary with a sequence within the CD3δ gene locus and the guide RNA comprises a nucleic acid sequence encoded by SEQ ID NO: 53. In some embodiments, the guide RNA introduced into the modified immune cell is complementary with a sequence within the CD3ε gene locus and the guide RNA comprises a nucleic acid sequence encoded by SEQ ID NO: 52. In some embodiments, the guide RNA introduced into the modified immune cell is complementary with a sequence within the CD3γ gene locus and the guide RNA comprises a nucleic acid sequence encoded by SEQ ID NO: 54. In some embodiments, the guide RNA introduced into the modified immune cell is complementary with a sequence within the B2M gene locus and the guide RNA comprises a nucleic acid sequence encoded by SEQ ID NO: 55. In some embodiments, the guide RNA introduced into the modified immune cell is complementary with a sequence within the CIITA gene locus and the guide RNA comprises a nucleic acid sequence encoded by SEQ ID NO: 61. In some embodiments, the guide RNA introduced into the modified immune cell is complementary with a sequence within the TAP1 gene locus and the guide RNA comprises a nucleic acid sequence encoded by SEQ ID NO: 56. In some embodiments, the guide RNA introduced into the modified immune cell is complementary with a sequence within the TAP2 gene locus and the guide RNA comprises a nucleic acid sequence encoded by SEQ ID NO: 57. In some embodiments, the guide RNA introduced into the modified immune cell is complementary with a sequence within the TAPBP gene locus and the guide RNA comprises a nucleic acid sequence encoded by SEQ ID NO: 58, SEQ ID NO: 59, or any combination thereof. In some embodiments, the guide RNA introduced into the modified immune cell is complementary with a sequence within the NLRC5 gene locus and the guide RNA comprises a nucleic acid sequence encoded by SEQ ID NO: 60. In some embodiments, the guide RNA introduced into the modified immune cell is complementary with a sequence within the HLA-DM gene locus and the guide RNA comprises a nucleic acid sequence encoded by SEQ ID NO: 62. In some embodiments, the guide RNA introduced into the modified immune cell is complementary with a sequence within the RFX5 gene locus and the guide RNA comprises a nucleic acid sequence encoded by SEQ ID NO: 63, SEQ ID NO: 64, or any combination thereof. In some embodiments, the guide RNA introduced into the modified immune cell is complementary with a sequence within the RFXANK gene locus and the guide RNA comprises a nucleic acid sequence encoded by SEQ ID NO: 65. In some embodiments, the guide RNA introduced into the modified immune cell is complementary with a sequence within the RFXAP gene locus and the guide RNA comprises a nucleic acid sequence encoded by SEQ ID NO: 66. In some embodiments, the guide RNA introduced into the modified immune cell is complementary with a sequence within the Ii Chain gene locus and the guide RNA comprises a nucleic acid sequence encoded by SEQ ID NO: 67, SEQ ID NO: 68, or any combination thereof.

In some embodiments, the modified immune cell generated by the disclosed method exerts a reduced immune response in a subject when the modified immune cell is administered to a subject, as compared to the immune response exerted by an unmodified immune cell administered to the same subject.

In some embodiment, the modified immune cell the modified immune cell generated by the disclosed method exerts a reduced immune response in a subject when the immune cell is administered to the subject, as compared to the immune response exerted by an immune cell comprising one or more nucleic acids capable of downregulating the gene expression of TRAC, TRBC, B2M, and CIITA. In some embodiments, the reduced GvHD response by the modified immune cell generated by the disclosed method is compared to an equivalent immune cell without the deletion and/or insertion in one or more gene loci, or an immune cell comprising the deletion and/or insertion in TRAC, TRBC, B2M, and CIITA.

In some embodiments, the immune response is a graft-versus-host disease (GvHD) response. In some embodiments, the reduced GvHD response is elicited against an HLA-I mismatched cell or against an HLA-II mismatched cell. In some embodiments, the GvHD response elicited by a modified immune cell generated by the disclosed method is reduced by about 10% or more, about 20% or more, about 30% or more, about 40% or more, about 50% or more, about 60% or more, about 70% or more, about 80% or more, about 90% or more, or about 95% or more. In some embodiments, the GvHD response elicited by a modified immune cell generated by the disclosed method is reduced by about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 20-fold or more, about 30-fold or more, about 50-fold or more, about 100-fold or more, about 150-fold or more, or about 200-fold or more.

In some embodiments, the exogenous nucleic acid introduced into the immune cell encodes a chimeric antigen receptor (CAR). In some embodiments, the CAR comprises an antigen binding domain, a hinge domain, a transmembrane domain, a costimulatory signaling domain, and an intracellular signaling domain.

In some embodiments, the antigen binding domain targets a tumor antigen associated with a hematologic malignancy; and/or associated with a solid tumor. In some embodiments, the antigen binding domain targets a tumor antigen selected from the group consisting of ROR1, mesothelin, c-Met, PSMA, PSCA, Folate receptor alpha, Folate receptor beta, EGFR, EGFRvIII, GPC2, GPC2, Mucin 1 (MUC1), Tn antigen ((Tn Ag) or (GalNAca-Ser/Thr)), TnMUC1, GDNF family receptor alpha-4 (GFRa4), fibroblast activation protein (FAP), and Interleukin-13 receptor subunit alpha-2 (IL-13Ra2 or CD213A2).

In some embodiments, the CAR introduced into the modified immune cell comprises: (a) a PSMA antigen binding domain (e.g. SEQ ID NOs: 73 or 74), a CD2 costimulatory domain, and a CD3 zeta intracellular signaling domain; or (b) a mesothelin antigen binding domain (e.g., SEQ ID NO: 75), a 4-1BB costimulatory domain, and a CD3 zeta signaling domain; or (c) a TnMUC1 antigen binding domain, a CD2 costimulatory domain, and a CD3 zeta signaling domain. In some embodiments, the TnMUC1 CAR comprises an amino acid sequence set forth in SEQ ID NO: 70, and the mesothelin CAR comprises an amino acid sequence set forth in SEQ ID NO: 71 or SEQ ID NO: 72. In some embodiments, the TnMUC1 CAR is encoded by a nucleic acid sequence set forth in SEQ ID NO: 69.

In one aspect of the disclosure, the switch receptor introduced in the modified immune cell comprises: (a) an extracellular domain of a signaling protein associated with a negative signal selected from the group consisting of CTLA4, PD-1, VISG3, VSIG8, TGFβRII, BTLA, and TIM-3, (b) a transmembrane domain, and (c) an intracellular domain of a signaling protein associated with a positive signal selected from the group consisting of CD28, 4-1BB, IL12Rβ1, IL12Rβ2, CD2, ICOS, and CD27.

In some embodiments, the switch receptor is selected from the group consisting of PD-1-CD28, PD-1^{A132L}-CD28, PD-1-CD27, PD-1^{A132L}-CD27, PD-1-4-1BB, PD-1^{A132L}-4-1BB, PD-1-ICOS, PD-1^{A132L}-ICOS, PD-1-IL12Rβ1, PD-1^{A132L}-IL12Rβ1, PD-1-IL12Rβ2, PD-1^{A132L}-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2.

In some embodiments, the dominant negative receptor introduced into the modified immune cell comprises: (a) a truncated variant of a wild-type protein associated with a negative signal, or (b) a variant of a wild-type protein associated with a negative signal comprising an extracellular domain, a transmembrane domain, and substantially lacking an intracellular signaling domain; or (c) an extracellular domain of a signaling protein associated with a negative signal, and a transmembrane domain. In some embodiments, the dominant negative receptor is PD1, VSIG3, VSIG8, or TGFβR dominant negative receptor.

In some embodiments, the transmembrane domain of the switch receptor is selected from a transmembrane domain of a protein selected from the group consisting of CTLA4, PD-1, BTLA, TGFβRII, BTLA, TIM-3, CD28, 4-1BB, IL12Rβ1, IL12Rβ2, CD2, ICOS, and CD27. In some embodiments, the transmembrane domain of the switch receptor is selected from the transmembrane of the protein associated with a negative signal or the transmembrane domain of the protein associated with the negative signal.

One aspect of the present disclosure provides expanding the modified immune cell. In some embodiments, expanding the modified immune cell comprises culturing the T cell with a factor selected from the group consisting of flt3-L, IL-1, IL-3, IL-2, IL-7, IL-15, IL-18, IL-21, TGFbeta, IL-10, and c-kit ligand. One aspect of the present disclosure provides further introducing a polypeptide and/or a nucleic acid encoding Klf4, Oct3/4 and Sox2 in the immune cell to induce pluripotency of the immune cell.

In some embodiments, the immune cell is obtained from a blood sample, a whole blood sample, a peripheral blood mononuclear cell (PBMC) sample, or an apheresis sample. In some embodiments, the apheresis sample is a cryopreserved sample. In some embodiments, the apheresis sample is a fresh sample. In some embodiments, the immune cell is obtained from a human subject.

One aspect of the present disclosure provides a population of modified immune cells obtained by the method of any one of the preceding embodiments. In some embodiments, the composition comprises the modified immune cell of any one of the preceding embodiments. In some embodiments, the composition comprises a population of modified immune cells generated by the methods disclosed in any one of the preceding embodiments and pharmaceutically acceptable carrier or excipient.

One aspect of the present disclosure provide a method of treating a disease or condition associated with enhanced immunity in a subject comprising administering an effective amount of the composition disclosed in any one of the preceding embodiments to a subject in need thereof. In some embodiments, the condition is a cancer. In some embodiments, the cancer is selected from the group consisting of breast cancer, prostate cancer, ovarian cancer, cervical cancer, skin cancer, pancreatic cancer, colorectal cancer, renal cancer, liver cancer, brain cancer, lymphoma, leukemia, lung cancer, and any combination thereof. In some embodiments, the cancer is a solid tumor, or a hematologic malignancy. In some embodiments, a method of treating a cancer comprises administering to a subject the modified immune cells of in any one of the preceding embodiments, the population of modified T cells of any one of the preceding embodiments, or the composition of any one of the preceding embodiments.

One aspect of the present disclosure provides a method for stimulating a T cell-mediated immune response to a target cell or tissue in a subject comprising administering to a subject an effective amount of a pharmaceutical composition comprising the modified immune cell of any one of the preceding embodiments, the population of modified immune cells of any one of the preceding embodiments, or the composition of any one of the preceding embodiments.

One aspect of the present disclosure provide a kit comprising the modified immune cells of any one of the preceding embodiments, the population of modified T cells of any one of the preceding embodiments, or the composition of any one of the preceding embodiments, optionally comprising an instruction for use.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

The patent or application file contains at least one drawing executed in color. Copies of this patent or patent application publication with color drawing(s) will be provided by the Office upon request and payment of necessary fee.

FIG. 1 shows a schematic representation of the human T-cell receptor (TCR)-CD3 complex, comprising the variable TCR-alpha chain (TCR-α, TRAC), and TCR-beta chain (TCR-β, TRBC) coupled to three dimeric modules CD3δ/CD3ε, CD3γ/CD3ε, and CD3ζ/CD3ζ. The CD3δ/CD3ε, and CD3γ/CD3ε modules are the subject of the present disclosure.

FIGS. 2A-2C show bar graphs illustrating the TCR-α and TCR-β chains disruption efficiency on human T cells, as measured by flow cytometry following the targeted disruption of CD3δ (FIG. 2A), CD3ε (FIG. 2B), and CD3γ (FIG. 2C) genes using a CRISPR/Cas system.

FIG. 3 shows a graph illustrating the expansion of allogeneic CART-cells generated using the strategy of FIG. 1, and illustrates the number of population doublings over a ten-day period. The tested allogeneic CART cells are engineered T cells comprising TRAC knockout (TRAC KO), CD3δ knockout (D1 KO), CD3γ knockout (G4 KO), and CD3ε knockout (E4 KO).

FIGS. 4A and 4B show flow cytometry results comparing CRISPR-mediated downregulation of TCR-α chain (TRAC) knockout, CD3δ knockout (D1 KO), CD3γ knockout (G4 KO), and CD3ε knockout (E4 KO). FIG. 4A shows that CD3ε knockout (E4 KO) is a better target for T cell receptor knockout, as measured by surface expression of the TCR-α/β chain. FIG. 4B shows that allogeneic CART cells comprising CD3ε knockout (E4 KO) had higher transduction efficiency and were functionally better than CART cells comprising, for example CD3γ or CD3δ knockout; a PSMA CART cell embodiment is illustrated.

FIG. 5 shows a graph demonstrating the tumor killing capacity of allogeneic PSMA CART cells comprising the TCR-α chain (TRAC) knockout, CD3δ knockout (D1), CD3ε knockout (E4), and CD3γ knockout (G4); and illustrating that the PSMA E4 allogeneic CART cells have the best killing capacity. Target cells were PC3 cells.

FIGS. 6A-6D show CRISPR-Cas activity illustrated with the T7 endonuclease mismatch detection assay (T7E1). FIG. 6A show a representative gel electrophoresis image of T7E1-treated PCR products amplified from the sites of three different CRISPR-Cas C2TA (CIITA) gene using three different gRNA. FIGS. 6B-6D shows electropherograms generated by Agilent Bioanalyzer electropherogram of the T7E1 endonuclease assay demonstrating the CRISPR-Cas editing efficiency.

FIGS. 7A-D show Agilent Bioanalyzer electropherograms and gel electrophoresis of control and T7E1 treated PCR illustrating C2TA (CIITA) CRISPR editing efficiency result. In particular, FIG. 7A shows overall results for sample C2TA-1-PCR, FIG. 7B shows overall results for sample C2TA-1-T7E1, FIG. 7C shows overall results for sample C2TA-2-PCR, and FIG. 7D shows overall results for sample C2TA-2-T7E1.

FIG. 8 shows a graph illustrating the result of mixed lymphocyte reaction (MLR) assay; and demonstrating the viability of control T cells (2^{nd} donor), allogeneic PSMA CART cells alone or in co-culture; and illustrating that T cells from a 2^{nd} (irrelevant) donor do not proliferate in response to allogeneic PSMA CART cells in co-culture despite the presence of an HLA mismatch. Allogeneic CART cells comprise a PSMA CAR and a CRISPR edited TRAC/B2M/C2TA gRNAs.

FIG. 9 shows a table illustrating a novel allogeneic CART strategy of the present disclosure involving the knockout (KO) of alternative T cell receptor subunits (CD3δ, CD3γ, and CD3ε) and additional critical genes in the antigen processing and presentation pathways. In particular, 15 gene targets were selected and each was tested with multiple guide RNAs (gRNAs) using the CRISPR-associated (Cas) (CRISPR-Cas) endonuclease system.

### DETAILED DESCRIPTION

### I. Overview

The T cell receptor (TCR) complex is a large multi-subunit complex composed of at least eight polypeptide subunits (TCRαβ, CD3εγ, CD3εδ, and CD3ζζ). The TCRαβ heterodimer is the ligand-binding subunit responsible for recognizing antigens bound to major histocompatibility complex class I and class II molecules. CD3ε, CD3γ, CD3δ, and CD3ζ are organized as dimers to form three dimeric modules CD3δ/CD3ε, CD3γ/CD3ε, and CD3ζ/CD3ζ that transduce the signal generated by the TCRαβ heterodimer. To date, the GvHD- and/or HvHD-avoidance strategy has been the generation of allogeneic T cells that comprises the downregulation of the TCRα chain through targeted disruption of the TRAC gene locus. Prior to the present disclosure, the modulatory role of CD3ε, CD3γ, CD3δ, and CD3ζ in the promotion of GvHD and HvHD were not investigated as it was believed that disruption of CD3α/β was critical to successful generation of allogenic T cells. The present disclosure details a surprising discovery that the targeted disruption of CD3ε, CD3γ, CD3δ, and CD3ζ loci generated allogeneic CAR T cells that were as efficient and/or better than CAR T cells comprising a targeted disruption of the TRAC locus. In the present disclosure, disruption of at least one of the CD3ε, CD3γ, CD3δ genes is preferred.

The present invention includes methods and compositions for generating a modified T cell by knocking down one or more endogenous T cell receptor complex gene expression and expressing either a chimeric antigen receptor (CAR), an engineered T cell receptor (TCR), a Killer cell immunoglobulin-like receptor (KIR), an antigen-binding polypeptide, a cell surface receptor ligand, a tumor antigen, a dominant negative receptor, a switch receptor, a chemokine, a chemokine receptor, a cytokine, or a cytokine receptor.

Thus, the present disclosure is based on the observation that modified immune cells comprising at least one of a genetically edited CD3δ gene, CD3ε gene, CD3ζ gene, and/or CD3γ gene of a T-cell receptor complex, combined with a chimeric antigen receptor (CAR), and/or a switch receptor, and/or an immune enhancing factor, are improved allogeneic T cells with enhanced fitness that demonstrate potent cytosolic activity against various cancer cell lines in vitro, as well as significant tumor eradication in vivo when compared to standard allogeneic T cells known in the art.

Adoptive immunotherapy offers exciting promise to cancer patients. However, several challenges currently exist relating to the manufacturing of CAR-T and TCR cells. These challenges impact the potential success of adoptive immunotherapy.

Despite approval and general success of autologous adoptive immunotherapies, the scalability and feasibility of such therapies present significant challenges. In particular, the widespread clinical application of adoptive immunotherapy is limited by the considerable economic constraints imposed by the personalized preparation of autologous CART-cells. Yet, a standardized adoptive immunotherapy in which allogeneic therapeutic cells are pre-manufactured, characterized in detail, and available for immediate administration to a broad range of patients remains a dangerous procedure with many possible complications, such as allogeneic T-cell responses. Allogeneic T-cell response is clinically manifested as graft-versus-host disease (GVHD) and/or Host-versus-Graft disease (HvGD, graft rejection). GvHD is caused by the attack of recipient tissues by infused allogeneic CAR-T cell mediated by alloreactive TCR on donor CAR cells. In particular, endogenous T-cell receptor alpha (TCRα; TRAC) and beta (TCRβ; TRBC) chains on infused T cells recognize major and minor histocompatibility antigens in the recipient, leading to (GvHD). Conversely, infused allogeneic CART cells may be rejected by the recipient T lymphocytes leading to HvGD.

Prior to the present disclosure, one approach to address Allogeneic T-cell response was to engineer allogeneic T cells using genome-editing techniques to abolish the expression of TCRα, TCRβ and/or one or more Major Histocompatibility (MHC) class I (MHC I) and/or MHC class II complexes in allogeneic donor T cells. Because the TCRαβ heterodimer is necessary for the assembly and activity of the entire TCR complex, knocking out the expression of either the TCR α or β chains prevents donor CAR T cells from recognizing host alloantigens, and thus GVHD. Furthermore, editing out MHC I in donor T cells conversely prevents recognition of these allogeneic T cells by T cells of the recipient and thus rejection of the graft. To date, deletion of the α chain through targeted disruption of the TRAC locus has been utilized as an GVHD-avoidance strategy mainly because the β chain is encoded by two TRBC genes (TRBC1 and TRBC2). As such knocking out the TRBC gene is potentially more complicated.

An optimal protocol to efficiently introduce Cas9/sgRNAs into T cells with minimal toxicity remains to be established. Furthermore, current techniques do not result in TCR knockout in 100% of CART cells. This is significant as successful generation of allogenic cells useful in CART therapy would benefit from 100% TCR knockout.

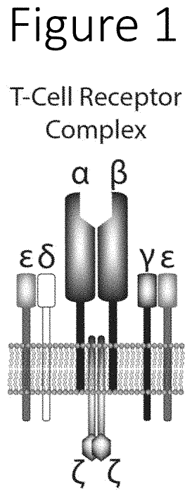

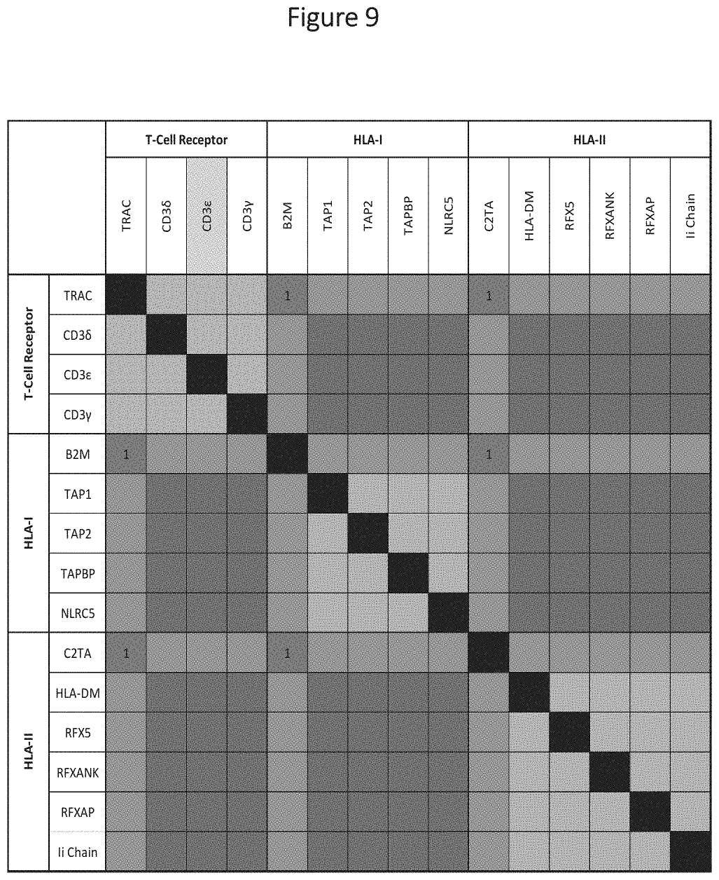

To address this issue, the present disclosure details disruption of the CD3ε gene, the CD3γ gene, and/or the CD3δ gene (see FIG. 1), both individually as well as combined with at least one other gene knockout, as detailed in FIG. 9 below (The CD3ζ gene can also be disrupted.) The disclosure also encompasses constructs and uses thereof where at least one of CD3ε, CD3γ, and/or CD3δ (and optionally the CD3ζ gene) is disrupted. The disruption of at least one of CD3ε, CD3γ, and/or CD3δ (and optionally the CD3ζ gene) can also be combined with disruption of one or more of CD3α and CD3β (e.g., knock out of the CD3ε gene and the CD3γ gene; knock out of the CD3ε gene and the CD3α gene; knock out of the CD3γ gene and the CD3δ gene, etc.). See e.g., Example 2 and FIG. 9, which illustrates a novel allogeneic CART strategy of the present invention involving the knockout (KO) of alternative T cell receptor subunits (CD3δ, CD3γ, and CD3ε) and additional critical genes in the antigen processing and presentation pathways.

It was surprisingly found that two or three (or more) disruptions interfered with T cell receptor expression, and successfully resulted in the downregulation of the TCR with minimal toxicity.

In addition, TRAC-negative T cells were shown to persist longer in tumors (Stadtmauer et. al., Science, 367(6481): eaba7365 (2020)), so it was surprising that focusing on disruption of CD3ε gene, CD3γ gene, CD3ζ gene, and/or CD3δ gene also produced T cells effective in adoptive immunotherapy (although as noted above the present disclosure also encompasses disruption of one or more of CD3ε, CD3γ, CD3ζ and/or CD3δ genes combined with disruption of CD3α and/or CD3β genes).

The present disclosure provides novel and alternative approaches for modulating the functional properties of T cells by alternatively tuning and modulating TCR signaling associated with allogeneic T cell responses. In particular, the present disclosure demonstrates that the downregulation of at least one of CD3ε gene, CD3γ gene, CD3ζ and/or CD3δ gene, either alone or in combination with one or more additional TCR complex components, significantly reduced allogeneic T-cell responses, while preserving CART cells beneficial antitumor properties, thereby generating safe and effective CART cells. The advantages of these novel and alternative approaches are described in more detail below.

### II. Experimental Results

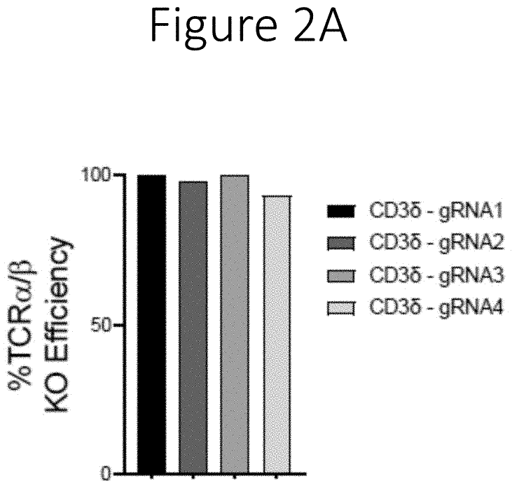

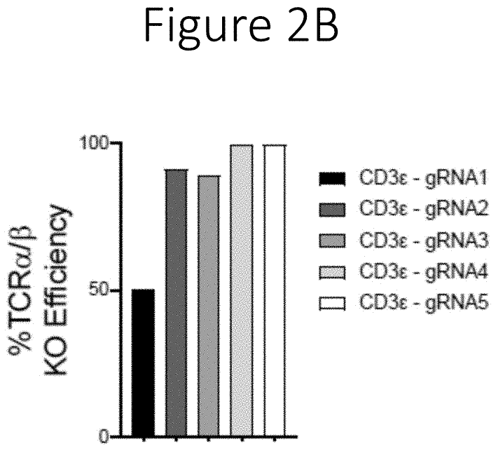

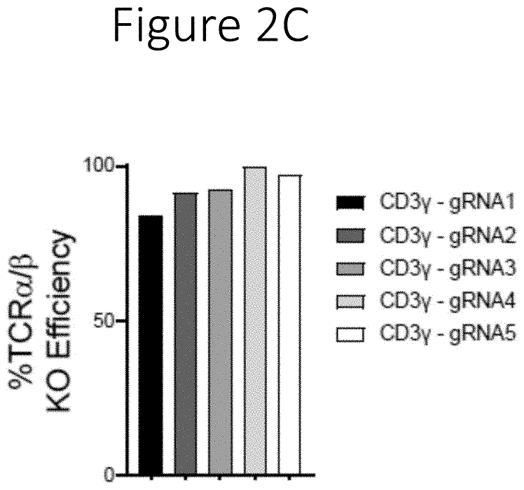

The percentage of TCR-α and TCR-β chains disruption efficiency on human T cells was measured by flow cytometry following the targeted disruption of CD3δ (FIG. 2A), CD3ε (FIG. 2B), and CD3γ (FIG. 2C) genes using a CRISPR/Cas system (see also Example 3 below). FIG. 2A shows the results following disruption of CD3δ using four different guide RNAs: gRNA1, gRNA2, gRNA3, and gRNA4. Targeted disruption of CD3δ using gRNA1 and gRNA3 guide RNAs in the CRISPR/Cas system resulted in 100% KO efficiency of TCR α/β, while use of gRNA2 and gRNA4 in the CRISPR/Cas system resulted in greater than about 90% KO efficiency of TCR α/β. Thus, use of gRNA1 and gRNA3 are preferred in a CRISPR/Cas system for disrupting CD3δ.

FIG. 2B shows the results following the targeted disruption of CD3ε using five different guide RNAs: gRNA1, gRNA2, gRNA3, gRNA4, and gRNA5 Disruption of CD3ε using gRNA4 and gRNA5 guide RNAs in the CRISPR/Cas system resulted in 100% KO efficiency of TCR α/β, while use of gRNA1 resulted in only about a 50% KO efficiency of TCR α/β, and finally use of guide gRNA2 and gRNA3 in the CRISPR/Cas system resulted in greater than about 90% KO efficiency of TCR α/β. Thus, use of use of gRNA4 and gRNA5 guide RNAs are preferred in a CRISPR/Cas system for disrupting CD3ε.

FIG. 2C shows the results following the targeted disruption of CD3γ using five different guide RNAs: gRNA1, gRNA2, gRNA3, gRNA4, and gRNA5 Disruption of CD3ε using gRNA4 and guide RNAs in the CRISPR/Cas system resulted in 100% KO efficiency of TCR α/β, while use of gRNA5 resulted in a greater than about 95% KO efficiency of TCR α/β, and finally use of gRNA1, gRNA2 and gRNA3 guide RNAs in the CRISPR/Cas system resulted in less favorable KO efficiency of TCR α/β. Thus, use of use of gRNA4 guide RNA are preferred in a CRISPR/Cas system for disrupting CD3γ.

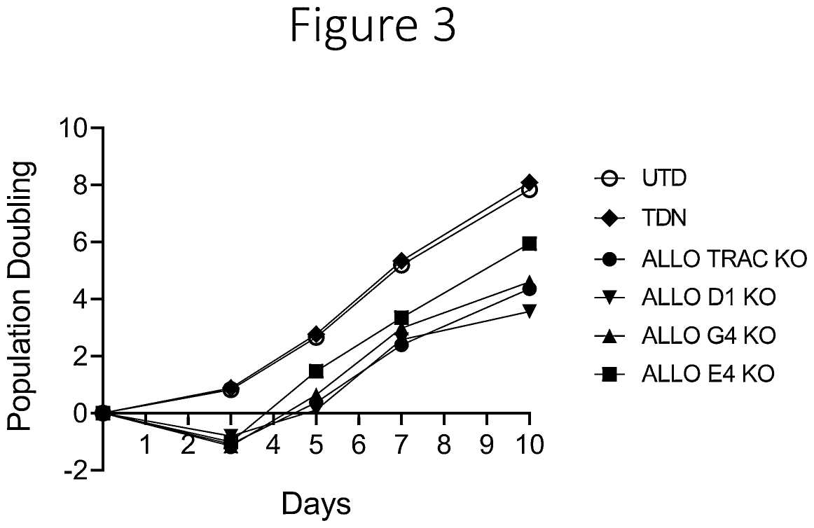

In another experiment with details provided in Example 4 below, the expansion of different constructs of allogenic CART-cells over a 10 day period was evaluated. This data is important as if the modified immune cells do not expand, then a sufficient number of cells will not be generated for successful therapy. The different constructs tested included allogeneic CART cells comprising: (1) TRAC knockout (ALLO (TRAC KO) on FIG. 3) (e.g., the knockout used prior to the present disclosure), (2) CD3δ knockout (ALLO (D1 KO) on FIG. 3), (3) CD3γ knockout (ALLO (G4 KO) on FIG. 3), and (4) CD3ε knockout (ALLO (E4 KO) on FIG. 3). The percentage of the cell population doubling is shown on the Y axis while the number of days is shown on the X axis. The results detailed in FIG. 3 show that all modified cells produced about 4× doubling, or more, over a 9 day period of time, thus demonstrating that the modified cells produce a useful quantity of material useful for immunotherapy.

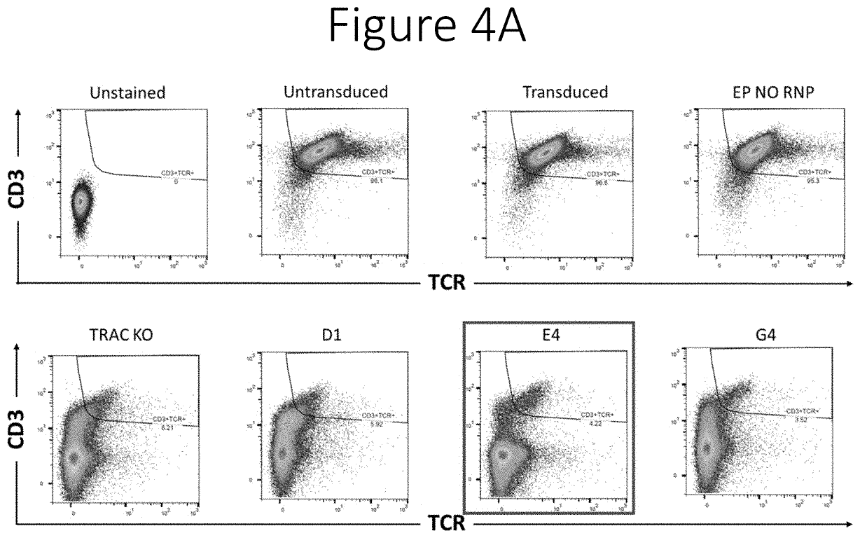

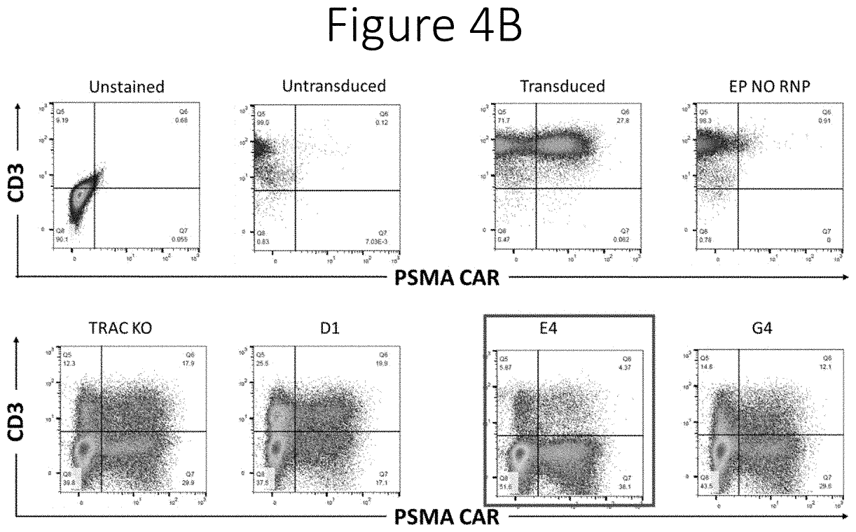

Example 5 evaluated several different knockout constructs to evaluate surface expression of the TCR-α/β chain. In particular, FIGS. 4A and 4B show flow cytometry results comparing CRISPR-mediated downregulation of TCR-α chain (TRAC) knockout (e.g., the construct used prior to the present disclosure), CD3δ knockout (D1 KO), CD3γ knockout (G4 KO), and CD3ε knockout (E4 KO). FIG. 4A shows that CD3ε knockout (E4 KO) was a better target for T cell receptor knockout, as measured by surface expression of the TCR-α/β chain. FIG. 4B shows that allogeneic CART cells comprising CD3ε knockout (E4 KO) had higher transduction efficiency and were functionally better than CART cells comprising, for example CD3γ or CD3δ knockout; a PSMA CART cell embodiment is illustrated. Furthermore, FIG. 4B shows that the majority of the CART cells were allogeneic (CD3 and TCR negative), which means that patients administered with CART cells of the present invention will be infused with a higher number of allo CART cells.

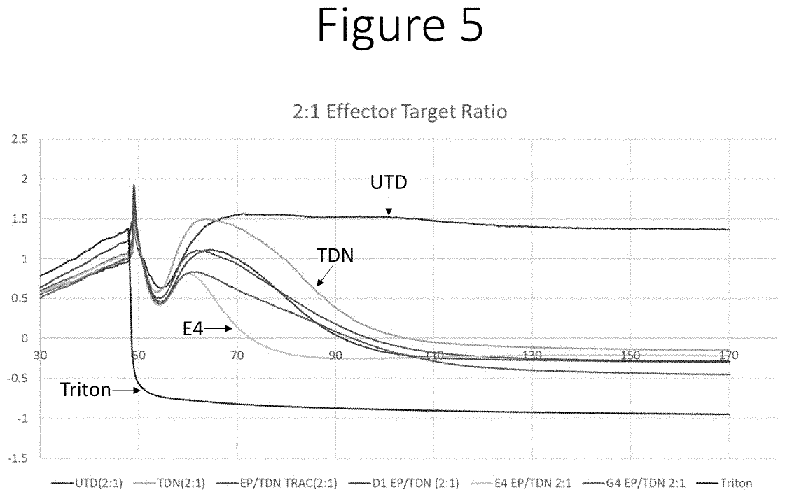

In a further experiment detailed in Example 6 below, the tumor killing capacity of different PSMA CART cell constructs was evaluated, as shown in FIG. 5. In particular, FIG. 5. shows a graph demonstrating the tumor killing capacity of allogeneic PSMA CART cells comprising the TCR-α chain (TRAC) knockout (e.g., the construct used prior to the present disclosure), CD3δ knockout (D1), CD3ε knockout (E4), and CD3γ knockout (G4). The results illustrate that the PSMA E4 allogeneic CART cells have the best killing capacity. Target cells were PC3 cells, which is a human prostate cancer cell line. E4 was unexpectedly found to be more potent than D1 and G4. Allo CART cells made by targeting the CD3ε molecule were more potent (e.g. kill tumor cells much faster) when compared to the other allo CART cells evaluated. The higher potency of CD3ε knockout (E4) CART cells means that tumor cells targeted by these CART cells will be eradicated much faster when compared to TCR-α chain (TRAC) knockout CART cells, CD3δ knockout (D1) CART cells, and/or CD3γ knockout (G4) CART cells. And time is of the essence for our allogeneic strategy to be successful in patients.

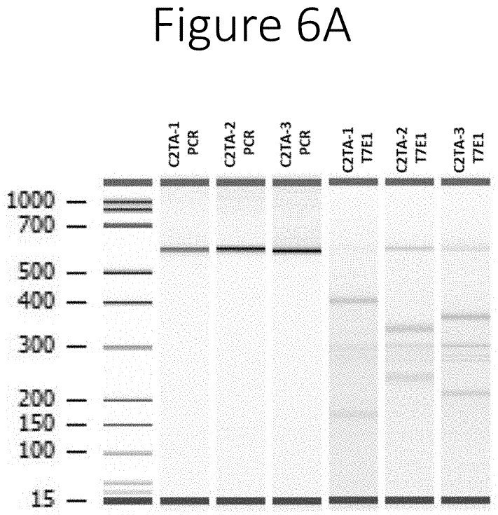

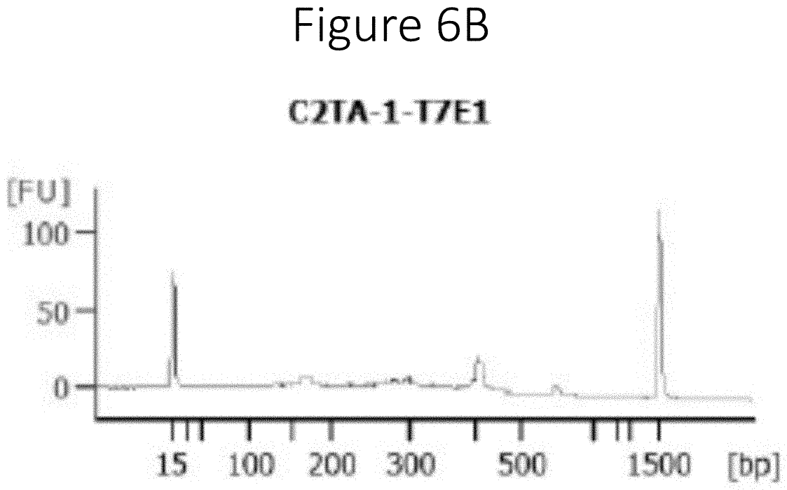

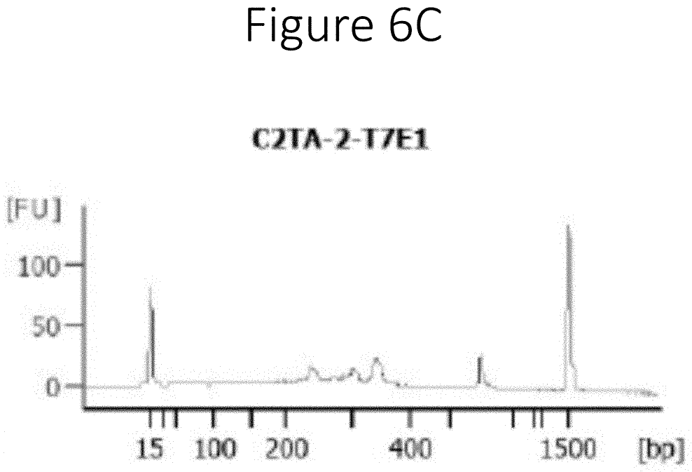

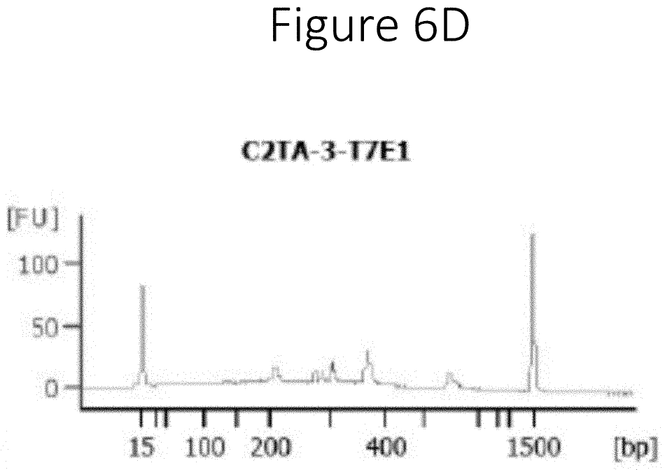

Example 7 describes evaluation of the effectiveness of the CRISPR-Cas methodology in effecting a knockout of the target gene. In particular, FIGS. 6A-6D show CRISPR-Cas activity illustrated with the T7 endonuclease mismatch detection assay (T7E1). FIG. 6A show a representative gel electrophoresis image of T7E1-treated PCR products amplified from the sites of three different CRISPR-Cas C2TA (CIITA) gene using three different gRNA. FIGS. 6B-6D shows electropherograms generated by Agilent Bioanalyzer electropherogram of the T7E1 endonuclease assay demonstrating the CRISPR-Cas editing efficiency.

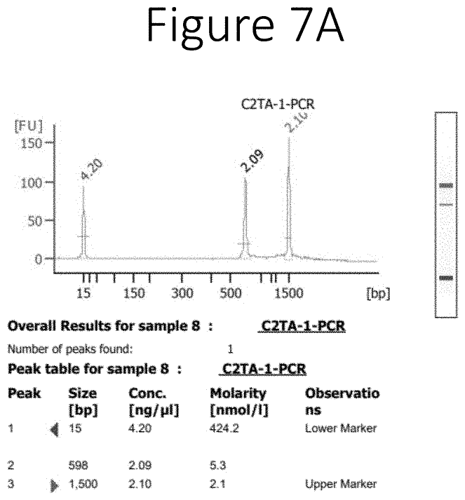

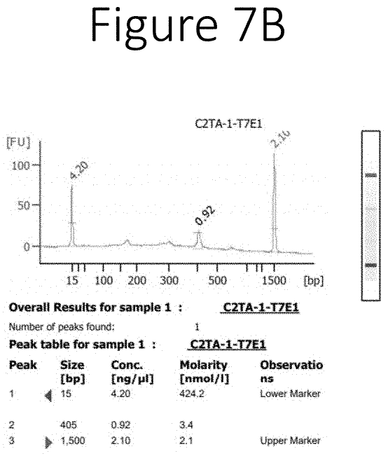

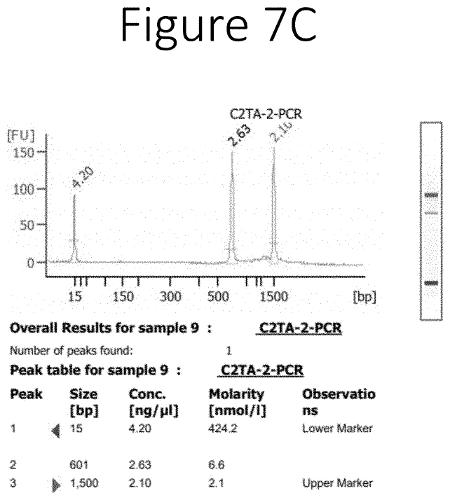

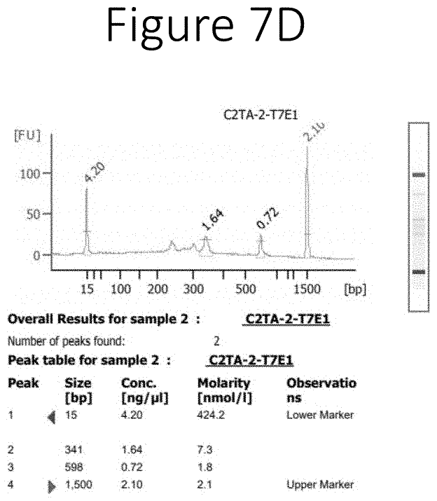

In addition, FIGS. 7A-D show Agilent Bioanalyzer electropherograms and gel electrophoresis of control and T7E1 treated PCR illustrating C2TA (CIITA) CRISPR editing efficiency result. FIG. 7A shows overall results for sample C2TA-1-PCR, FIG. 7B shows overall results for sample C2TA-1-T7E1, FIG. 7C shows overall results for sample C2TA-2-PCR, and FIG. 7D shows overall results for sample C2TA-2-T7E1.

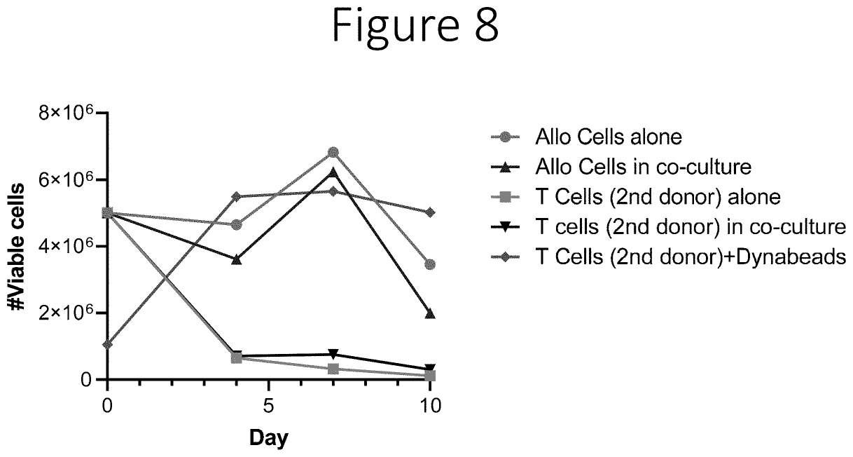

Finally, a mixed lymphocyte assay (MLA) was conducted. FIG. 8 shows a graph illustrating the result of mixed lymphocyte reaction (MLR) assay with Allo Cells alone, T cells (2^{nd} donor; T cells from a different donor) alone, Allo Cells in co-culture, and T cells (2^{nd} donor) in co-culture. In particular, recipient's T cells (T cells from a different donor) were co-cultured with allogeneic CART cells for 14 days, and proliferation of T cells were analyzed. The results demonstrate the viability of control T cells (2^{nd} donor), allogeneic PSMA CART cells alone or in co-culture, and show that “recipient's” T cells did not react (no proliferation) to the presence of allogeneic cells. Therefore, FIG. 8 shows that T cells from a 2^{nd} (irrelevant) donor did not proliferate in response to allogeneic PSMA CART cells in co-culture despite the presence of an HLA mismatch. Allogeneic CART comprises PSMA CART and CRISPR edited TRAC/B2M/C2TA gRNAs. Accordingly, allogeneic CART cells of the present invention will have a window of opportunity to kill tumor cells while being undetected by the recipient's immune system (i.e. T cells).

### III. Allogeneic T Cells

A. Downregulation of Endogenous Immune Proteins

One aspect of the present disclosure provides a modified immune cell that comprises (1) an insertion and/or deletion in one or more gene loci each encoding an endogenous immune protein; (2) an exogenous nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen. Accordingly, the modified cell of the present invention is genetically edited to disrupt the expression of any of the endogenous genes described herein. In some embodiments, a modified cell comprising an engineered TCR or CAR expression system of the present invention is genetically edited to disrupt the expression of one or more of the endogenous genes described herein. In some embodiments, where one or more of CD3δ, CD3ε, CD3γ, B2M, CIITA, TAP1, TAP2, TAPBP, NLRC5, HLA-DM, RFX5, RFXANK, RFXAP, and invariant chain (Ii Chain), are disrupted, an allogeneic T cell (i.e. universal immune cell) is produced. As used herein, the term “universal immune cell,” or “universal T cell” refers to an allogeneic immune cell or a T cell that is pre-modified/pre-manufactured for administration into any patient. In some embodiments, the modified immune cell of the present invention is an allogeneic T cell product with reduced or suppressed allogeneic T cell response. In some embodiments, the downregulation of the one or more gene loci of an endogenous immune protein reduces and/or eliminates GvHD and/or HvHD.

In some embodiments, the immune cell exerts a reduced immune response in a subject when the modified immune cell is administered to the subject, as compared to the immune response exerted by an unmodified immune cell administered to the same subject. In some embodiments, the immune cell of the present disclosure exerts a reduced immune response in a subject when the modified immune cell is administered to the subject, as compared to the immune response exerted by an immune cell comprising an insertion and/or deletion capable of downregulating the gene expression of TRAC, TRBC, B2M, and CIITA. In some embodiments, the immune response is a graft-versus-host disease (GvHD) or host-versus-graft disease (HvHD; graft rejection) response. In some embodiments, the reduced GvHD response is elicited against an HLA-I mismatched cell or against an HLA-II mismatched cell. In some embodiments, the GvHD response is reduced by about 10% or more, about 20% or more, about 30% or more, about 40% or more, about 50% or more, about 60% or more, about 70% or more, about 80% or more, about 90% or more, or about 95% or more. In some embodiments, the GvHD response is reduced by about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 20-fold or more, about 30-fold or more, about 50-fold or more, about 100-fold or more, about 150-fold or more, or about 200-fold or more. In some embodiments, the reduced GvHD response by the modified immune cell is compared to an equivalent immune cell without the deletion and/or insertion in one or more gene loci, or an immune cell comprising the deletion and/or insertion in TRAC, TCRP, B2M, and CIITA.

In some embodiments, the insertion and/or deletion in one or more gene loci is capable of downregulating gene expression of the one or more endogenous immune proteins loci. In some embodiments, the endogenous immune protein is one of the components of the TCR complex. In some embodiments, the endogenous immune protein is one or more of CD3ε CD3γ, CD3δ, and CD3ζ, which organize as heterodimers and form three dimeric modules CD3δ/CD3ε, CD3γ/CD3ε, and CD3ζ/CD3ζ that transduce a signal generated by the ligand binding to the TCRαβ heterodimer. In some embodiments, the ligand is an antigen-bound to major histocompatibility complex class I and class II (MHC-I; MHC-II) molecules, which is recognized by the TCRαβ heterodimer.

In some embodiments, the endogenous immune protein is a MHC class I (MHC-I) and/or a MHC class II (MHC-II) molecule. In some embodiments, the endogenous immune protein is B2M (Beta-2-microglobulin), CIITA (class II transactivator), TAP1 (ABC transporter associated with antigen processing 1; Transport1, ATP Binding Cassette Subfamily B Member), TAP2 (ABC transporter associated with antigen processing 2; Transport2, ATP Binding Cassette Subfamily B Member), TAPBP (TAP binding protein; Tapasin; TAP associated protein), NLRC5 (NLR Family CARD Domain Containing 5), HLA-DM, RFX5 (Regulatory Factor X5), RFXANK (Regulatory Factor X Associated Ankyrin Containing Protein), RFXAP (Regulatory Factor X Associated Protein), and invariant chain (Ii Chain).

Like TCR, MHC-I and MHC-II play essential roles in the activation of adaptive immune responses by presenting antigens to T lymphocytes. Humans have three major MHC-I loci (HLA-A, HLA-B, and HLA-C), which are vital to the detection and elimination of viruses, cancerous cells, and transplanted cells. In addition, there are three non-classical MHC-I molecules (HLA-E, HLA-F, and HLA-G), which have immune regulatory functions. Humans MHC-II molecules also comprises three loci (HLA-DP, HLA-DQ, and HLA-DR). Each of the MHC class I and II also comprises several regulatory proteins. MHC-I modulatory proteins include Beta-2-microglobulin (B2M), antigen-processing molecules such as TAP1, TAP2, TAPBP, and a transcriptional regulator, such as NLRCS. The Tap1, Tap2, and TAPBP are parts of the TAP transporter complex that is essential for loading peptide antigens onto the class I HLA complexes. Downregulation of the expression of any of B2M, NLRC5, Tap1, Tap2, and TAPBP results in the reduced cell surface expression of the MHC class I protein and impaired immune responses. In some embodiments, the endogenous immune protein contemplated by the present disclosure is a MHC-I gene selected from the group consisting of B2M, TAP1, TAP2, TAPBP, NLRC5, and a combination thereof.

MHC-II regulatory proteins include the transcriptional regulators CIITA, RFX5, RFXANK, RFXAP; and chaperone proteins involved in the formation and transport of MHC class II peptide, invariant chain (Ii Chain) and Human Leukocyte antigen DM (HLA-DM; HLA-DMA), HLA-DOA, and HLA-DOB. RFX5, RFXANK, RFXAP are subunits of a trimeric RFX DNA-binding complex that binds specifically to all MHC class II genes promoters to regulate their transcriptions. The Invariant chain (Ii) functions as an MHC class II chaperone that prevents peptide loading in the ER, stimulates exit from the ER, and modulates antigenic peptide loading. Akin to TAPBP, HLA-DM (e.g., HLA-DMA) assists in peptide loading of MHC-II molecules during intracellular trafficking. HLA-DM eliminates/prevents the display of weak-binding peptides to MHC-II proteins by guiding the T cell response to ‘immunodominant’ regions of antigens. In some embodiments, HLA-DM (e.g., HLA-DMA), promotes the elimination of potentially autoreactive T cells in the processing of self-proteins. In some embodiments, the endogenous immune protein contemplated by the present disclosure is a MHC-II gene selected from the group consisting of CIITA, HLA-DM, RFX5, RFXANK, RFXAP, invariant chain (Ii Chain), and a combination thereof.

Therefore, the efficient removal of the HLA barrier to reduce HvHD or GvHD can be accomplished by downregulating one or more of the following: (1) targeting the polymorphic MHC-I genes (HLA-A, -B, -C) and/or MHC-II genes (HLA-DP, -DQ, -DR); (2) targeting molecules that modulate the trafficking of all MHC-I molecules to the cell surface, such as B2M, or an MHC-I antigen-processing molecule such as TAP1, TAP2, or TAPBP; (3) targeting molecules that modulate MHC-II molecules trafficking, such as the invariant chain (Ii; or CD74)) or HLA-DM; and/or (4) targeting transcriptional regulators of MHC-I (NLRC5), or MHC-II expression (CIITA, RFX-5, RFXANK, RFX-AP).

In some embodiments, the endogenous immune protein whose gene expression is downregulated by the insertion and/or deletion is selected from the group consisting of CD3δ, CD3ε, CD3γ, B2M, CIITA, TAP1, TAP2, TAPBP, NLRC5, HLA-DM, RFX5, RFXANK, RFXAP, invariant chain (Ii Chain; CD74), or any combination thereof. In some embodiments, a modified immune cell (i.e a T cell) with downregulated gene expression of an endogenous immune protein selected from CD3δ, CD3ε, CD3γ, B2M, CIITA, TAP1, TAP2, TAPBP, NLRC5, HLA-DM, RFX5, RFXANK, RFXAP, invariant chain (Ii Chain; CD74), and a combination thereof has reduced immunogenicity in an allogeneic environment. In some embodiments, downregulating the gene expression of CD3δ, CD3ε, CD3γ, B2M, CIITA, TAP1, TAP2, TAPBP, NLRC5, HLA-DM, RFX5, RFXANK, RFXAP, invariant chain (Ii Chain; CD74), or any combination thereof removes surface presentation of alloantigens on the T cell that could cause HvHD and/or GvHD and/or prevents membrane expression of the T cell receptor.

In some embodiments, the modified immune cell used to generate an allogeneic T cell product comprises a triple knockout comprising the downregulation of one T cell receptor subunit, one HLA class I molecule; and one HLA class II molecule. In some embodiments, the T cell receptor subunit is selected from CD3δ, CD3ε, or CD3γ; the HLA class I molecule is selected from B2M, TAP1, TAP2, TAPBP, or NLRC5; and the HLA class II molecule selected from HLA-DM, RFX5, RFXANK, RFXAP, or invariant chain (Ii Chain). In some embodiments, the allogeneic T cell product comprises more than three endogenous immune protein knockouts. In such an embodiment, the T cell receptor subunit is selected from CD3δ, CD3ε, and/or CD3γ; the HLA class I molecule is selected from B2M, TAP1, TAP2, TAPBP, and/or NLRC5; and the HLA class II molecule is selected from HLA-DM, RFX5, RFXANK, RFXAP, and/or invariant chain (Ii Chain). In some embodiments, the modified immune cell for generating an allogeneic T cell product comprises one or more endogenous immune protein knockout comprising the downregulation of at least two T cell receptor subunits, at least two HLA class I molecules; and at least two HLA class II molecules. In some embodiments, the T cell receptor subunit is selected from at least two of CD3δ, CD3ε, and CD3γ; the HLA class I molecule is selected from at least two of B2M, TAP1, TAP2, TAPBP, and NLRC5; and the HLA class II molecule is selected from at least two HLA-DM, RFX5, RFXANK, RFXAP, and invariant chain (Ii Chain).

In some embodiments, the modified immune cell comprises an insertion and/or deletion that downregulates the CD3δ gene expression and the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof. In some embodiments, the modified immune cell comprises an insertion and/or deletion that downregulates a CD3ε gene expression and the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof. In some embodiments, the modified immune cell comprises an insertion and/or deletion that downregulates a CD3γ gene expression and the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof.

In some embodiments, when the gene expression of CD3δ, CD3ε, and/or CD3γ is downregulated, the surface expression of the T cell receptor alpha and beta is downregulated by at least about 50%, at least about 60%, at least about 70%, at least about 80%, at least about 90%, at least about 95%, at least about 99%, or at least about 100%. In some embodiments, the downregulation of CD3ε generates a higher T cell receptor Knockout efficiency when compared to the downregulation of CD3γ, and/or CD3δ. In some embodiments, the T cell receptor Knockout efficiency induced by the downregulation of CD3ε is higher or equal to the downregulation of TRAC.

In some embodiments, the modified immune cell used to generate an allogeneic T cell product comprises a triple knockout. In some embodiments, the modified immune comprises reduced or eliminated gene expression of (1) CD3ε, B2M, and CIITA; (2) CD3ε, B2M, and RFX5; (3) CD3ε, B2M, and RFXAP; (4) CD3ε, B2M, and RFXANK; (5) CD3ε, B2M, and HLA-DM; (6) CD3ε, B2M, and Ii chain; (7) CD3ε, TAP1, and CIITA; (8) CD3 c, TAP1, and RFX5; (9) CD3ε, TAP1, and RFXAP; (10) CD3ε, TAP1, and RFXANK; (11) CD3ε, TAP1, and HLA-DM; (12) CD3ε, TAP1, and Ii chain; (13) CD3ε, TAP2, and CIITA; (14) CD3ε, TAP2, and RFX5; (15) CD3ε, TAP2, and RFXAP; (16) CD3ε, TAP2, and RFXANK; (17) CD3ε, TAP2, and HLA-DM; (18) CD3ε, TAP2, and Ii chain; (19) CD3ε, NLRC5, and CIITA; (20) CD3ε, NLRC5, and RFX5; (21) CD3ε, NLRC5, and RFXAP; (22) CD3ε, NLRC5, and RFXANK; (23) CD3ε, NLRC5, and HLA-DM; (24) CD3ε, NLRC5, and Ii chain; (25) CD3ε, TAPBP, and CIITA; (26) CD3ε, TAPBP, and RFX5; (27) CD3ε, TAPBP, and RFXAP; (28) CD3ε, TAPBP, and RFXANK; (29) CD3ε, TAPBP, and HLA-DM; or (30) CD3ε, TAPBP, and Ii chain.

In some embodiments, the modified immune comprises reduced or eliminated gene expression of: (1) CD3δ, B2M, and CIITA; (2) CD3δ, B2M, and RFX5; (3) CD3δ, B2M, and RFXAP; (4) CD3δ, B2M, and RFXANK; (5) CD3δ, B2M, and HLA-DM; (6) CD3δ, B2M, and Ii chain; (7) CD3δ, TAP1, and CIITA; (8) CD3δ, TAP1, and RFX5; (9) CD3δ, TAP1, and RFXAP; (10) CD3δ, TAP1, and RFXANK; (11) CD3δ, TAP1, and HLA-DM; (12) CD3δ, TAP1, and Ii chain; (13) CD3δ, TAP2, and CIITA; (14) CD3δ, TAP2, and RFX5; (15) CD3δ, TAP2, and RFXAP; (16) CD3δ, TAP2, and RFXANK; (17) CD3δ, TAP2, and HLA-DM; (18) CD3δ, TAP2, and Ii chain; (19) CD3δ, NLRC5, and CIITA; (20) CD3δ, NLRC5, and RFX5; (21) CD3δ, NLRC5, and RFXAP; (22) CD3δ, NLRC5, and RFXANK; (23) CD3δ, NLRC5, and HLA-DM; (24) CD3δ, NLRC5, and Ii chain; (25) CD3δ, TAPBP, and CIITA; (26) CD3δ, TAPBP, and RFX5; (27) CD3δ, TAPBP, and RFXAP; (28) CD3δ, TAPBP, and RFXANK; (29) CD3δ, TAPBP, and HLA-DM; or (30) CD3δ, TAPBP, and Ii chain.

In some embodiments, the modified immune comprises reduced or eliminated gene expression of: (1) CD3γ, B2M, and CIITA; (2) CD3γ, B2M, and RFX5; (3) CD3γ, B2M, and RFXAP; (4) CD3γ, B2M, and RFXANK; (5) CD3γ, B2M, and HLA-DM; (6) CD3γ, B2M, and Ii chain; (7) CD3γ, TAP1, and CIITA; (8) CD3γ, TAP1, and RFX5; (9) CD3γ, TAP1, and RFXAP; (10) CD3γ, TAP1, and RFXANK; (11) CD3γ, TAP1, and HLA-DM; (12) CD3γ, TAP1, and Ii chain; (13) CD3γ, TAP2, and CIITA; (14) CD3γ, TAP2, and RFX5; (15) CD3γ, TAP2, and RFXAP; (16) CD3γ, TAP2, and RFXANK; (17) CD3γ, TAP2, and HLA-DM; (18) CD3γ, TAP2, and Ii chain; (19) CD3γ, NLRC5, and CIITA; (20) CD3γ, NLRC5, and RFX5; (21) CD3γ, NLRC5, and RFXAP; (22) CD3γ, NLRC5, and RFXANK; (23) CD3γ, NLRC5, and HLA-DM; (24) CD3γ, NLRC5, and Ii chain; (25) CD3γ, TAPBP, and CIITA; (26) CD3γ, TAPBP, and RFX5; (27) CD3γ, TAPBP, and RFXAP; (28) CD3γ, TAPBP, and RFXANK; (29) CD3γ, TAPBP, and HLA-DM; or (30) CD3γ, TAPBP, and Ii chain

In some embodiments, the modified immune cell of the present disclosure is a T cell, a natural killer cell (NK cell), a natural killer T cell (NKT), a lymphoid progenitor cell, a hematopoietic stem cell, a stem cell, a macrophage, or a dendritic cell. In some embodiments, the modified immune cell is a modified unstimulated immune cell or precursor cell thereof. In some embodiments, the modified immune cell is a modified unstimulated T cell, a modified unstimulated NK cell, or a modified unstimulated NKTcell. In some embodiments, the modified immune cell is a CD4+ T cell or a CD8+ T cell. In some embodiments, the modified immune cell is an allogeneic T cell or autologous human T cell. In some embodiment, the modified immune cell is a human cell, or mammalian cell.

B. Modified Immune Cells

One aspect of the present disclosure provides an isolated modified T cell comprising at least one functionally impaired polypeptide selected from the group consisting of CD3δ, CD3ε, CD3γ, B2M, C2TA, TAP1, TAP2, TAPBP, NLRC5, HLA-DM, RFX5,RFXANK, RFXAP, and invariant chain (Ii Chain). As used herein, the term “functionally impaired polypeptide” means the polypeptide is mutated (e.g. comprises a deletion, an insertion, or is a truncated variant), such that it cannot readily bind to other components of the TCR complex, or that it is not incorporated into the TRC complex. In some embodiments, a functional impaired polypeptide results in a functionally impaired TCR or suppresses the expression of a functional TCR on the cell surface. In some embodiments, the impaired polypeptide may be a dominant negative polypeptide that substantially inhibits the activity of a TCR complex. A modified T cell comprising the recited functionally impaired polypeptide is a TCR-deficient T cell that does not produce a functional TCR, or expresses very little functional TCR at the cell surface. In some embodiments, the impaired polypeptide is a component of the TCR signaling complex. In some embodiments, the impaired polypeptide modulates the formation of a functional TCR. In some embodiments, an impaired polypeptide comprises a mutation that affect the function or expression of a functional protein. In some embodiments, the mutation is a deletion, an insertion, a substitution or a combination thereof. In some embodiments, an impaired polypeptide is caused by the expression of a defective gene product, or absence of expression of a desired gene or gene product.

Proper functioning of the TCR requires the proper stoichiometric ratio of the proteins that compose the TCR complex. As shown in FIG. 1, the TCR complex comprises the variable TCR-alpha chain (TCR-α, TRAC), and TCR-beta chain (TCR-β, TRBC), which are coupled to three dimeric modules CD3δ/CD3ε, CD3γ/CD3ε, and CD3ζ/CD3ζ. The three dimeric modules are an integral part of TCR signaling. Each CD3 receptor comprises signaling motifs that propagate and amplify TCR receptor activation upon engagement of the TCR-α/β heterodimer with a MHC-peptide ligand. Upon ligand binding to TCRαβ, the CD3 subunits undergo conformational changes and the signaling motifs (e.g. ITAMs) within the CD3 cytoplasmic tails become phosphorylated by intracellular protein tyrosine kinases. Subsequently, SH2-domain containing intracellular signaling and adapter molecules are recruited to the plasma membrane, where they amplify the TCR activation signal by directly interacting with the CD3 signaling motifs. Hence, while the TCRαβ heterodimer is responsible for binding of the antigen, the CD3 subunits serve as signal transducing elements. As such, if one of the requisite CD3 receptor is missing or impaired, the TCR complex is functionally destabilized, or at least the signaling function of the TCR is weakened. Without wishing to be bound by theory, the coordinate expression of all six proteins is required for the surface expression of the TCR complex and/or function.

Each component of the TCR complex is required for TCR complex assembly at the cell surface. A loss of one component of the TCR complex can result in loss of TCR expression at the cell surface. In some embodiments, a loss of one component may not abolish surface expression of the TCR complex. In such embodiments, while some or even all TCR expression may remain, it is the TCR receptor function, which determines whether the TCR receptor induces an immune response. The present invention contemplates the functional deficiency, rather than the absence of a complete TCR complex at the cell surface. Without wishing to be bound by theory, the lower the TCR expression, the less likely sufficient TCR cross-linking can occur to lead to T cell activation via the TCR complex.

In some embodiments, the isolated modified T cell comprises two or more functionally impaired polypeptides. In such an embodiments, one impaired polypeptide can be a T-cell receptor α chain (TRAC). The TCR complex will be retained inside the cell, and would not translocate to the plasma membrane in the absence of TRAC. Moreover, the TCR receptor lacking TRAC will be unstable and may be rapidly degraded. In some embodiments, the modified T cell comprises three or more functionally impaired polypeptides selected from TRAC, TRBC, CD3δ, CD3ε, CD3γ, B2M, C2TA, TAP1, TAP2, TAPBP, NLRC5, HLA-DM, RFX5, RFXANK, RFXAP, or the invariant (Ii) chain. In some embodiments, the modified T cell comprises two functionally impaired polypeptides selected from CD3α, CD3δ, CD3ε, CD3γ, B2M, C2TA, TAP1, TAP2, TAPBP, NLRC5, HLA-DM, RFX5, RFXANK, RFXAP, or Ii chain. In some embodiments, the modified T cell comprises three functionally impaired polypeptides selected from CD3α, CD3δ, CD3ε, CD3γ, B2M, C2TA, TAP1, TAP2, TAPBP, NLRC5, HLA-DM, RFX5, RFXANK, RFXAP, or Ii chain. In some embodiments, the modified T cell comprises a functionally impaired polypeptide selected from the group consisting of CD3δ, CD3ε, and CD3γ, and at least one functionally impaired polypeptide selected from TRAC, TRBC, B2M, C2TA, TAP1, TAP2, TAPBP, NLRC5, HLA-DM, RFX5, RFXANK, RFXAP, or Ii Chain. In some embodiments, the modified T cell comprises at least one functionally impaired polypeptide selected from the group consisting of CD3δ, CD3ε, and CD3γ, and at least one a functionally impaired polypeptide selected from TRAC, TRBC, B2M, C2TA, TAP1, TAP2, TAPBP, NLRC5, HLA-DM, RFX5, RFXANK, RFXAP, or Ii Chain.

In some embodiments, the modified T cell comprises (a) at least one of a functionally impaired CD3δ, CD3ε, and/or CD3γ and (b) at least one functionally impaired polypeptide selected from TRAC, TRBC, B2M, C2TA, TAP1, TAP2, TAPBP, NLRC5, HLA-DM, RFX5, RFXANK, RFXAP, or Ii chain. In some embodiments, the modified T cell comprises two or more functionally impaired polypeptides selected from TRAC, TRBC, B2M, C2TA, TAP1, TAP2, TAPBP, NLRC5, HLA-DM, RFX5, RFXANK, RFXAP, or Ii chain.

In some embodiments, the functional impaired polypeptide reduces protein expression. In such an embodiment, the modified T cell has a reduced expression of TRAC, TRBC, CD3δ, CD3ε, CD3γ, B2M, C2TA, TAP1, TAP2, TAPBP, NLRC5, HLA-DM, RFX5, RFXANK, RFXAP, Ii chain, or any combination thereof. In some embodiments, the functional impaired polypeptide is absence of the encoded gene expression. In such an embodiment, the modified T cell does not express CD3δ, CD3ε, CD3γ, TRAC, TRBC, B2M, C2TA, TAP1, TAP2, TAPBP, NLRC5, HLA-DM, RFX5, RFXANK, RFXAP, Ii chain, or any combination thereof.

In some embodiments, the modified T cell comprising reduced expression and or lacking expression of a polypeptide selected from the group consisting of CD3δ, CD3ε, CD3γ, TRAC, TRBC, B2M, C2TA, TAP1, TAP2, TAPBP, NLRC5, HLA-DM, RFX5, RFXANK, RFXAP, Ii chain further comprises a functionally impaired polypeptide selected from TRAC, TRBC, B2M, and C2TA. In such an embodiment, the modified T cell has a reduced expression of TRAC, TRBC, B2M, or C2TA and/or does not express TRAC, TRBC, B2M, or C2TA.

In some embodiments, the modification of CD3δ, CD3ε, and/or CD3γ leads to an impaired TCR/CD3 receptor complex function. In some embodiments, the functionally impaired polypeptide contemplated by the present invention is generated using a gene editing technology. In some embodiments, the gene editing selected from the group consisting of a CRISPR-associated (Cas) (CRISPR-Cas) endonuclease system, a TALEN gene editing system, a zinc finger nuclease (ZFN) gene editing system, a meganuclease gene editing system, or a mega-TALEN gene editing system, antisense RNA, antigomer RNA, RNAi, siRNA, and shRNA. In some embodiments, CD3δ, CD3ε, or CD3γ is modified by targeting one or more exons of CD3δ, CD3ε, or CD3γ, optionally exon 1 of CD3δ, CD3ε, or CD3γ.

Whether a T cell expresses a functional TCR may be determined using known assay methods such as are known in the art, or are described herein. In some embodiments, the expression of TCR αβ and CD3 can be evaluated by flow cytometry and quantitative real-time PCR (QRT-PCR). Expression of TCR-α, TCR-β, CD3ε, CD3δ, CD3γ, and CD3-ζ mRNA can be analyzed by QRT-PCR using an ABI7300 real-time PCR instrument and gene-specific TAQMAN® primers using methods similar to those used in Sentman et al., J. Immunol. 173:6760-6766 (2004). Changes in cell surface expression can be determined using antibodies specific for TCR-α, TCR-β, CD3ε, CD8, CD4, CD5, and CD45. To test for TCR/CD3 expression using Flow cytometry, fluorochrome-labeled antibodies against specific subunits of the TCR complex are used. In some embodiments live cells are stained with, for example, antibodies against CD5, CD8, and CD4, in combination with an antibody against CD3ε, CD3δ, CD3γ, TCRα, TCRβ, TCRγ, or TCRδ. If the expression of either the CD3 or TCR genes is used, the expression of both TCR proteins and CD3 proteins should be severely reduced in the modified T cell when compared to an unmodified T cell, or T cell expressing a control vector. Isotype control antibodies are used to control for background fluorescence.

To determine whether the expression of a functionally impaired polypeptide in the modified T cell is sufficient to alter TCR function and/or the modified T cell function, the modified T cell is tested for: (1) cell survival in vitro; (2) proliferation in the presence of mitomycin C-treated allogeneic PBMCs; and/or (3) cytokine production in response to allogeneic PBMCs, anti-CD3 mAbs, or anti-TCR mAbs.

To test for functional deficiency of the TCR complex, a lack of production of key effector cytokines that drive T cell expansion can be determined. For example, the effect of an anti-CD3 stimulation on modified T cells may be used to determine, the production of Interleukin-2 (IL-2) and/or interferon (IFN)-gamma production.

In some embodiments, the modified T cell that comprises the functionally impaired polypeptide exhibits reduced T cell receptor expression as compared to an unmodified T cell. In some embodiments, the modified T cell that comprises the functionally impaired polypeptide exhibits reduced expression of the impaired polypeptide. In some embodiments, the modified T cell that comprises the functionally impaired polypeptide exhibits complete absence of the T cell receptor complex surface expression. In some embodiments, the modified T cell that comprises the functionally impaired polypeptide exhibits reduced or insufficient T cell receptor cross-linking. In some embodiments, the modified T cell expresses some, or all of TCR subunits may be on the cell surface. Without functional TCRs on their surface, the modified T cell fails to be fully activated. As such, the modified T cell contemplated by the present invention cannot mount an undesirable reaction when introduced into a host. As a result, the modified T cell fail to cause GvHD or HvHD because the modified T cell cannot transduced a signal from the host MHC molecules.

In some embodiments, the modified T cell exerts a reduced immune response in a subject when the modified T cell is administered to the subject, as compared to the immune response exerted by an unmodified T cell administered to the same subject. The modified T cell of the present invention can be used in all application of T cell therapies. In some embodiments, the modified T cell is used in any methods or compositions where T cells therapy is desirable. In some embodiments, the modified T cell of the present invention can be used for reducing or ameliorating, or preventing or treating cancer, GVHD, transplantation rejection, infection, one or more autoimmune disorders, radiation sickness, or other diseases or conditions.

### IV. Gene Editing Systems

A. CRISPR

In some embodiments, the modified immune cell of the present disclosure is a gene edited modified immune cell. In some embodiments, the insertion and/or deletion in one or more gene loci each encoding an endogenous immune protein of the present disclosure is the result of a gene editing. In certain embodiments, the gene encoding CD3δ, CD3ε, CD3γ, TRAC, B2M, CIITA, TAP1, TAP2, TAPBP, NLRC5, HLA-DM, RFX5, RFXANK, RFXAP, or Ii chain is modified by a gene editing system. In some instances, the gene editing system comprises an RNA-guided nuclease such as a clustered regularly interspersed short palindromic nucleic acid (CRISPR)-Cas system. The CRISPR system (also referred to herein as the CRISPR-Cas system, Cas system, or CRISPR/Cas system) comprises a Cas endonuclease and a guide nucleic acid sequence specific for a target gene which after introduction into a cell form a complex that enables the Cas endonuclease to introduce a break (e.g., a double stranded break) at the target gene. In some embodiments, the modified immune cell is edited using CRISPR/Cas9 to disrupt one or more endogenous immune proteins.

In some embodiments, the CRISPR-Cas system is used to disrupt one or more of endogenous CD3δ, CD3ε, CD3γ, TRAC, B2M, CIITA, TAP1, TAP2, TAPBP, NLRC5, HLA-DM, RFX5, RFXANK, RFXAP, or Ii chain, thereby resulting in the downregulation of the gene expression of CD3δ, CD3ε, CD3γ, TRAC, B2M, CIITA, TAP1, TAP2, TAPBP, NLRC5, HLA-DM, RFX5, RFXANK, RFXAP, or Ii chain. In some embodiments, the insertion and/or deletion capable of downregulating gene expression of the one or more endogenous immune proteins downregulate the expression of one ore more endogenous protein selected from the group consisting of CD3δ, CD3ε, CD3γ, TRAC, B2M, CIITA, TAP1, TAP2, TAPBP, NLRC5, HLA-DM, RFX5, RFXANK, RFXAP, or Ii chain. In some embodiments, each of the insertion and/or deletion capable of downregulating gene expression comprises a CRISPR-related system. In some embodiments, the CRISPR-related system is a CRISPR-associated Cas endonuclease and a guide RNA.

In some embodiments, the Cas endonuclease comprises a Cas9 endonuclease. In some instances, the Cas9 endonuclease is derived from or based on, e.g., a Cas9 molecule of S. pyogenes (e.g., SpCas9), S. thermophiles, Staphylococcus aureus (e.g., SaCas9), or Neisseria meningitides. In some instances, the Cas9 endonuclease is derived from or based on, e.g., a Cas9 molecule of Acidovorax avenae, Actinobacillus pleuropneumonias, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium rectale, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

In some embodiments, the Cas9 endonuclease is derived from a Cas9 molecule of: S. pyogenes (e.g., strain SF370, MGAS 10270, MGAS 10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA 159, NN2025), S. macacae (e.g., strain NCTC1 1558), S. gallolylicus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. cmginosus (e.g.; strain F021 1), S. agalactia (e.g., strain NEM316, A909), Listeria monocytogenes (e.g., strain F6854), Listeria innocua (L. innocua, e.g., strain Clip 11262), Enterococcus italicus (e.g., strain DSM 15952), or Enterococcus faecium (e.g., strain 1,23,408).

In some instances, the endonuclease comprises Cas3, Cas4, Cas8a, Cas8b, Cas9, Cas10, Cas10d, Cas12a, Cas12b, Cas12d, Cas12e, Cas12f, Cas12g, Cas12h, Cas12i, Cas13, Cas14, CasX, Cse1, Csy1, Csn2, Cpf1, C2c1, Csm2, Cmr5, Fok1, S. pyogenes Cas9, Staphylococcus aureus Cas9, MAD7 nuclease (a type V CRISPR nuclease), or any combination thereof.

B. Guide RNAs

In some embodiments, the guide nucleic acid is a guide RNA (gRNA) molecule, which directs the Cas-RNA complex to a target sequence. In some instances, the directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In some embodiments, a gRNA molecule consists of a single contiguous polynucleotide molecule, referred to herein as a “single guide RNA” (“sgRNA”). In other embodiments, a gRNA molecule consists of a plurality, usually two, polynucleotide molecules, which are themselves capable of association, usually through hybridization, referred to herein as a “dual guide RNA” (“dgRNA”).

In some cases, the gRNA molecule comprises a crRNA and a tracr, which can be optionally on a single polynucleotide or on separate polynucleotides. In some instances, the crRNA comprises a targeting domain and a region that interacts with a tracr to form a flagpole region. The tracr comprises the portion of the gRNA molecule that binds to a nuclease or other effector molecule. In some embodiments, the tracr comprises a nucleic acid sequence that binds specifically to a Cas endonuclease (e.g., Cas9). In some embodiments, the tracr comprises a nucleic acid sequence that forms part of the flagpole. In some embodiments, the targeting domain is the portion of the gRNA molecule that recognizes, e.g., is complementary to, a protospacer sequence within the target DNA.

A protospacer-adjacent motif (PAM) is a 2-6 base pair DNA sequence located adjacent to the 3′ terminus of the protospacer and recognized by the Cas endonuclease. In some instances, each Cas endonuclease recognizes a specific PAM sequence. Exemplary PAM sequences include NGG sequence recognized by the S. pyogenes Cas9 endonuclease; or NGGNG or NNAGAAW sequence recognized by the S. thermophilus Cas9 endonuclease, where N is any nucleotide. One skilled in the art would understand how to design a gRNA molecule based on the specific Cas endonuclease used along with the PAM sequence in which the Cas endonuclease would recognize.

In some embodiments, the guide RNA comprises a guide sequence that is sufficiently complementary with a target sequence of the endogenous immune protein locus selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain). In some embodiments, the guide RNA comprises a guide sequence that is complementary with a sequence within the one or more gene loci each encoding the immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain). In some embodiments, the guide RNA is complementary with a sequence within one or more exons of CD3δ, CD3ε, or CD3γ. In some embodiments, the guide RNA is complementary with a sequence within exon 1 of CD3δ, CD3ε, or CD3γ. In some embodiments, the gRNA nucleic sequence of CD3δ, CD3ε, CD3γ, B2M, CIITA, TAP1, TAP2, TAPBP, NLRC5, HLA-DM, RFX5, RFXANK, RFXAP, or invariant chain (Ii Chain) has the nucleic acid sequence disclose in Table 3.

In some embodiments, the guide RNA comprises a guide sequence that is complementary with a sequence within the CD3δ gene locus and the guide RNA comprises a nucleic acid sequence set forth in SEQ ID NO: 53. In some embodiments, the guide RNA comprises a guide sequence that is complementary with a sequence within the CD3ε gene locus and the guide RNA comprises a nucleic acid sequence is set forth in SEQ ID NO: 52. In some embodiments, the guide RNA comprises a guide sequence that is complementary with a sequence within the CD3γ gene locus and the guide RNA comprises a nucleic acid sequence set forth in SEQ ID NO: 54. In some embodiments, the guide RNA comprises a guide sequence that is complementary with a sequence within the B2M gene locus and the guide RNA comprises a nucleic acid sequence set forth in SEQ ID NO: 55. In some embodiments, the guide RNA comprises a guide sequence that is complementary with a sequence within the CIITA (C2TA) gene locus and the guide RNA comprises a nucleic acid sequence set forth in SEQ ID NO: 61. In some embodiments, the guide RNA comprises a guide sequence that is complementary with a sequence within the TAP1 gene locus and the guide RNA comprises a nucleic acid sequence set forth in SEQ ID NO: 56. In some embodiments, the guide RNA comprises a guide sequence that is complementary with a sequence within the TAP2 gene locus and the guide RNA comprises a nucleic acid sequence set forth in SEQ ID NO: 57. In some embodiments, the guide RNA comprises a guide sequence that is complementary with a sequence within TAPBP gene locus and the guide RNA comprises a nucleic acid sequence set forth in SEQ ID NO: 58, SEQ ID NO: 59, or any combination thereof. In some embodiments, the guide RNA comprises a guide sequence that is complementary with a sequence within the NLRCS gene locus and the guide RNA comprises a nucleic acid sequence set forth in SEQ ID NO: 60. In some embodiments, the guide RNA comprises a guide sequence that is complementary with a sequence within the HLA-DM gene locus and the guide RNA comprises a nucleic acid sequence set forth in SEQ ID NO: 62. In some embodiments, the guide RNA comprises a guide sequence that is complementary with a sequence within the RFX5 gene locus and the guide RNA comprises a nucleic acid sequence set forth in SEQ ID NO: 63, SEQ ID NO: 64, or a combination thereof. In some embodiments, the guide RNA comprises a guide sequence that is complementary with a sequence within the RFXANK gene locus and the guide RNA comprises a nucleic acid sequence set forth in SEQ ID NO: 65. In some embodiments, the guide RNA comprises a guide sequence that is complementary with a sequence within the RFXAP gene locus and the guide RNA comprises a nucleic acid sequence set forth in SEQ ID NO: 66. In some embodiments, the guide RNA comprises a guide sequence that is complementary with a sequence within the li Ii Chain gene locus and the guide RNA comprises a nucleic acid sequence set forth in SEQ ID NO: 67, SEQ ID NO: 68, or any combination thereof.

In some embodiments, one or more, two or more, three or more, or four or more guide nucleic acids (e.g., guide RNA molecules) are transfected into an immune cell with a Cas endonuclease. In some cases, about one, two, or three guide nucleic acids (e.g., guide RNA molecules) are transfected into an immune cell with a Cas endonuclease. In some cases, about three guide nucleic acids (e.g., guide RNA molecules) are transfected into an immune cell with a Cas endonuclease. In some cases about two guide nucleic acids (e.g., guide RNA molecules) are transfected into an immune cell with a Cas endonuclease. In some cases, about one guide nucleic acid (e.g., guide RNA molecule) is transfected into an immune cell with a Cas endonuclease.

In some embodiment, a vector drives the expression of the CRISPR system. The art is replete with suitable vectors that are useful in the present invention. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present invention may also be used for nucleic acid standard gene delivery protocols. Methods for gene delivery are known in the art. Further, the vector may be provided to a cell in the form of a viral vector. Viral vector technology is well known in the art and is described, for example, in Sambrook et al., 4th Edition, Molecular Cloning: A Laboratory Manual, Cold Spring Harbor Laboratory, New York, 2012; and in other virology and molecular biology manuals. Viruses, which are useful as vectors include, but are not limited to, retroviruses, adenoviruses, adeno-associated viruses, herpes viruses, Sindbis virus, gamma-retrovirus and lentiviruses. Without wishing to be bound by theory, a suitable vector contains an origin of replication functional in at least one organism, a promoter sequence, convenient restriction endonuclease sites, and one or more selectable markers. In some embodiments, the CRISPR/Cas system comprises an expression vector. In some embodiments, the CRISPR/Cas system comprises the pAd5/F35-CRISPR vector.

C. TALEN

In some embodiments, the gene editing system is a TALEN gene editing system. TALENs are produced artificially by fusing a TAL effector DNA binding domain to a DNA cleavage domain. Transcription activator-like effects (TALEs) can be engineered to bind to a target DNA. By combining an engineered TALE with a DNA cleavage domain, a restriction enzyme can be produced which is specific to any target DNA sequence.

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition, and can thus be engineered to bind to a target DNA sequence.

To produce a TALEN, a TALE protein is fused to a nuclease (N) comprising, for example, a wild-type or mutated Fok1 endonuclease. The Fok1 domain functions as a dimer, requiring two constructs with unique DNA binding domains for sites in the target genome with proper orientation and spacing. Specificity and off-target effect can be modulated by changing the number of amino acid residues between the TALE DNA binding domain and the Fok1 cleavage domain and the number of bases between the two individual TALEN binding sites.

D. Zinc Finger Nuclease

In some embodiments, the gene editing system is a zinc finger nuclease (ZFN) gene editing system. The zinc finger nuclease is an artificial nuclease which can be used to modify one or more nucleic acid sites of a target nucleic acid sequence. Similar to the TALEN editing system, a ZFN comprises a Fok1 nuclease domain (or derivative thereof) fused to a DNA-binding domain. In the case of a ZFN, the DNA-binding domain comprises one or more zinc fingers. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences.

The ZFN recognizes non-palindromic DNA sites. To cleave the target site, a pair of ZFNs dimerizes and assembles to opposite strands of the target site. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

E. Meganuclease

In some embodiments, the gene editing system is a meganuclease gene editing system. A meganuclease is an artificial nuclease that recognize 15-40 base-pair cleavage sites. In some instances, meganucleases are grouped into families based on their structural motifs which affect nuclease activity and/or DNA recognition. Members of the LAGLIDADG family are characterized by having either one or two copies of the conserved LAGLIDADG motif. In some instances, the LAGLIDADG meganucleases with a single copy of the LAGLIDADG motif form homodimers, whereas members with two copies of the LAGLIDADG motif are found as monomers. The GIY-YIG family members have a GP-YIG module, which is 70-100 residues long and includes four or five conserved sequence motifs with four invariant residues, two of which are required for activity. The His-Cys box meganucleases are characterized by a highly conserved series of histidines and cysteines over a region encompassing several hundred amino acid residues. The NHN family, the members are defined by motifs containing two pairs of conserved histidines surrounded by asparagine residues. Strategies for engineering a meganuclease with altered DNA-binding specificity (e.g., to bind to a predetermined nucleic acid sequence) are known in the art.

In some instances, the meganuclease is a hybrid nuclease termed megaTAL comprising a TALE domain fused to the N-terminus of a meganuclease. In some cases, the meganuclease is a member of the LAGLIDADG family.

In some embodiments, the gene editing system is a gene silencing system. Exemplary gene silencing system comprises a RNAi-, siRNA-, or shRNA-mediated gene silencing system.

### V. Exogenous Nucleic Acids

The present invention provides modified immune cells or precursor cells thereof comprising an insertion and/or deletion in one or more gene loci encoding an endogenous immune protein that is capable of downregulating the gene expression of the endogenous immune protein and an exogenous nucleic acid. In some embodiments, the exogenous nucleic acid encodes a chimeric antigen receptor (CAR), an engineered T cell receptor (TCR), a Killer cell immunoglobulin-like receptor (KIR), an antigen-binding polypeptide, a cell surface receptor ligand, or a tumor antigen. In some embodiments, disclosed herein is a modified immune cell that expresses an exogenous polypeptide. In some instances, the exogenous nucleic encodes a chimeric antigen receptor (CAR). In some instances, the exogenous nucleic acid encodes an antigen-binding polypeptide. In some instances, the exogenous nucleic acid encodes a Killer cell immunoglobulin-like receptor (KIR). In additional instances, the exogenous nucleic acid encodes a cell surface receptor ligand or a tumor antigen.

A. Chimeric Antigen Receptors

The present invention also includes a modified T cell with downregulated gene expression as described herein and a chimeric antigen receptor (CAR). In some embodiments, the present invention encompasses the modified T cell comprising a CAR or a nucleic acid encoding a CAR, wherein the CAR comprises comprise an antigen-binding domain, a hinge domain, a transmembrane domain, a costimulatory domain, and an intracellular signaling domain. Any modified cell comprising a CAR comprising any antigen binding domain, any hinge, any transmembrane domain, any costimulatory domain, and any intracellular signaling domain described herein is envisioned, and can readily be understood and made by a person of skill in the art in view of the disclosure herein.

The antigen binding domain may be operably linked to another domain of the CAR, such as the transmembrane domain or the intracellular domain, both described herein, for expression in the immune cell. In one embodiment, a first nucleic acid sequence encoding the antigen binding domain is operably linked to a second nucleic acid encoding a transmembrane domain, and further operably linked to a third a nucleic acid sequence encoding an intracellular domain.

The antigen binding domains described herein can be combined with any of the transmembrane domains described herein, any of the intracellular domains or cytoplasmic domains described herein, or any of the other domains described herein that may be included in a CAR of the present invention. A subject CAR of the present invention may also include a spacer domain as described herein. In some embodiments, each of the antigen binding domain, transmembrane domain, and intracellular domain is separated by a linker.

1. Antigen Binding Domain

The antigen binding domain of a CAR is an extracellular region of the CAR for binding to a specific target antigen including proteins, carbohydrates, and glycolipids. In some embodiments, the CAR comprises affinity to a target antigen (e.g. a tumor associated antigen) on a target cell (e.g. a cancer cell). The target antigen may include any type of protein, or epitope thereof, associated with the target cell. For example, the CAR may comprise affinity to a target antigen on a target cell that indicates a particular status of the target cell.

As described herein, a CAR of the present disclosure having affinity for a specific target antigen on a target cell may comprise a target-specific binding domain. In some embodiments, the target-specific binding domain is a murine target-specific binding domain, e.g., the target-specific binding domain is of murine origin. In some embodiments, the target-specific binding domain is a human target-specific binding domain, e.g., the target-specific binding domain is of human origin.

The antigen binding domain can include any domain that binds to the antigen and may include, but is not limited to, a monoclonal antibody, a polyclonal antibody, a synthetic antibody, a human antibody, a humanized antibody, a non-human antibody, and any fragment thereof. Thus, in one embodiment, the antigen binding domain portion comprises a mammalian antibody or a fragment thereof. In some embodiments, the antigen binding domain comprises a full-length antibody. In some embodiments, the antigen binding domain comprises an antigen binding fragment (Fab), e.g., Fab, Fab′, F(ab′)₂, a monospecific Fab₂, a bispecific Fab₂, a trispecific Fab₂, a single-chain variable fragment (scFv), dAb, tandem scFv, VhH, V-NAR, camelid, diabody, minibody, triabody, or tetrabody.

In some embodiments, a CAR of the present disclosure may have affinity for one or more target antigens on one or more target cells. In some embodiments, a CAR may have affinity for one or more target antigens on a single target cell. In such embodiments, the CAR is a bispecific CAR, or a multispecific CAR. In some embodiments, the CAR comprises one or more target-specific binding domains that confer affinity for one or more target antigens. In some embodiments, the CAR comprises one or more target-specific binding domains that confer affinity for the same target antigen. For example, a CAR comprising one or more target-specific binding domains having affinity for the same target antigen could bind distinct epitopes of the target antigen. When a plurality of target-specific binding domains is present in a CAR, the binding domains may be arranged in tandem and may be separated by linker peptides. For example, in a CAR comprising two target-specific binding domains, the binding domains are connected to each other covalently on a single polypeptide chain, through a polypeptide linker, an Fc hinge region, or a membrane hinge region.

As used herein, the term “single-chain variable fragment” or “scFv” is a fusion protein of the variable regions of the heavy (VH) and light chains (VL) of an immunoglobulin (e.g., mouse or human) covalently linked to form a VH:VL heterodimer. The heavy (VH) and light chains (VL) are either joined directly or joined by a peptide-encoding linker or spacer, which connects the N-terminus of the VH with the C-terminus of the VL, or the C-terminus of the VH with the N-terminus of the VL. The terms “linker” and “spacer” are used interchangeably herein. In some embodiments, the antigen binding domain (e.g., Tn-MUC1 binding domain, PSMA binding domain, or mesothelin binding domain) comprises an scFv having the configuration from N-terminus to C-terminus, VH-linker-VL. In some embodiments, the antigen binding domain (e.g., a Tn-MUC1 binding domain, a PSMA binding domain, or a mesothelin binding domain) comprises an scFv having the configuration from N-terminus to C-terminus, VL-linker-VH. Those of skill in the art would be able to select the appropriate configuration for use in the present invention.

The linker is typically rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80(6):1910-1917 (2008) and WO 2014/087010. Various linker sequences are known in the art, including, without limitation, glycine serine (GS) linkers such as (GS)n, (GSGGS)n (SEQ ID NO: 47), (GGGS)n (SEQ ID NO: 48), and (GGGGS)n (SEQ ID NO: 49), where n represents an integer of at least 1. Exemplary linker sequences can comprise amino acid sequences including, without limitation, GGSG (SEQ ID NO: 29), GGSGG (SEQ ID NO: 30), GSGSG (SEQ ID NO: 31), GSGGG (SEQ ID NO: 32), GGGSG (SEQ ID NO: 34), GSSSG (SEQ ID NO: 33), GGGGS (SEQ ID NO: 49), or GGGGSGGGGSGGGGS (SEQ ID NO: 50), and the like. Those of skill in the art would be able to select the appropriate linker sequence for use in the present invention. In one embodiment, an antigen binding domain (e.g., a Tn-MUC1 binding domain, a PSMA binding domain, or a mesothelin binding domain) of the present invention comprises a heavy chain variable region (VH) and a light chain variable region (VL), wherein the VH and VL is separated by the linker sequence having the amino acid sequence GGGGSGGGGSGGGGS (SEQ ID NO: 50). In some embodiments, the linker nucleic acid sequence comprises the nucleotide sequence GGTGGCGGTGGCTCGGGCGGTGGTGGGTCGGGTGGCGGCGGATCT (SEQ ID NO: 51).

Despite removal of the constant regions and the introduction of a linker, scFv proteins retain the specificity of the original immunoglobulin. Single chain Fv polypeptide antibodies can be expressed from a nucleic acid comprising VH- and VL-encoding sequences as described by Huston, et al., Proc. Nat. Acad. Sci. USA 85:5879-5883 (1988). Antagonistic scFvs having inhibitory activity have been described. See e.g., Zhao et al., Hybridoma (Larchmt), 27(6):455-51 (2008). Agonistic scFvs having stimulatory activity have been described. See e.g., Peter et al., J. Biol. Chem., 25278(38):36740-7 (2003).

As used herein, “Fab” refers to a fragment of an antibody structure that binds to an antigen but is monovalent and does not have a Fc portion, for example, an antibody digested by the enzyme papain yields two Fab fragments and an Fc fragment (e.g., a heavy (H) chain constant region; Fc region that does not bind to an antigen).

In some instances, the antigen binding domain may be derived from the same species in which the CAR will ultimately be used. For example, for use in humans, the antigen binding domain of the CAR may comprise a human antibody as described elsewhere herein, or a fragment thereof.

Accordingly, an immune cell, e.g., obtained by a method described herein, can be engineered to express a CAR that target one of the following cancer associated antigens (tumor antigens): CD19; CD20; CD22 (Siglec 2); CD37; CD 123; CD22; CD30; CD 171; CS-1 (also referred to as CD2 subset 1, CRACC, SLAMF7, CD319, and 19A24); C-type lectin-like molecule-1 (CLL-1 or CLECL1); CD33; CD133; epidermal growth factor receptor (EGFR); epidermal growth factor receptor variant III (EGFRvIII); human epidermal growth factor receptor (HER1); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAca-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); Folate receptor alpha; Receptor tyro sine-protein kinase ERBB2 (Her2/neu); Mucin 1, cell surface associated (MUC 1); GalNAca1-O-Ser/Thr (Tn) MUC 1 (TnMUC1); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); tyrosine-protein kinase Met (c-Met); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B 1 (CYP1B 1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES 1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-2 (GPC2); Glypican-3 (GPC3); NKG2D; KRAS; GDNF family receptor alpha-4 (GFRa4); IL13Ra2; Fc receptor-like 5 (FCRL5); and immunoglobulin lambda-like polypeptide 1 (IGLL1).

In some embodiments, the immune cell is engineered to express a CAR that targets CD19, CD20, CD22, BCMA, CD37, Mesothelin, PSMA, PSCA, Tn-MUC1, EGFR, EGFRvIII, c-Met, HER1, HER2, CD33, CD133, GD2, GPC2, GPC3, NKG2D, KRAS, or WT1.

2. Transmembrane Domain

With respect to the transmembrane domain, the CAR can be designed to comprise a transmembrane domain that connects the antigen binding domain of the CAR to the intracellular domain. The transmembrane domain of a subject CAR is a region that is capable of spanning the plasma membrane of a cell (e.g., an immune cell or precursor thereof). The transmembrane domain is for insertion into a cell membrane, e.g., a eukaryotic cell membrane. In some embodiments, the transmembrane domain is interposed between the antigen-binding domain and the intracellular domain of a CAR.

In one embodiment, the transmembrane domain is naturally associated with one or more of the domains in the CAR. In some instances, the transmembrane domain can be selected or modified by amino acid substitution to avoid binding of such domains to the transmembrane domains of the same or different surface membrane proteins to minimize interactions with other members of the receptor complex.

In some embodiments, the transmembrane domain may be derived either from a natural or from a synthetic source. Where the source is natural, the domain may be derived from any membrane-bound or transmembrane protein, e.g., a Type I transmembrane protein. Where the source is synthetic, the transmembrane domain may be any artificial sequence that facilitates insertion of the CAR into a cell membrane, e.g., an artificial hydrophobic sequence. In some embodiments, the transmembrane domain of particular use in this invention includes, without limitation, a transmembrane domain derived from (the alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR). In some embodiments, the transmembrane domain comprises at least a transmembrane region of a protein selected from the group consisting of he alpha, beta or zeta chain of the T-cell receptor, CD28, CD2, CD3 epsilon, CD45, CD4, CD5, CD7, CD8, CD9, CD16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR). In some embodiments, the transmembrane domain may be synthetic. In some embodiments, the synthetic transmembrane domain comprises predominantly hydrophobic residues such as leucine and valine. In certain exemplary embodiments, a triplet of phenylalanine, tryptophan and valine will be found at each end of a synthetic transmembrane domain.

The transmembrane domains described herein can be combined with any of the antigen binding domains described herein, any of the costimulatory signaling domains described herein, any of the intracellular signaling domains described herein, or any of the other domains described herein that may be included in a subject CAR.

In one embodiment, the transmembrane domain comprises a CD8α transmembrane domain. In some embodiments, the transmembrane domain comprises a CD8α transmembrane domain comprising the amino acid sequence set forth in SEQ ID NO: 23. In some embodiments, the transmembrane domain comprises the nucleotide sequence set forth in SEQ ID NO: 24.

In some embodiments, the transmembrane domain comprises a CD28 transmembrane domain. In some embodiments, the CAR comprises a CD28 transmembrane domain comprising the amino acid sequence set forth in SEQ ID NO: 27. In some embodiments, the CD28 transmembrane domain comprises the nucleotide sequence set forth in SEQ ID NO: 28.

Tolerable variations of the transmembrane and/or hinge domain will be known to those of skill in the art, while maintaining its intended function. In some embodiments, the transmembrane domain comprises an amino acid sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the amino acid sequences set forth in SEQ ID NOs: 23 and/or 27. In some embodiments the transmembrane domain is encoded by a nucleic acid sequence comprising the nucleotide sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the nucleotide sequences set forth in SEQ ID NOs: 24 and/or 28. The transmembrane domain may be combined with any hinge domain and/or may comprise one or more transmembrane domains described herein.

In some embodiments, the CAR comprises: any antigen-binding domain, a transmembrane domain selected from the group consisting of the t transmembrane domain of alpha, beta or zeta chain of the T-cell receptor, CD28, CD2, CD3 epsilon, CD45, CD4, CD5, CD7, CD8, CD9, CD 16, CD22, CD33, CD37, CD64, CD80, CD86, CD134 (OX-40), CD137 (4-1BB), CD154 (CD40L), CD278 (ICOS), CD357 (GITR), Toll-like receptor 1 (TLR1), TLR2, TLR3, TLR4, TLR5, TLR6, TLR7, TLR8, TLR9, and a killer immunoglobulin-like receptor (KIR), any costimulatory signaling domains, and any intracellular domains or cytoplasmic domains described herein, or any of the other domains described herein that may be included in the CAR, and optionally a hinge domain.

In some embodiments, the CAR further comprises a spacer domain between the extracellular domain and the transmembrane domain of the CAR, or between the intracellular domain and the transmembrane domain of the CAR. As used herein, the term “spacer domain” generally means any oligo- or polypeptide that functions to link the transmembrane domain to, either the extracellular domain or, the intracellular domain in the polypeptide chain. A spacer domain may comprise up to about 300 amino acids, e.g., about 10 to about 100 amino acids, or about 25 to about 50 amino acids. In some embodiments, the spacer domain may be a short oligo- or polypeptide linker, e.g., between about 2 and about 10 amino acids in length. For example, glycine-serine doublet provides a particularly suitable linker between the transmembrane domain and the intracellular signaling domain of the subject CAR.

Accordingly, the CAR of the present disclosure may comprise any of the transmembrane domains, hinge domains, or spacer domains described herein.

3. Hinge Domain

In some embodiments, the subject CAR of the present invention comprises a hinge region. The hinge region of the CAR is a hydrophilic region which is located between the antigen binding domain and the transmembrane domain. In some embodiments, the hinge domain facilitates proper protein folding for the CAR. In some embodiments, the hinge domain is an optional component for the CAR. In some embodiments, the hinge domain comprises a domain selected from Fc fragments of antibodies, hinge regions of antibodies, CH2 regions of antibodies, CH3 regions of antibodies, artificial hinge sequences or combinations thereof. In some embodiments, the hinge domain is selected from but not limited to, a CD8α hinge, artificial hinges made of polypeptides which may be as small as, three glycines (Gly). In some embodiments, the hinge region is a hinge region polypeptide derived from a receptor. In some embodiments, the hinge region is a CD8-derived hinge region). In one embodiment, the hinge domain comprises an amino acid sequence derived from human CD8, or a variant thereof. In some embodiments, a subject CAR comprises a CD8α hinge domain and a CD8α transmembrane domain. In some embodiment, the CD8α hinge domain comprises the amino acid sequence set forth in SEQ ID NO: 25. In some embodiments, the CD8α hinge domain comprises the nucleotide sequence set forth in SEQ ID NO: 26.

In some embodiments the hinge domain comprises an amino acid sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the amino acid sequences set forth in SEQ ID NO 25. In some embodiments the hinge domain is encoded by a nucleic acid sequence comprising the nucleotide sequence that has at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% sequence identity to any of the nucleotide sequences set forth in SEQ ID NO: 26.

In some embodiments, the hinge domain connects the antigen-binding domain to the transmembrane domain, which, is linked to the intracellular domain. In exemplary embodiments, the hinge region is capable of supporting the antigen binding domain to recognize and bind to the target antigen on the target cells. See e.g., Hudecek et al., Cancer Immunol. Res., 3(2): 125-135 (2015). In some embodiments, the hinge region is a flexible domain, thus allowing the antigen binding domain to have a structure to optimally recognize the specific structure and density of the target antigens on a cell such as tumor cell. The flexibility of the hinge region permits the hinge region to adopt many different conformations.

In some embodiments, the hinge domain has a length selected from about 4 to about 50, from about 4 to about 10, from about 10 to about 15, from about 15 to about 20, from about 20 to about 25, from about 25 to about 30, from about 30 to about 40, or from about 40 to about 50 amino acids.

Suitable hinge regions can be readily selected and can be of any of a number of suitable lengths, such as from about 1 amino acid (e.g., Glycine (Gly) to about 20 amino acids, from about 2 to about 15, from about 3 to about 12 amino acids, including about 4 to about 10, about 5 to about 9, about 6 to about 8, or about 7 to about 8 amino acids, and can be about 1, about 2, about 3, about 4, about 5, about 6, or about 7 amino acids.

In some embodiments, the amino acid is a glycine (Gly). Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see, e.g., Scheraga, Rev. Computational. Chem. (1992) 2: 73-142). In some embodiment, the hinge regions comprises glycine polymers (G)n, glycine-serine polymers. In some embodiments, the hinge region comprises glycine-serine polymers selected from the group consisting of (GS)n, (GSGGS)n (SEQ ID NO: 47) and (GGGS)n (SEQ ID NO: 48), where n is an integer of at least one). In some embodiments, the hinge domain comprises an amino acid sequence of including, but not limited to, GGSG (SEQ ID NO: 29), GGSGG (SEQ ID NO: 30), GSGSG (SEQ ID NO: 31), GSGGG (SEQ ID NO: 32), GGGSG (SEQ ID NO: 34), GSSSG (SEQ ID NO: 33). In some embodiment, the hinge region comprises glycine-alanine polymers, alanine-serine polymers, or other flexible linkers known in the art.

In some embodiments, the hinge region is an immunoglobulin heavy chain hinge region. Immunoglobulin hinge region amino acid sequences are known in the art. In some embodiments, an immunoglobulin hinge domain comprises an amino acid sequence selected from the group consisting of DKTHT (SEQ ID NO: 35); CPPC (SEQ ID NO: 36); CPEPKSCDTPPPCPR (SEQ ID NO: 37) (see, e.g., Glaser et al., J. Biol. Chem. (2005) 280:41494-41503); ELKTPLGDTTHT (SEQ ID NO: 38); KSCDKTHTCP (SEQ ID NO: 39); KCCVDCP (SEQ ID NO: 40); KYGPPCP (SEQ ID NO: 41); EPKSCDKTHTCPPCP (SEQ ID NO: 42) (human IgG1 hinge); ERKCCVECPPCP (SEQ ID NO: 43) (human IgG2 hinge); ELKTPLGDTTHTCPRCP (SEQ ID NO: 44) (human IgG3 hinge); SPNMVPHAHHAQ (SEQ ID NO: 45) (human IgG4 hinge); and the like.

In some embodiments, the hinge region is an immunoglobulin heavy chain hinge region. In some embodiments, the hinge is selected from CH1 and CH3 domains of IgGs (such as human IgG4). In some embodiments, the hinge domain comprises an amino acid sequence of a human IgG1, IgG2, IgG3, or IgG4 hinge domain. In some embodiments, the hinge region can include one or more amino acid substitutions and/or insertions and/or deletions compared to a wild-type (naturally-occurring) hinge region. In some embodiment, histidine at position 229 (His229) of human IgG1 hinge is substituted with tyrosine (Tyr). In some embodiments, the hinge domain comprises the amino acid sequence EPKSCDKTYTCPPCP (SEQ ID NO: 46).

4. Costimulatory Domain

The CAR of the present invention also comprises an intracellular domain. The intracellular domain or otherwise the cytoplasmic domain of the CAR is responsible for activation of the cell in which the CAR is expressed. The term “intracellular domain” is thus meant to include any portion of the intracellular domain sufficient to transduce the activation signal. In one embodiment, the intracellular domain includes a domain responsible for an effector function. The term “effector function” refers to a specialized function of a cell. Effector function of a T cell, for example, may be cytolytic activity or helper activity including the secretion of cytokines. In one embodiment, the intracellular domain of the CAR includes a domain responsible for signal activation and/or transduction. The intracellular domain may transmit signal activation via protein-protein interactions, biochemical changes or other response to alter the cell's metabolism, shape, gene expression, or other cellular response to activation of the chimeric intracellular signaling molecule.

Examples of an intracellular domain for use in the invention include, but are not limited to, the cytoplasmic portion of a T cell receptor (TCR), and any co-stimulatory molecule, or any molecule that acts in concert with the TCR to initiate signal transduction in the T cell, following antigen receptor engagement, as well as any derivative or variant of these elements and any synthetic sequence that has the same functional capability.

In some embodiments, the intracellular domain comprises a costimulatory signaling domain and an intracellular signaling. In certain embodiments, the intracellular domain comprises a costimulatory signaling domain. In one embodiment, the intracellular domain of the CAR comprises a costimulatory signaling domain selected from the group consisting of a portion of a signaling domain from proteins in the TNFR superfamily, CD27, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR, any derivative or variant thereof, any synthetic sequence thereof that has the same functional capability, and any combination thereof.

In some embodiments, the costimulatory domain comprises one or more of a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof. In some embodiments, the costimulatory domain comprises one or more of a costimulatory domain of a protein selected from the group consisting of proteins in the CD28, 4-1BB (CD137), OX40 (CD134), CD27, CD2, or a combination thereof. In some embodiments, the costimulatory signaling domain comprises 4-1BB costimulatory domain. In some embodiments, the costimulatory signaling domain comprises CD2 costimulatory domain. In some embodiments, the costimulatory signaling domain comprises CD28 costimulatory domain.

In one embodiment, the costimulatory domain of the CAR comprises a 4-1BB costimulatory domain comprising the amino acid sequence set forth in SEQ ID NO: 1. In some embodiments, the 4-1BB costimulatory domain is encoded by a nucleic acid sequence comprising the nucleotide sequence set forth in SEQ ID NO: 2 or 3. In some embodiment, the costimulatory domain of the CAR comprises a CD28 costimulatory domain comprising the amino acid sequence set forth in SEQ ID NO: 4. In some embodiments, the CD28 costimulatory domain is encoded by a nucleic acid sequence comprising the nucleotide sequence set forth in SEQ ID NO: 5. In some embodiments, the costimulatory domain of the CAR comprises a CD28(YMFM) costimulatory domain comprising the amino acid sequence set forth in SEQ ID NO: 6. In some embodiments, the CD28(YMFM) costimulatory domain is encoded by a nucleic acid sequence comprising the nucleotide sequence set forth in SEQ ID NO: 7. In one embodiment, the intracellular domain of the CAR comprises an ICOS costimulatory domain comprising the amino acid sequence set forth in SEQ ID NO: 8. In some embodiments, the ICOS costimulatory domain is encoded by a nucleic acid sequence comprising the nucleotide sequence set forth in SEQ ID NO: 9 or SEQ ID NO: 10. In some embodiments, the intracellular domain of the CAR comprises an ICOS(YMNM) costimulatory domain comprising the amino acid sequence set forth in SEQ ID NO: 11. In some embodiments, the ICOS (YIVINM) costimulatory domain is encoded by a nucleic acid sequence comprising the nucleotide sequence set forth in SEQ ID NO: 12. In some embodiments, the intracellular domain of a subject CAR comprises a CD2 costimulatory domain comprising the amino acid sequence set forth in SEQ ID NO: 13. In some embodiments, the CD2 costimulatory domain is encoded by a nucleic acid sequence comprising the nucleotide sequence set forth in SEQ ID NO: 14. In one embodiment, the intracellular domain of the CAR comprises a CD27 costimulatory domain comprising the amino acid sequence set forth in SEQ ID NO: 15. In some embodiments, the CD27 costimulatory domain is encoded by a nucleic acid sequence comprising the nucleotide sequence set forth in SEQ ID NO: 16. In one embodiment, the intracellular domain of the CAR comprises a OX40 costimulatory domain comprising the amino acid sequence set forth in SEQ ID NO: 17. In some embodiments, the OX40 costimulatory domain is encoded by a nucleic acid sequence comprising the nucleotide sequence set forth in SEQ ID NO: 18.

5. Intracellular Domain

In certain embodiments, the intracellular domain comprises an intracellular signaling domain. Examples of the intracellular domain include a fragment or domain from one or more molecules or receptors including, but are not limited to, TCR, CD3 zeta, CD3 gamma, CD3 delta, CD3 epsilon, CD86, common FcR gamma, FcR beta (Fc Epsilon Rib), CD79a, CD79b, Fc gamma R11a, DAP10, DAP12, T cell receptor (TCR), CD2, CD8, CD27, CD28, 4-1BB (CD137), OX9, OX40, CD30, CD40, PD-1, ICOS, a KIR family protein, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD127, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD1Id, ITGAE, CD103, ITGAL, CD11a, LFA-1, ITGAM, CD lib, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD 162), LTBR, LAT, GADS, SLP-76, PAG/Cbp, NKp44, NKp30, NKp46, NKG2D, Toll-like receptor 1 (TLR1), TLR2, TLR3, TLR4, TLR5, TLR6, TLR7, TLR8, TLR9, syk family tyrosine kinases (Syk, ZAP 70, etc.), src family tyrosine kinases (Lck, Fyn, Lyn, etc.), other co-stimulatory molecules described herein, any derivative, variant, or fragment thereof, any synthetic sequence of a co-stimulatory molecule that has the same functional capability, and any combination thereof.

In some embodiments, the intracellular signaling domain comprises an intracellular domain selected from the group consisting of cytoplasmic signaling domains of a human CD2, CD3 zeta chain (CD3ζ), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof. In some embodiments, the intracellular signaling domain comprises CD3 zeta intracellular signaling domain.

Additional examples of intracellular domains include, without limitation, intracellular signaling domains of several types of various other immune signaling receptors, including, but not limited to, first, second, and third generation T cell signaling proteins including CD3, B7 family costimulatory, and Tumor Necrosis Factor Receptor (TNFR) superfamily receptors. Additionally, intracellular signaling domains may include signaling domains used by NK and NKT cells such as signaling domains of NKp30 (B7-H6), and DAP 12, NKG2D, NKp44, NKp46, DAP10, and CD3z.

Intracellular signaling domains suitable for use in the CAR of the present invention include any desired signaling domain that transduces a signal in response to the activation of the CAR (i.e., activated by antigen and dimerizing agent). In some embodiments, a distinct and detectable signal e.g. comprises increased production of one or more cytokines by the cell; change in transcription of a target gene; change in activity of a protein; change in cell behavior (e.g., cell death); cellular proliferation; cellular differentiation; cell survival; and/or modulation of cellular signaling responses. e.g. In some embodiments, the intracellular signaling domain includes DAP10/CD28 type signaling chains. In some embodiments, the intracellular signaling domain is not covalently attached to the membrane bound CAR, but is instead diffused in the cytoplasm.

Intracellular signaling domains suitable for use in the CAR of the present invention include immunoreceptor tyrosine-based activation motif (ITAM)-containing intracellular signaling polypeptides. In some embodiments, the intracellular signaling domain includes at least one at least two, at least three, at least four, at least five, or at least six ITAM motifs as described below. In some embodiments, an ITAM motif is repeated twice in an intracellular signaling domain, where the first and second instances of the ITAM motif are separated from one another by 6 to 8 amino acids. In one embodiment, the intracellular signaling domain of a subject CAR comprises 3 ITAM motifs. In some embodiments, intracellular signaling domains includes the signaling domains of human immunoglobulin receptors that contain immunoreceptor tyrosine based activation motifs (ITAMs) such as, but not limited to, Fc gamma RI, Fc gamma RIIA, Fc gamma RIIC, Fc gamma RIIIA, FcRL5.

A suitable intracellular signaling domain can be an ITAM motif-containing portion that is derived from a polypeptide that contains an ITAM motif. For example, a suitable intracellular signaling domain can be an ITAM motif-containing domain from any ITAM motif-containing protein. Thus, a suitable intracellular signaling domain need not contain the entire sequence of the entire protein from which it is derived. Examples of suitable ITAM motif-containing polypeptides include, but are not limited to: DAP12, FCER1G (Fc epsilon receptor I gamma chain), CD3D (CD3 delta), CD3E (CD3 epsilon), CD3G (CD3 gamma), CD3Z (CD3 zeta), and CD79A (antigen receptor complex-associated protein alpha chain).

In one embodiment, the intracellular signaling domain is derived from DAP12 (also known as TYROBP; TYRO protein tyrosine kinase binding protein; KARAP; PLOSL; DNAX-activation protein 12; KAR-associated protein; TYRO protein tyrosine kinase-binding protein; killer activating receptor associated protein; killer-activating receptor-associated protein; etc.). In one embodiment, the intracellular signaling domain is derived from FCER1G (also known as FCRG; Fc epsilon receptor I gamma chain; Fc receptor gamma-chain; fc-epsilon RI-gamma; fcR gamma; fceR1 gamma; high affinity immunoglobulin epsilon receptor subunit gamma; immunoglobulin E receptor, high affinity, gamma chain; etc.). In one embodiment, the intracellular signaling domain is derived from T-cell surface glycoprotein CD3 delta chain (also known as CD3D; CD3-DELTA; T3D; CD3 antigen, delta subunit; CD3 delta; CD3d antigen, delta polypeptide (TiT3 complex); OKT3, delta chain; T-cell receptor T3 delta chain; T-cell surface glycoprotein CD3 delta chain; etc.). In one embodiment, the intracellular signaling domain is derived from T-cell surface glycoprotein CD3 epsilon chain (also known as CD3e, T-cell surface antigen T3/Leu-4 epsilon chain, T-cell surface glycoprotein CD3 epsilon chain, AI504783, CD3, CD3epsilon, T3e, etc.). In one embodiment, the intracellular signaling domain is derived from T-cell surface glycoprotein CD3 gamma chain (also known as CD3G, T-cell receptor T3 gamma chain, CD3-GAMMA, T3G, gamma polypeptide (TiT3 complex), etc.). In one embodiment, the intracellular signaling domain is derived from T-cell surface glycoprotein CD3 zeta chain (also known as CD3Z, T-cell receptor T3 zeta chain, CD247, CD3-zeta, CD3H, CD3Q, T3Z, TCRZ, etc.). In one embodiment, the intracellular signaling domain is derived from CD79A (also known as B-cell antigen receptor complex-associated protein alpha chain; CD79a antigen (immunoglobulin-associated alpha); MB-1 membrane glycoprotein; Ig-alpha; membrane-bound immunoglobulin-associated protein; surface IgM-associated protein; etc.). In one embodiment, an intracellular signaling domain suitable for use in the CAR of the present disclosure includes a DAP10/CD28 type signaling chain. In one embodiment, an intracellular signaling domain suitable for use in a subject CAR of the present disclosure includes a ZAP70 polypeptide. In some embodiments, the intracellular signaling domain includes a cytoplasmic signaling domain of TCR zeta, FcR gamma, FcR beta, CD3 gamma, CD3 delta, CD3 epsilon, CD5, CD22, CD79a, CD79b, or CD66d. In one embodiment, the intracellular signaling domain in the CAR includes a cytoplasmic signaling domain of human CD3 zeta.

While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The intracellular signaling domain includes any truncated portion of the intracellular signaling domain sufficient to transduce the effector function signal.

The intracellular signaling domains described herein can be combined with any of the costimulatory signaling domains described herein, any of the antigen binding domains described herein, any of the transmembrane domains described herein, or any of the other domains described herein that may be included in the CAR. In some embodiment, the intracellular domain of the CAR comprises dual signaling domains. The dual signaling domains may include a fragment or domain from any of the molecules described herein. In some embodiments, the intracellular domain comprises 4-1BBcostimulatory domain and CD3 zeta signaling domain; CD28 costimulatory domain and CD3 zeta signaling domain; CD2 costimulatory domain and CD3 zeta signaling domain. In some embodiments, the intracellular domain of the CAR includes any portion of a co-stimulatory molecule, such as at least one signaling domain from CD3, CD27, CD28, ICOS, 4-1BB, PD-1, T cell receptor (TCR), any derivative or variant thereof, any synthetic sequence thereof that has the same functional capability, and any combination thereof.

Further, variant intracellular signaling domains suitable for use in a subject CAR are known in the art. The YMFM motif is found in ICOS and is a SH2 binding motif that recruits both p85 and p50alpha subunits of PI3K, resulting in enhanced AKT signaling. In one embodiment, a CD28 intracellular domain variant may be generated to comprise a YMFM motif.

In one embodiment, the intracellular domain of a subject CAR comprises a CD3 zeta intracellular signaling domain comprising the amino acid sequence set forth in SEQ ID NO: 19 or SEQ ID NO: 21, which may be encoded by a nucleic acid sequence comprising the nucleotide sequence set forth in SEQ ID NO: 20 or SEQ ID NO: 22, respectively.

Tolerable variations of the intracellular domain will be known to those of skill in the art, while maintaining specific activity. In some embodiments, the intracellular domain comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the amino acid sequences set forth in SEQ ID NO: 19 or 21. In some embodiments, the intracellular domain is encoded by a nucleic acid sequence comprising a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to any of the nucleotide sequences set forth in SEQ ID NO: 20 or 22.

In one embodiment, the intracellular domain of a subject CAR comprises an ICOS costimulatory domain and a CD3 zeta intracellular signaling domain. In one embodiment, the intracellular domain of a subject CAR comprises a CD28 costimulatory domain and a CD3 zeta intracellular signaling domain. In one embodiment, the intracellular domain of a subject CAR comprises a CD28 YMFM variant costimulatory domain and a CD3 zeta intracellular signaling domain. In one embodiment, the intracellular domain of a subject CAR comprises a CD27 costimulatory domain and a CD3 zeta intracellular signaling domain. In one embodiment, the intracellular domain of a subject CAR comprises a OX40 costimulatory domain and a CD3 zeta intracellular signaling domain. In one exemplary embodiment, the intracellular domain of a subject CAR comprises a 4-1BB costimulatory domain and a CD3 zeta intracellular signaling domain. In one exemplary embodiment, the intracellular domain of a subject CAR comprises a CD2 costimulatory domain and a CD3 zeta intracellular signaling domain.

Table 1 illustrates exemplary sequences of the domains of a CAR described herein.

TABLE 1

| SEQ ID | | |
|---|---|---|
| NO: | Description | Sequence |
| 1 | 4-1BB costimulatory | KRGRKKLLYIFKQPFMRPVQTTQEEDGCSCRFPEEEEGGCE |
| | domain amino acid | L |
| 2 | 4-1BB costimulatory | AAACGGGGCAGAAAGAAACTCCTGTATATATTCAAACAACC |
| | domain nucleic acid | ATTTATGAGACCAGTACAAACTACTCAAGAGGAAGACGGCT |
| | sequence #1 | GTAGCTGCCGATTTCCAGAAGAAGAAGAAGGAGGATGTGAA |
| | | CTG |
| 3 | 4-1BB costimulatory | AAACGGGGCAGAAAGAAACTCCTGTATATATTCAAACAACC |
| | domain nucleic acid | ATTTATGAGACCAGTACAAACTACTCAAGAGGAAGATGGCT |
| | sequence #2 | GTAGCTGCCGATTTCCAGAAGAAGAAGAAGGAGGATGTGAA |
| | | CTG |
| 4 | CD28 costimulatory | RSKRSRLLHSDYMNMTPRRPGPTRKHYQPYAPPRDFAAYRS |
| | domain amino acid | |
| | sequence | |
| 5 | CD28 costimulatory | AGGAGTAAGAGGAGCAGGCTCCTGCACAGTGACTACATGAA |
| | domain nucleic acid | CATGACTCCCCGCCGCCCCGGGCCCACCCGCAAGCATTACC |
| | sequence | AGCCCTATGCCCCACCACGCGACTTCGCAGCCTATCGCTCC |
| 6 | CD28(YMFM) | RSKRSRLLHSDYMFMTPRRPGPTRKHYQPYAPPRDFAAYRS |
| | costimulatory domain | |
| | amino acid sequence | |
| 7 | CD28(YMFM) | AGGAGTAAGAGGAGCAGGCTCCTGCACAGTGACTACATGTT |
| | costimulatory domain | CATGACTCCCCGCCGCCCCGGGCCCACCCGCAAGCATTACC |
| | nucleic acid sequence | AGCCCTATGCCCCACCACGCGACTTCGCAGCCTATCGCTCC |
| 8 | ICOS costimulatory | TKKKYSSSVHDPNGEYMFMRAVNTAKKSRLTDVTL |
| | domain amino acid | |
| | sequence | |
| 9 | ICOS costimulatory | ACAAAAAAGAAGTATTCATCCAGTGTGCACGACCCTAACGG |
| | domain nucleic acid | TGAATACATGTTCATGAGAGCAGTGAACACAGCCAAAAAAT |
| | sequence #1 | CCAGACTCACAGATGTGACCCTA |
| 10 | ICOS costimulatory | ACAAAAAAGAAGTATTCATCCAGTGTGCACGACCCTAACGG |
| | domain nucleic acid | TGAATACATGTTCATGAGAGCAGTGAACACAGCCAAAAAAT |
| | sequence #2 | CTAGACTCACAGATGTGACCCTA |
| 11 | ICOS(YMNM) | TKKKYSSSVHDPNGEYMNMRAVNTAKKSRLTDVTL |
| | costimulatory domain | |
| | amino acid sequence | |
| 12 | ICOS(YMNM) | ACAAAAAAGAAGTATTCATCCAGTGTGCACGACCCTAACGG |
| | costimulatory domain | TGAATACATGAACATGAGAGCAGTGAACACAGCCAAAAAAT |
| | nucleic acid sequence | CCAGACTCACAGATGTGACCCTA |
| 13 | CD2 costimulatory | TKRKKQRSRRNDEELETRAHRVATEERGRKPHQIPASTPQN |
| | domain amino acid | PATSQHPPPPPGHRSQAPSHRPPPPGHRVQHQPQKRPPAPS |
| | sequence | GTQVHQQKGPPLPRPRVQPKPPHGAAENSLSPSSN |
| 14 | CD2 costimulatory | ACCAAAAGGAAAAAACAGAGGAGTCGGAGAAATGATGAGGA |
| | domain nucleic acid | GCTGGAGACAAGAGCCCACAGAGTAGCTACTGAAGAAAGGG |
| | sequence | GCCGGAAGCCCCACCAAATTCCAGCTTCAACCCCTCAGAAT |
| | | CCAGCAACTTCCCAACATCCTCCTCCACCACCTGGTCATCG |
| | | TTCCCAGGCACCTAGTCATCGTCCCCCGCCTCCTGGACACC |
| | | GTGTTCAGCACCAGCCTCAGAAGAGGCCTCCTGCTCCGTCG |
| | | GGCACACAAGTTCACCAGCAGAAAGGCCCGCCCCTCCCCAG |
| | | ACCTCGAGTTCAGCCAAAACCTCCCCATGGGGCAGCAGAAA |
| | | ACTCATTGTCCCCTTCCTCTAAT |
| 15 | CD27 costimulatory | QRRKYRSNKGESPVEPAEPCRYSCPREEEGSTIPIQEDYRK |
| | domain amino acid | PEPACSP |
| 16 | CD27 costimulatory | CAACGAAGGAAATATAGATCAAACAAAGGAGAAAGTCCTGT |
| | domain nucleic acid | GGAGCCTGCAGAGCCTTGTCGTTACAGCTGCCCCAGGGAGG |
| | sequence | AGGAGGGCAGCACCATCCCCATCCAGGAGGATTACCGAAAA |
| | | CCGGAGCCTGCCTGCTCCCCC |
| 17 | OX40 costimulatory | ALYLLRRDQRLPPDAHKPPGGGSFRTPIQEEQADAHSTLAK |
| | domain amino acid | I |
| | sequence | |
| 18 | OX40 costimulatory | GCCCTGTACCTGCTCCGCAGGGACCAGAGGCTGCCCCCCGA |
| | domain nucleic acid | TGCCCACAAGCCCCCTGGGGGAGGCAGTTTCAGGACCCCCA |
| | sequence | TCCAAGAGGAGCAGGCCGACGCCCACTCCACCCTGGCCAAG |
| | | ATC |
| 19 | CD3 zeta intracellular | RVKFSRSADAPAYQQGQNQLYNELNLGRREEYDVLDKRRGR |
| | signaling domain amino | DPEMGGKPRRKNPQEGLYNELQKDKMAEAYSEIGMKGERRR |
| | acid sequence | GKGHDGLYQGLSTATKDTYDALHMQALPPR |
| 20 | CD3 zeta intracellular | AGAGTGAAGTTCAGCAGGAGCGCAGACGCCCCCGCGTACCA |
| | signaling domain | GCAGGGCCAGAACCAGCTCTATAACGAGCTCAATCTAGGAC |
| | nucleic acid sequence | GAAGAGAGGAGTACGATGTTTTGGACAAGAGACGTGGCCGG |
| | | GACCCTGAGATGGGGGGAAAGCCGAGAAGGAAGAACCCTCA |
| | | GGAAGGCCTGTACAATGAACTGCAGAAAGATAAGATGGCGG |
| | | AGGCCTACAGTGAGATTGGGATGAAAGGCGAGCGCCGGAGG |
| | | GGCAAGGGGCACGATGGCCTTTACCAGGGTCTCAGTACAGC |
| | | CACCAAGGACACCTACGACGCCCTTCACATGCAGGCCCTGC |
| | | CCCCTCGC |
| 21 | CD3 zeta (Q14K) | RVKFSRSADAPAYKQGQNQLYNELNLGRREEYDVLDKRRGR |
| | intracellular | DPEMGGKPRRKNPQEGLYNELQKDKMAEAYSEIGMKGERRR |
| | signaling domain amino | GKGHDGLYQGLSTATKDTYDALHMQALPPR |
| | acid sequence | |
| 22 | CD3 zeta (Q14K) | AGAGTGAAGTTCAGCAGGAGCGCAGACGCCCCCGCGTACAA |
| | intracellular | GCAGGGCCAGAACCAGCTCTATAACGAGCTCAATCTAGGAC |
| | signaling domain | GAAGAGAGGAGTACGATGTTTTGGACAAGAGACGTGGCCGG |
| | nucleic acid sequence | GACCCTGAGATGGGGGGAAAGCCGAGAAGGAAGAACCCTCA |
| | | GGAAGGCCTGTACAATGAACTGCAGAAAGATAAGATGGCGG |
| | | AGGCCTACAGTGAGATTGGGATGAAAGGCGAGCGCCGGAGG |
| | | GGCAAGGGGCACGATGGCCTTTACCAGGGTCTCAGTACAGC |
| | | CACCAAGGACACCTACGACGCCCTTCACATGCAGGCCCTGC |
| | | CCCCTCGC |
| 23 | CD8 alpha (CD8α) | IYIWAPLAGTCGVLLLSLVITLYC |
| | transmembrane domain | |
| | amino acid sequence | |
| 24 | CD8 alpha | ATCTACATCTGGGCGCCCTTGGCCGGGACTTGTGGGGTCCT |
| | (CD8α)transmembrane | TCTCCTGTCACTGGTTATCACCCTTTACTGC |
| | domain nucleic acid | |
| | sequence | |
| 25 | CD8 alpha (CD8α) hinge | TTTPAPRPPTPAPTIASQPLSLRPEACRPAAGGAVHTRGLD |
| | domain amino acid | FACD |
| | sequence | |
| 26 | CD8 alpha (CD8α) hinge | ACCACGACGCCAGCGCCGCGACCACCAACACCGGCGCCCAC |
| | domain nucleic acid | CATCGCGTCGCAGCCCCTGTCCCTGCGCCCAGAGGCGTGCC |
| | sequence | GGCCAGCGGCGGGGGGCGCAGTGCACACGAGGGGGCTGGAC |
| | | TTCGCCTGTGAT |
| 27 | CD28 transmembrane | FWVLVVVGGVLACYSLLVTVAFIIFWV |
| | domain amino acid | |
| | sequence | |
| 28 | CD28 transmembrane | TTTTGGGTGCTGGTGGTGGTTGGTGGAGTCCTGGCTTGCTA |
| | domain nucleic acid | TAGCTTGCTAGTAACAGTGGCCTTTATTATTTTCTGGGTG |
| | sequence | |
| 29 | Hinge/linker | GGSG |
| 30 | Hinge/linker | GGSGG |
| 31 | Hinge/linker | GSGSG |
| 32 | Hinge/linker | GSGGG |
| 33 | Hinge/linker | GSSSG |
| 34 | Hinge/linker | GGGSG |
| 35 | Ig hinge region | DKTHT |
| 36 | Ig hinge region | CPPC |
| 37 | Ig hinge region | CPEPKSCDTPPPCPR |
| 38 | Ig hinge region | ELKTPLGDTTHT |
| 39 | Ig hinge region | KSCDKTHTCP |
| 40 | Ig hinge region | KCCVDCP |
| 41 | Ig hinge region | KYGPPCP |
| 42 | human IgG1 hinge | EPKSCDKTHTCPPCP |
| 43 | human IgG2 hinge | ERKCCVECPPCP |
| 44 | human IgG3 hinge | ELKTPLGDTTHTCPRCP |
| 45 | human IgG4 hinge | SPNMVPHAHHAQ |
| 46 | human IgG1^{H229Y} hinge | EPKSCDKTYTCPPCP |
| 47 | Hinge/linker | (GSGGS)n |
| 48 | Hinge/linker | (GGGS)n |
| 49 | Hinge/linker | (GGGGS)n |
| 50 | Hinge/linker | GGGGSGGGGSGGGGS |
| 51 | Hinge/linker | GGTGGCGGTGGCTCGGGCGGTGGTGGGTCGGGTGGCGGCGG |
| | | ATCT |
| 52 | CD3epsilon | AGATCCAGGATACTGAGGGCA |
| 53 | CD3delta | TCTCTGGCCTGGTACTGGCTA |
| 54 | CD3gamma | GCTTCTGCATCACAAGTCAGA |
| 55 | B2M | TATCTCTTGTACTACACTGA |
| 56 | TAP1 | GCTCTTGGAGCCAACCGTTG |
| 57 | TAP2 | CTTCCTCAAGGGCTGCCAGGA |
| 58 | TAPBP_gRNA1 | CCTACATGCCCCCCACCTCC |
| 59 | TAPBP_gRNA2 | CGCTCGCATCCTCCACGAAC |
| 60 | NLRC5 | GTGAGCAGCCTCACAAGACAG |
| 61 | C2TA | CCTTGGGGCTCTGACAGGTA |
| 62 | HLA-DMA | CCAGAACACTCGGGTGCCTCG |
| 63 | RFX5_gRNA1 | CAAGGCCGTGCAGAACAAAGT |
| 64 | RFX5_gRNA2 | TTCTGCACGGCCTTGGAAATG |
| 65 | RFXANK | CCTGCACCCCTGAGCCTGTGA |
| 66 | RFXAP | GAGGATCTAGAGGACGAGGAG |
| 67 | Ii Chain_gRNA1 | CATCCTGGTGACTCTGCTCCT |
| 68 | Ii Chain_gRNA2 | TCCAGCCGGCCCTGCTGCTGG |
| 69 | Tn-MUC1 CAR | ATGGCCTTACCAGTGACCGCCTTGCTCCTGCCGCTGGCCTT |
| | nucleic acid sequence | GCTGCTCCACGCCGCCAGGCCGGGATCCCAGGTGCAGCTGC |
| | | AGCAGTCTGATGCCGAGCTCGTGAAGCCTGGCAGCAGCGTG |
| | | AAGATCAGCTGCAAGGCCAGCGGCTACACCTTCACCGACCA |
| | | CGCCATCCACTGGGTCAAGCAGAAGCCTGAGCAGGGCCTGG |
| | | AGTGGATCGGCCACTTCAGCCCCGGCAACACCGACATCAAG |
| | | TACAACGACAAGTTCAAGGGCAAGGCCACCCTGACCGTGGA |
| | | CAGAAGCAGCAGCACCGCCTACATGCAGCTGAACAGCCTGA |
| | | CCAGCGAGGACAGCGCCGTGTACTTCTGCAAGACCAGCACC |
| | | TTCTTTTTCGACTACTGGGGCCAGGGCACAACCCTGACAGT |
| | | GTCTAGCGGAGGCGGAGGATCTGGCGGCGGAGGAAGTGGCG |
| | | GAGGGGGATCTGAACTCGTGATGACCCAGAGCCCCAGCTCT |
| | | CTGACAGTGACAGCCGGCGAGAAAGTGACCATGATCTGCAA |
| | | GTCCTCCCAGAGCCTGCTGAACTCCGGCGACCAGAAGAACT |
| | | ACCTGACCTGGTATCAGCAGAAACCCGGCCAGCCCCCCAAG |
| | | CTGCTGATCTTTTGGGCCAGCACCCGGGAAAGCGGCGTGCC |
| | | CGATAGATTCACAGGCAGCGGCTCCGGCACCGACTTTACCC |
| | | TGACCATCAGCTCCGTGCAGGCCGAGGACCTGGCCGTGTAT |
| | | TACTGCCAGAACGACTACAGCTACCCCCTGACCTTCGGAGC |
| | | CGGCACCAAGCTGGAACTGAAGTCCGGAACCACGACGCCAG |
| | | CGCCGCGACCACCAACACCGGCGCCCACCATCGCGTCGCAG |
| | | CCCCTGTCCCTGCGCCCAGAGGCGTGCCGGCCAGCGGCGGG |
| | | GGGCGCAGTGCACACGAGGGGGCTGGACTTCGCCTGTGATA |
| | | TCTACATCTGGGCGCCCTTGGCCGGGACTTGTGGGGTCCTT |
| | | CTCCTGTCACTGGTTATCACCCTTTACTGCACCAAAAGGAA |
| | | AAAACAGAGGAGTCGGAGAAATGATGAGGAGCTGGAGACAA |
| | | GAGCCCACAGAGTAGCTACTGAAGAAAGGGGCCGGAAGCCC |
| | | CACCAAATTCCAGCTTCAACCCCTCAGAATCCAGCAACTTC |
| | | CCAACATCCTCCTCCACCACCTGGTCATCGTTCCCAGGCAC |
| | | CTAGTCATCGTCCCCCGCCTCCTGGACACCGTGTTCAGCAC |
| | | CAGCCTCAGAAGAGGCCTCCTGCTCCGTCGGGCACACAAGT |
| | | TCACCAGCAGAAAGGCCCGCCCCTCCCCAGACCTCGAGTTC |
| | | AGCCAAAACCTCCCCATGGGGCAGCAGAAAACTCATTGTCC |
| | | CCTTCCTCTAATATCGATAGAGTGAAGTTCAGCAGGAGCGC |
| | | AGACGCCCCCGCGTACAAGCAGGGCCAGAACCAGCTCTATA |
| | | ACGAGCTCAATCTAGGACGAAGAGAGGAGTACGATGTTTTG |
| | | GACAAGAGACGTGGCCGGGACCCTGAGATGGGGGGAAAGCC |
| | | GAGAAGGAAGAACCCTCAGGAAGGCCTGTACAATGAACTGC |
| | | AGAAAGATAAGATGGCGGAGGCCTACAGTGAGATTGGGATG |
| | | AAAGGCGAGCGCCGGAGGGGCAAGGGGCACGATGGCCTTTA |
| | | CCAGGGTCTCAGTACAGCCACCAAGGACACCTACGACGCCC |
| | | TTCACATGCAGGCCCTGCCCCCTCGC |
| 70 | Tn-MUC1 CAR | MALPVTALLLPLALLLHAARPGSQVQLQQSDAELVKPGSSV |
| | amino acid sequence | KISCKASGYTFTDHAIHWVKQKPEQGLEWIGHFSPGNTDIK |
| | | YNDKFKGKATLTVDRSSSTAYMQLNSLTSEDSAVYFCKTST |
| | | FFFDYWGQGTTLTVSSGGGGSGGGGSGGGGSELVMTOSPSS |
| | | LTVTAGEKVTMICKSSQSLLNSGDQKNYLTWYOQKPGQPPK |
| | | LLIFWASTRESGVPDRFTGSGSGTDFTLTISSVQAEDLAVY |
| | | YCONDYSYPLTFGAGTKLELKSGTTTPAPRPPTPAPTIASQ |
| | | PLSLRPEACRPAAGGAVHTRGLDFACDIYIWAPLAGTCGVL |
| | | LLSLVITLYCTKRKKQRSRRNDEELETRAHRVATEERGRKP |
| | | HQIPASTPONPATSQHPPPPPGHRSQAPSHRPPPPGHRVQH |
| | | QPQKRPPAPSGTQVHQQKGPPLPRPRVQPKPPHGAAENSLS |
| | | PSSNIDRVKFSRSADAPAYKQGQNQLYNELNLGRREEYDVL |
| | | DKRRGRDPEMGGKPRRKNPQEGLYNELQKDKMAEAYSEIGM |
| | | KGERRRGKGHDGLYQGLSTATKDTYDALHMQALPPR |
| 71 | Mesothelin CAR | MALPVTALLLPLALLLHAARPQVQLVQSGAEVEKPGASVKV |
| | amino acid sequence | SCKASGYIFIDYYMHWVRQAPGQGLEWMGWINPNSGGTNYA |
| | (M5) | QKFQGRVTMTRDTSISTAYMELSRLRSDDTAVYYCASGWDF |
| | | DYWGQGTLVTVSSGGGGSGGGGSGGGGSGGGGSDIVMTQSP |
| | | SSLSASVGDRVTITCRASQSIRYYLSWYQQKPGKAPKLLIY |
| | | TASILQNGVPSRFSGSGSGTDFTLTISSLQPEDFATYYCLQ |
| | | TYTTPDFGPGTKVEIKTTTPAPRPPTPAPTIASQPLSLRPE |
| | | ACRPAAGGAVHTRGLDFACDIYIWAPLAGTCGVLLLSLVIT |
| | | LYCKRGRKKLLYIFKQPFMRPVQTTQEEDGCSCRFPEEEEG |
| | | GCELRVKFSRSADAPAYKQGQNQLYNELNLGRREEYDVLDK |
| | | RRGRDPEMGGKPRRKNPQEGLYNELQKDKMAEAYSEIGMKG |
| | | ERRRGKGHDGLYQGLSTATKDTYDALHMQALPPR |
| 72 | Mesothelin CAR | MALPVTALLLPLALLLHAARPQVQLQQSGAEVKKPGASVKV |
| | amino acid sequence | SCKASGYTFTGYYMHWVRQAPGQGLEWMGWINPNSGGTNYA |
| | (M11) | QNFQGRVTMTRDTSISTAYMELRRLRSDDTAVYYCASGWDF |
| | | DYWGQGTLVTVSSGGGGSGGGGSGGGGSGGGGSDIRMTQSP |
| | | SSLSASVGDRVTITCRASQSIRYYLSWYQQKPGKAPKLLIY |
| | | TASILQNGVPSRFSGSGSGTDFTLTISSLQPEDFATYYCLQ |
| | | TYTTPDFGPGTKVEIKTTTPAPRPPTPAPTIASQPLSLRPE |
| | | ACRPAAGGAVHTRGLDFACDIYIWAPLAGTCGVLLLSLVIT |
| | | LYCKRGRKKLLYIFKQPFMRPVQTTQEEDGCSCRFPEEEEG |
| | | GCELRVKFSRSADAPAYKQGQNQLYNELNLGRREEYDVLDK |
| | | RRGRDPEMGGKPRRKNPQEGLYNELQKDKMAEAYSEIGMKG |
| | | ERRRGKGHDGLYQGLSTATKDTYDALHMQALPPR |
| 73 | Humanized PSMA- | EVQLVQSGAEVKKPGASVKVSCKASGYTFTEYTIHWVRQAP |
| | specific binding domain | GKGLEWIGNINPNNGGTTYNQKFEDRVTITVDKSTSTAYME |
| | amino acid sequence | LSSLRSEDTAVYYCAAGWNFDYWGQGTTVTVSSGGGGSGGG |
| | | GSSGGGSDIQMTQSPSTLSASVGDRVTITCKASQDVGTAVD |
| | | WYQQKPGQAPKLLIYWASTRHTGVPDRFSGSGSGTDFTLTI |
| | | SRLQPEDFAVYYCQQYNSYPLTFGQGTKVDIK |
| 74 | Humanized PSMA- | GAGGTCCAGCTGGTGCAGTCTGGAGCTGAGGTGAAGAAGCC |
| | specific binding domain | TGGGGCCTCAGTGAAGGTCTCCTGCAAGGCTTCTGGATACA |
| | nucleic acid sequence | CATTCACTGAATACACCATCCACTGGGTGAGGCAGGCCCCT |
| | | GGAAAGGGCCTTGAGTGGATTGGAAACATTAATCCTAACAA |
| | | TGGTGGTACTACCTACAACCAGAAGTTCGAGGACAGAGTCA |
| | | CAATCACTGTAGACAAGTCCACCAGCACAGCCTACATGGAG |
| | | CTCAGCAGCCTGAGATCTGAGGATACTGCAGTCTATTACTG |
| | | TGCAGCTGGTTGGAACTTTGACTACTGGGGCCAAGGCACCA |
| | | CGGTCACCGTCTCCTCAGGAGGCGGAGGATCTGGCGGCGGA |
| | | GGAAGTTCTGGCGGAGGCAGCGACATTCAGATGACCCAGTC |
| | | TCCCAGCACCCTGTCCGCATCAGTAGGAGACAGGGTCACCA |
| | | TCACTTGCAAGGCCAGTCAGGATGTGGGTACTGCTGTAGAC |
| | | TGGTATCAACAGAAACCAGGGCAAGCTCCTAAACTACTGAT |
| | | TTACTGGGCATCCACCCGGCACACTGGAGTCCCTGATCGCT |
| | | TCAGCGGCAGTGGATCTGGGACAGATTTCACTCTCACCATC |
| | | AGCAGACTGCAGCCTGAAGACTTTGCAGTTTATTACTGTCA |
| | | GCAATATAACAGCTATCCTCTCACGTTCGGCCAGGGGACCA |
| | | AGGTGGATATCAAA |
| 75 | Mesothelin-specific | QVQLQQSGAEVKKPGASVKVSCKASGYTFTGYYMHWVRQAP |
| | binding domain | GQGLEWMGWINPNSGGTNYAQNFQGRVTMTRDTSISTAYME |
| | amino acid sequence | LRRLRSDDTAVYYCASGWDFDYWGQGTLVTVSSGGGGSGGG |
| | | GSGGGGSGGGGSDIRMTQSPSSLSASVGDRVTITCRASQSI |
| | | RYYLSWYQQKPGKAPKLLIYTASILQNGVPSRFSGSGSGTD |
| | | FTLTISSLQPEDFATYYCLQTYTTPDFGPGTKVEIK |
| 76 | TGFPRII dominant | MGRGLLRGLWPLHIVLWTRIASTIPPHVOKSVNNDMIVTDN |
| | negative receptor | NGAVKFPQLCKFCDVRFSTCDNQKSCMSNCSITSICEKPQE |
| | amino acid sequence | VCVAVWRKNDENITLETVCHDPKLPYHDFILEDAASPKCIM |
| | (TGFbRII-DN) | KEKKKPGETFFMCSCSSDECNDNIIFSEEYNTSNPDLLLVI |
| | | FQVTGISLLPPLGVAISVIIIFYCYRVNRQQKLSSSG |
| 77 | TGFPRII dominant | ATGGGTCGGGGGCTGCTCAGGGGCCTGTGGCCGCTGCACAT |
| | negative receptor | CGTCCTGTGGACGCGTATCGCCAGCACGATCCCACCGCACG |
| | nucleic acid sequence | TTCAGAAGTCGGTTAATAACGACATGATAGTCACTGACAAC |
| | (TGFbRII-DN) | AACGGTGCAGTCAAGTTTCCACAACTGTGTAAATTTTGTGA |
| | | TGTGAGATTTTCCACCTGTGACAACCAGAAATCCTGCATGA |
| | | GCAACTGCAGCATCACCTCCATCTGTGAGAAGCCACAGGAA |
| | | GTCTGTGTGGCTGTATGGAGAAAGAATGACGAGAACATAAC |
| | | ACTAGAGACAGTTTGCCATGACCCCAAGCTCCCCTACCATG |
| | | ACTTTATTCTGGAAGATGCTGCTTCTCCAAAGTGCATTATG |
| | | AAGGAAAAAAAAAAGCCTGGTGAGACTTTCTTCATGTGTTC |
| | | CTGTAGCTCTGATGAGTGCAATGACAACATCATCTTCTCAG |
| | | AAGAATATAACACCAGCAATCCTGACTTGTTGCTAGTCATA |
| | | TTTCAAGTGACAGGCATCAGCCTCCTGCCACCACTGGGAGT |
| | | TGCCATATCTGTCATCATCATCTTCTACTGCTACCGCGTTA |
| | | ACCGGCAGCAGAAGCTGAGTTCATCCGGA |
| 78 | PD1-CTM-CD28 receptor | MQIPQAPWPVVWAVLQLGWRPGWFLDSPDRPWNPPTFSPAL |
| | amino acid sequence | LVVTEGDNATFTCSFSNTSESFVLNWYRMSPSNQTDKLAAF |
| | | PEDRSQPGQDCRFRVTQLPNGRDFHMSVVRARRNDSGTYLC |
| | | GAISLAPKAQIKESLRAELRVTERRAEVPTAHPSPSPRPAG |
| | | QFQTLVFWVLVVVGGVLACYSLLVTVAFIIFWVRSKRSRLL |
| | | HSDYMNMTPRRPGPTRKHYQPYAPPRDFAAYRS |
| 79 | PD1-CTM-CD28 receptor | ATGCAGATCCCACAGGCGCCCTGGCCAGTCGTCTGGGCGGT |
| | nucleic acid sequence | GCTACAACTGGGCTGGCGGCCAGGATGGTTCTTAGACTCCC |
| | | CAGACAGGCCCTGGAACCCCCCCACCTTCTCCCCAGCCCTG |
| | | CTCGTGGTGACCGAAGGGGACAACGCCACCTTCACCTGCAG |
| | | CTTCTCCAACACATCGGAGAGCTTCGTGCTAAACTGGTACC |
| | | GCATGAGCCCCAGCAACCAGACGGACAAGCTGGCCGCCTTC |
| | | CCCGAGGACCGCAGCCAGCCCGGCCAGGACTGCCGCTTCCG |
| | | TGTCACACAACTGCCCAACGGGCGTGACTTCCACATGAGCG |
| | | TGGTCAGGGCCCGGCGCAATGACAGCGGCACCTACCTCTGT |
| | | GGGGCCATCTCCCTGGCCCCCAAGGCGCAGATCAAAGAGAG |
| | | CCTGCGGGCAGAGCTCAGGGTGACAGAGAGAAGGGCAGAAG |
| | | TGCCCACAGCCCACCCCAGCCCCTCACCCAGGCCAGCCGGC |
| | | CAGTTCCAAACCCTGGTGTTTTGGGTGCTGGTGGTGGTTGG |
| | | TGGAGTCCTGGCTTGCTATAGCTTGCTAGTAACAGTGGCCT |
| | | TTATTATTTTCTGGGTGAGGAGTAAGAGGAGCAGGCTCCTG |
| | | CACAGTGACTACATGAACATGACTCCCCGCCGCCCCGGGCC |
| | | CACCCGCAAGCATTACCAGCCCTATGCCCCACCACGCGACT |
| | | TCGCAGCCTATCGCTCC |
| 80 | PD1-PTM-CD28 receptor | MQIPQAPWPVVWAVLQLGWRPGWFLDSPDRPWNPPTFSPAL |
| | amino acid sequence | LVVTEGDNATFTCSFSNTSESFVLNWYRMSPSNQTDKLAAF |
| | | PEDRSQPGQDCRFRVTQLPNGRDFHMSVVRARRNDSGTVLC |
| | | GAISLAPKLQIKESLRAELRVTERRAEVPTAHPSPSPRPAG |
| | | QFQTLVVGVVGGLLGSLVLLVWVLAVIRSKRSRLLHSDYMN |
| | | MTPRRPGPTRKHYQPYAPPRDFAAVRS |
| 81 | PD1-PTM-CD28 receptor | ATGCAGATCCCACAGGCGCCCTGGCCAGTCGTCTGGGCGGT |
| | nucleic acid sequence | GCTACAACTGGGCTGGCGGCCAGGATGGTTCTTAGACTCCC |
| | | CAGACAGGCCCTGGAACCCCCCCACCTTCTCCCCAGCCCTG |
| | | CTCGTGGTGACCGAAGGGGACAACGCCACCTTCACCTGCAG |
| | | CTTCTCCAACACATCGGAGAGCTTCGTGCTAAACTGGTACC |
| | | GCATGAGCCCCAGCAACCAGACGGACAAGCTGGCCGCCTTC |
| | | CCCGAGGACCGCAGCCAGCCCGGCCAGGACTGCCGCTTCCG |
| | | TGTCACACAACTGCCCAACGGGCGTGACTTCCACATGAGCG |
| | | TGGTCAGGGCCCGGCGCAATGACAGCGGCACCTACCTCTGT |
| | | GGGGCCATCTCCCTGGCCCCCAAGGCGCAGATCAAAGAGAG |
| | | CCTGCGGGCAGAGCTCAGGGTGACAGAGAGAAGGGCAGAAG |
| | | TGCCCACAGCCCACCCCAGCCCCTCACCCAGGCCAGCCGGC |
| | | CAGTTCCAAACCCTGGTGGTTGGTGTCGTGGGCGGCCTGCT |
| | | GGGCAGCCTGGTGCTGCTAGTCTGGGTCCTGGCCGTCATCA |
| | | GGAGTAAGAGGAGCAGGCTCCTGCACAGTGACTACATGAAC |
| | | ATGACTCCCCGCCGCCCCGGGCCCACCCGCAAGCATTACCA |
| | | GCCCTATGCCCCACCACGCGACTTCGCAGCCTATCGCTCC |
| 82 | PD1A^{132L}_PTM-CD28 | MQIPQAPWPVVWAVLQLGWRPGWFLDSPDRPWNPPTFSPAL |
| | receptor | LVVTEGDNATFTCSFSNTSESFVLNWYRMSPSNQTDKLAAF |
| | amino acid sequence | PEDRSQPGQDCRFRVTQLPNGRDFHMSVVRARRNDSGTVLC |
| | | GAISLAPKLQIKESLRAELRVTERRAEVPTAHPSPSPRPAG |
| | | QFQTLVVGVVGGLLGSLVLLVWVLAVIRSKRSRLLHSDYMN |
| | | MTPRRPGPTRKHYQPYAPPRDFAAVRS |
| 83 | PD1^{A132L}_PTM-CD28 | ATGCAGATCCCACAGGCGCCCTGGCCAGTCGTCTGGGCGGT |
| | receptor | GCTACAACTGGGCTGGCGGCCAGGATGGTTCTTAGACTCCC |
| | nucleic acid sequence | CAGACAGGCCCTGGAACCCCCCCACCTTCTCCCCAGCCCTG |
| | | CTCGTGGTGACCGAAGGGGACAACGCCACCTTCACCTGCAG |
| | | CTTCTCCAACACATCGGAGAGCTTCGTGCTAAACTGGTACC |
| | | GCATGAGCCCCAGCAACCAGACGGACAAGCTGGCCGCCTTC |
| | | CCCGAGGACCGCAGCCAGCCCGGCCAGGACTGCCGCTTCCG |
| | | TGTCACACAACTGCCCAACGGGCGTGACTTCCACATGAGCG |
| | | TGGTCAGGGCCCGGCGCAATGACAGCGGCACCTACCTCTGT |
| | | GGGGCCATCTCCCTGGCCCCCAAGCTGCAGATCAAAGAGAG |
| | | CCTGCGGGCAGAGCTCAGGGTGACAGAGAGAAGGGCAGAAG |
| | | TGCCCACAGCCCACCCCAGCCCCTCACCCAGGCCAGCCGGC |
| | | CAGTTCCAAACCCTGGTGGTTGGTGTCGTGGGCGGCCTGCT |
| | | GGGCAGCCTGGTGCTGCTAGTCTGGGTCCTGGCCGTCATCA |
| | | GGAGTAAGAGGAGCAGGCTCCTGCACAGTGACTACATGAAC |
| | | ATGACTCCCCGCCGCCCCGGGCCCACCCGCAAGCATTACCA |
| | | GCCCTATGCCCCACCACGCGACTTCGCAGCCTATCGC |
| 84 | PD-1-4-1BB receptor | MQIPQAPWPVVWAVLQLGWRPGWFLDSPDRPWNPPTFSPAL |
| | amino acid sequence | LVVTEGDNATFTCSFSNTSESFVLNWYRMSPSNQTDKLAAF |
| | (PD1-BB) | PEDRSQPGQDCRFRVTQLPNGRDFHMSVVRARRNDSGTYLC |
| | | GAISLAPKAQIKESLRAELRVTERRAEVPTAHPSPSPRPAG |
| | | QFQTLVIYIWAPLAGTCGVLLLSLVITLYCKKRGRKKLLYI |
| | | FKQPFMRPVQTTQEEDGCSCRFPEEEEGGCEL |
| 85 | PD-1-4-1BB receptor | ATGCAGATCCCACAGGCGCCCTGGCCAGTCGTCTGGGCGGT |
| | nucleic acid sequence | GCTACAACTGGGCTGGCGGCCAGGATGGTTCTTAGACTCCC |
| | (PD1-BB) | CAGACAGGCCCTGGAACCCCCCCACCTTCTCCCCAGCCCTG |
| | | CTCGTGGTGACCGAAGGGGACAACGCCACCTTCACCTGCAG |
| | | CTTCTCCAACACATCGGAGAGCTTCGTGCTAAACTGGTACC |
| | | GCATGAGCCCCAGCAACCAGACGGACAAGCTGGCCGCCTTC |
| | | CCCGAGGACCGCAGCCAGCCCGGCCAGGACTGCCGCTTCCG |
| | | TGTCACACAACTGCCCAACGGGCGTGACTTCCACATGAGCG |
| | | TGGTCAGGGCCCGGCGCAATGACAGCGGCACCTACCTCTGT |
| | | GGGGCCATCTCCCTGGCCCCCAAGGCGCAGATCAAAGAGAG |
| | | CCTGCGGGCAGAGCTCAGGGTGACAGAGAGAAGGGCAGAAG |
| | | TGCCCACAGCCCACCCCAGCCCCTCACCCAGGCCAGCCGGC |
| | | CAGTTCCAAACCCTGGTTATCTACATCTGGGCGCCCTTGGC |
| | | CGGGACTTGTGGGGTCCTTCTCCTGTCACTGGTTATCACCC |
| | | TTTACTGCAAAAAACGGGGCAGAAAGAAACTCCTGTATATA |
| | | TTCAAACAACCATTTATGAGACCAGTACAAACTACTCAAGA |
| | | GGAAGATGGCTGTAGCTGCCGATTTCCAGAAGAAGAAGAAG |
| | | GAGGATGTGAACTG |
| 86 | PD1^{A132L}_4-1BB receptor | MQIPQAPWPVVWAVLQLGWRPGWFLDSPDRPWNPPTFSPAL |
| | amino acid sequence | LVVTEGDNATFTCSFSNTSESFVLNWYRMSPSNQTDKLAAF |
| | (PD1*BB) | PEDRSQPGQDCRFRVTQLPNGRDFHMSVVRARRNDSGTVLC |
| | | GAISLAPKLQIKESLRAELRVTERRAEVPTAHPSPSPRPAG |
| | | QFQTLVIYIWAPLAGTCGVLLLSLVITLVCKKRGRKKLLYI |
| | | FKQPFMRPVQTTQEEDGCSCRFPEEEEGGCEL |
| 87 | PD1^{A132L}_4-1BB receptor | ATGCAGATCCCACAGGCGCCCTGGCCAGTCGTCTGGGCGGT |
| | nucleic acid sequence | GCTACAACTGGGCTGGCGGCCAGGATGGTTCTTAGACTCCC |
| | (PD1*BB) | CAGACAGGCCCTGGAACCCCCCCACCTTCTCCCCAGCCCTG |
| | | CTCGTGGTGACCGAAGGGGACAACGCCACCTTCACCTGCAG |
| | | CTTCTCCAACACATCGGAGAGCTTCGTGCTAAACTGGTACC |
| | | GCATGAGCCCCAGCAACCAGACGGACAAGCTGGCCGCCTTC |
| | | CCCGAGGACCGCAGCCAGCCCGGCCAGGACTGCCGCTTCCG |
| | | TGTCACACAACTGCCCAACGGGCGTGACTTCCACATGAGCG |
| | | TGGTCAGGGCCCGGCGCAATGACAGCGGCACCTACCTCTGT |
| | | GGGGCCATCTCCCTGGCCCCCAAGCTGCAGATCAAAGAGAG |
| | | CCTGCGGGCAGAGCTCAGGGTGACAGAGAGAAGGGCAGAAG |
| | | TGCCCACAGCCCACCCCAGCCCCTCACCCAGGCCAGCCGGC |
| | | CAGTTCCAAACCCTGGTTATCTACATCTGGGCGCCCTTGGC |
| | | CGGGACTTGTGGGGTCCTTCTCCTGTCACTGGTTATCACCC |
| | | TTTACTGCAAAAAACGGGGCAGAAAGAAACTCCTGTATATA |
| | | TTCAAACAACCATTTATGAGACCAGTACAAACTACTCAAGA |
| | | GGAAGATGGCTGTAGCTGCCGATTTCCAGAAGAAGAAGAAG |
| | | GAGGATGTGAACTG |
| 88 | TGFβR-IL12Rβ1 receptor | MEAAVAAPRPRLLLLVLAAAAAAAAALLPGATALQCFCHLC |
| | amino acid sequence | TKDNFTCVTDGLCFVSVTETTDKVIHNSMCIAEIDLIPRDR |
| | | PFVCAPSSKTGSVTTTYCCNQDHCNKIELPTTVKSSPGLGP |
| | | VELAAVIAGPVCFVCISLMLMVYIRAARHLCPPLPTPCASS |
| | | AIEFPGGKETWOWINPVDFQEEASLQEALVVEMSWDKGERT |
| | | EPLEKTELPEGAPELALDTELSLEDGDRCKAKM |
| 89 | TGFβR-IL12Rβ1 receptor | ATGGAGGCGGCGGTCGCTGCTCCGCGTCCCCGGCTGCTCCT |
| | nucleic acid sequence | CCTCGTGCTGGCGGCGGCGGCGGCGGCGGCGGCGGCGCTGC |
| | | TCCCGGGGGCGACGGCGTTACAGTGTTTCTGCCACCTCTGT |
| | | ACAAAAGACAATTTTACTTGTGTGACAGATGGGCTCTGCTT |
| | | TGTCTCTGTCACAGAGACCACAGACAAAGTTATACACAACA |
| | | GCATGTGTATAGCTGAAATTGACTTAATTCCTCGAGATAGG |
| | | CCGTTTGTATGTGCACCCTCTTCAAAAACTGGGTCTGTGAC |
| | | TACAACATATTGCTGCAATCAGGACCATTGCAATAAAATAG |
| | | AACTTCCAACTACTGTAAAGTCATCACCTGGCCTTGGTCCT |
| | | GTGGAACTGGCAGCTGTCATTGCTGGACCAGTGTGCTTCGT |
| | | CTGCATCTCACTCATGTTGATGGTCTATATCAGGGCCGCAC |
| | | GGCACCTGTGCCCGCCGCTGCCCACACCCTGTGCCAGCTCC |
| | | GCCATTGAGTTCCCTGGAGGGAAGGAGACTTGGCAGTGGAT |
| | | CAACCCAGTGGACTTCCAGGAAGAGGCATCCCTGCAGGAGG |
| | | CCCTGGTGGTAGAGATGTCCTGGGACAAAGGCGAGAGGACT |
| | | GAGCCTCTCGAGAAGACAGAGCTACCTGAGGGTGCCCCTGA |
| | | GCTGGCCCTGGATACAGAGTTGTCCTTGGAGGATGGAGACA |
| | | GGTGCAAGGCCAAGATG |
| 90 | TGFβR-IL12Rβ2 receptor | MGRGLLRGLWPLHIVLWTRIASTIPPHVOKSVNNDMIVTDN |
| | amino acid sequence | NGAVKFPQLCKFCDVRFSTCDNQKSCMSNCSITSICEKPQE |
| | | VCVAVWRKNDENITLETVCHDPKLPYHDFILEDAASPKCIM |
| | | KEKKKPGETFFMCSCSSDECNDNIIFSEEYNTSNPDLLLVI |
| | | FQVTGISLLPPLGVAISVIIIFYQQKVFVLLAALRPOWCSR |
| | | EIPDPANSTCAKKYPIAEEKTOLPLDRLLIDWPTPEDPEPL |
| | | VISEVLHQVTPVFRHPPCSNWPQREKGIQGHQASEKDMMHS |
| | | ASSPPPPRALQAESRQLVDLYKVLESRGSDPKPENPACPWT |
| | | VLPAGDLPTHDGYLPSNIDDLPSHEAPLADSLEELEPQHIS |
| | | LSVFPSSSLHPLTFSCGDKLTLDQLKMRCDSLML |
| 91 | TGFβR-IL12Rβ2 receptor | ATGGGTCGGGGGCTGCTCAGGGGCCTGTGGCCGCTGCACAT |
| | nucleic acid sequence | CGTCCTGTGGACGCGTATCGCCAGCACGATCCCACCGCACG |
| | | TTCAGAAGTCGGTTAATAACGACATGATAGTCACTGACAAC |
| | | AACGGTGCAGTCAAGTTTCCACAACTGTGTAAATTTTGTGA |
| | | TGTGAGATTTTCCACCTGTGACAACCAGAAATCCTGCATGA |
| | | GCAACTGCAGCATCACCTCCATCTGTGAGAAGCCACAGGAA |
| | | GTCTGTGTGGCTGTATGGAGAAAGAATGACGAGAACATAAC |
| | | ACTAGAGACAGTTTGCCATGACCCCAAGCTCCCCTACCATG |
| | | ACTTTATTCTGGAAGATGCTGCTTCTCCAAAGTGCATTATG |
| | | AAGGAAAAAAAAAAGCCTGGTGAGACTTTCTTCATGTGTTC |
| | | CTGTAGCTCTGATGAGTGCAATGACAACATCATCTTCTCAG |
| | | AAGAATATAACACCAGCAATCCTGACTTGTTGCTAGTCATA |
| | | TTTCAAGTGACAGGCATCAGCCTCCTGCCACCACTGGGAGT |
| | | TGCCATATCTGTCATCATCATCTTCTACCAGCAAAAGGTGT |
| | | TTGTTCTCCTAGCAGCCCTCAGACCTCAGTGGTGTAGCAGA |
| | | GAAATTCCAGATCCAGCAAATAGCACTTGCGCTAAGAAATA |
| | | TCCCATTGCAGAGGAGAAGACACAGCTGCCCTTGGACAGGC |
| | | TCCTGATAGACTGGCCCACGCCTGAAGATCCTGAACCGCTG |
| | | GTCATCAGTGAAGTCCTTCATCAAGTGACCCCAGTTTTCAG |
| | | ACATCCCCCCTGCTCCAACTGGCCACAAAGGGAAAAAGGAA |
| | | TCCAAGGTCATCAGGCCTCTGAGAAAGACATGATGCACAGT |
| | | GCCTCAAGCCCACCACCTCCAAGAGCTCTCCAAGCTGAGAG |
| | | CAGACAACTGGTGGATCTGTACAAGGTGCTGGAGAGCAGGG |
| | | GCTCCGACCCAAAGCCAGAAAACCCAGCCTGTCCCTGGACG |
| | | GTGCTCCCAGCAGGTGACCTTCCCACCCATGATGGCTACTT |
| | | ACCCTCCAACATAGATGACCTCCCCTCACATGAGGCACCTC |
| | | TCGCTGACTCTCTGGAAGAACTGGAGCCTCAGCACATCTCC |
| | | CTTTCTGTTTTCCCCTCAAGTTCTCTTCACCCACTCACCTT |
| | | CTCCTGTGGTGATAAGCTGACTCTGGATCAGTTAAAGATGA |
| | | GGTGTGACTCCCTCATGCTC |

B. Additional Antigen-Binding Polypeptides

In some embodiments, the modified T cell expresses an antigen-binding polypeptide, a cell surface receptor ligand, or a polypeptide that binds to a tumor antigen. In some instances, the antigen-binding domain comprises an antibody that recognizes a cell surface protein or a receptor expressed on a tumor cell. In some instances, the antigen-binding domain comprises an antibody that recognizes a tumor antigen. In some instances, the antigen-binding domain comprises a full length antibody or an antigen-binding fragment thereof, a Fab, a F(ab)₂, a monospecific Fab₂, a bispecific Fab₂, a trispecific Fab₂, a single-chain variable fragment (scFv), a diabody, a triabody, a minibody, a V-NAR, or a VhH.

C. Cell Surface Receptor Ligands

In some embodiments, the modified T cell expresses a cell surface receptor ligand. In some instances, the ligand binds to a cell surface receptor expressed on a tumor cell. In some cases, the ligand comprises a wild-type protein or a variant thereof that binds to the cell surface receptor. In some instances, the ligand comprises a full-length protein or a functional fragment thereof that binds to the cell surface receptor. In some cases, the functional fragment comprises about 90%, about 80%, about 70%, about 60%, about 50%, or about 40% in length as compared to the full length version of the protein but retains binding to the cell surface receptor. In some cases, the ligand is a de novo engineered protein that binds to the cell surface receptor. Exemplary ligands include, but are not limited to, epidermal growth factor (EGF), platelet-derived growth factor (PDGF), or Wnt3A.

D. Tumor Antigens

In some embodiments, the modified T cell expresses a polypeptide that binds to a tumor antigen. In some instances, the tumor antigen is associated with a hematologic malignancy. Exemplary tumor antigens include, but are not limited to, CD19, CD20, CD22, CD33/IL3Ra, ROR1, mesothelin, c-Met, PSMA, PSCA, Folate receptor alpha, Folate receptor beta, EGFRvIII, GPC2, Tn-MUC1, GDNF family receptor alpha-4 (GFRa4), fibroblast activation protein (FAP), and IL13Ra2. In some instances, the tumor antigen comprises CD19, CD20, CD22, BCMA, CD37, Mesothelin, PSMA, PSCA, Tn-MUC1, EGFR, EGFRvIII, c-Met, HER1, HER2, CD33, CD133, GD2, GPC2, GPC3, NKG2D, KRAS, or WT1. In some instances, the polypeptide is a ligand of the tumor antigen, e.g., a full-length protein that binds to the tumor antigen, a functional fragment thereof, or a de novo engineered ligand that binds to the tumor antigen. In some instances, the polypeptide is an antibody that binds to the tumor antigen.

E. Switch Receptors and Dominant Negative Receptors

In one aspect, the present disclosure also includes a modified immune cell with downregulated gene expression as described herein further comprising an exogenous nucleic acid encoding a dominant negative receptor, a switch receptor, or a combination thereof. In some embodiments, the modified immune cell with downregulated gene expression as described herein further comprises a chimeric antigen receptor (CAR), and/or a dominant negative receptor. In some embodiments, the modified immune cell with downregulated gene expression as described herein further comprises a CAR, and a switch receptor. In some embodiments, the modified immune cell with downregulated gene expression as described herein further comprises an engineered TCR, and a switch receptor. In some embodiments, the modified immune cell with downregulated gene expression as described herein further comprises an engineered TCR, and a dominant negative receptor. In some embodiments, the modified immune cell with downregulated gene expression as described herein further comprises a KIR, and a switch receptor. In some embodiments, the modified immune cell with downregulated gene expression as described herein further comprises a KIR, and a dominant negative receptor.

1. Switch Receptor

The present invention provides compositions and methods for modified immune cells or precursors thereof with downregulated gene expression comprising a CAR and a switch receptor. Tumor cells generate an immunosuppressive microenvironment that serves to protect them from immune recognition and elimination. This immunosuppressive microenvironment can limit the effectiveness of immunosuppressive therapies such as CAR-T or TCR-T cell therapy. For example, the secreted cytokine Transforming Growth Factor β (TGF β) directly inhibits the function of cytotoxic T cells and additionally induces regulatory T cell formation to further suppress immune responses. T cell immunosuppression due to TGFβ in the context of prostate cancers has been previously demonstrated by Donkor et al (2011), and Shalapour et al (2015). To reduce the immunosuppressive effects of TGF on the immune cells can be modified to express an engineered TGFβR comprising the extracellular ligand-binding domain of the TGFβR fused to the intracellular signaling domain of, for example, Interleukin-12 receptor (IL12R; TGFβR-IL12R). Therefore, a modified immune cell comprising a switch receptor may bind a negative signal transduction molecule in the microenvironment of the modified immune cell, and convert the negative signal transduction signal of an inhibitory molecule may have on the modified immune cell into a positive signal that stimulate the modified immune cell. A switch receptor of the present invention may be designed to reduce the effects of a negative signal transduction molecule, or to convert the negative signal into a positive signal, by virtue of comprising an intracellular domain associated with the positive signal.

Thus, in some embodiments, the modified immune cell comprising an insertion and/or deletion in one or more gene loci encoding an endogenous immune protein has been further genetically modified to express a switch receptor. As used herein, the term “switch receptor” refers to a molecule designed to reduce the effect of a negative signal transduction molecule on a modified immune cell of the present invention. The switch receptor comprises: a first domain that is derived from a first polypeptide that is associated with a negative signal (a signal transduction that suppresses or inhibits a cell or T cell activation); and a second domain that is derived from a second polypeptide that is associated with a positive signal (a signal transduction signal that stimulate a cell or a T cell). In some embodiments, the protein associated with the negative signal is selected from the group consisting of CTLA4, PD-1, TGFβRII, BTLA, VSIG3, VSIG8, and TIM-3. In some embodiments, the protein associated with the positive signal is selected from the group consisting of CD28, 4-1BB, IL12Rβ1, IL12Rβ2, CD2, ICOS, and CD27.

In one embodiment, the first domain comprises at least a portion of the extracellular domain of the first polypeptide that is associated with a negative signal, and the second domain comprises at least a portion of the intracellular domain of the second polypeptide that is associated with a positive signal. As such, a switch receptor comprises an extracellular domain associated with a negative signal fused to an intracellular domain associated with a positive signal. In some embodiments, the switch receptor comprises an extracellular domain of a signaling protein associated with a negative signal, a transmembrane domain, and an intracellular domain of a signaling protein associated with a positive signal. In some embodiments, the transmembrane domain of the switch receptor is selected from the transmembrane of the protein associated with a negative signal or the transmembrane domain of the protein associated with the negative signal. In some embodiments, the transmembrane domain of the switch receptor is selected from a transmembrane domain of a protein selected from the group consisting of CTLA4, PD-1, VSIG3, VSIG8, TGFβRII, BTLA, TIM-3, CD28, 4-1BB, IL12Rβ1, IL12Rβ2, CD2, ICOS, and CD27.

In some embodiments, the switch receptor is selected from the group consisting of PD-1-CD28, PD-1^{A132L}-CD28, PD-1-CD27, PD-1^{A132L}-CD27, PD-1-4-1BB, PD-1^{A132L}-4-1BB, PD-1-ICOS, PD-1^{A132L}-ICOS, PD-1-IL12Rβ1, PD-1A132L-IL12Rβ1, PD-1-IL12Rβ2, PD-1^{A132L}-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2.

In some embodiments, the switch receptor is PD-1-CD28 and comprises an amino acid sequence set forth in SEQ ID NO: 78. In one embodiments, the switch receptor is PD-1^{A132L}-CD28 and comprises an amino acid sequence set forth in SEQ ID NO: 82. In one embodiments, the switch receptor is PD-1-4-1BB and comprises an amino acid sequence set forth in SEQ ID NO: 84. In one embodiments, the switch receptor is PD-1^{A132L}-4-1BB and comprises an amino acid sequence set forth in SEQ ID NO: 86. In one embodiments, the switch receptor is TGFβRII-IL12Rβ1 and comprises an amino acid sequence set forth in SEQ ID NO: 88. In one embodiments, the switch receptor is TGFβRII-IL12Rβ2 and comprises an amino acid sequence set forth in SEQ ID NO: 90. In one embodiments, the switch receptor is encoded in a nucleic acid sequence set forth in SEQ ID NO: 79, 81, 83, 85, 87, 89, or 91.

Tolerable variations of the switch receptor will be known to those of skill in the art, while maintaining its intended biological activity (e.g., converting a negative signal into a positive signal when expressed in a cell). Accordingly, in some embodiments, the switch receptor of the present invention may be encoded by a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the nucleic acid sequence set forth in SEQ ID NO: 79, 81, 83, 85, 87, 89, or 91. In some embodiments, the switch receptor of the present invention may comprise an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 78, 80, 82, 84, 86, 88, or 90.

In some embodiments, the modified immune cell comprises an insertion and/or deletion that is capable of downregulating CD3, B2M, and CIITA, a CAR, and switch receptor selected from the group consisting of PD-1-CD28, PD-1^{A132L}-CD28, PD-1-CD27, PD-1^{A132L}-CD27, PD-1-4-1BB, PD-1^{A132L}-4-1BB, PD-1-ICOS, PD-1^{A132L}-ICOS, PD-1-IL12Rβ1, PD-1A132L-IL12Rβ1, PD-1-IL12Rβ2, PD-1^{A132L}-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2. In some embodiments, the modified immune cell comprises an insertion and/or deletion in one or more gene loci each encoding an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, invariant chain (Ii Chain), and a combination thereof, a CAR, and a switch receptor selected from the group consisting of PD-1-CD28, PD-1^{A132L}-CD28, PD-1-CD27, PD-1^{A132L}-CD27, PD-1-4-1BB, PD-1^{A132L}-4-1BB, PD-1-ICOS, PD-1^{A132L}-ICOS, PD-1-IL12Rβ1, PD-1A132L-IL12Rβ1, PD-1-IL12Rβ2, PD-1^{A132L}-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2.

2. Dominant Negative Receptor

The present invention provides compositions and methods for modified immune cells or precursors thereof with downregulated gene expression comprising a CAR and a dominant negative receptor. Thus, in some embodiments, the modified immune cell comprising an insertion and/or deletion in one or more gene loci encoding an endogenous immune protein has been further genetically modified to express a dominant negative receptor. As used herein, the term “dominant negative receptor” refers to a molecule designed to reduce the effect of a negative signal transduction molecule (e.g., the effect of a negative signal transduction molecule on a modified immune cell of the present invention). A dominant negative receptor is a truncated variant of a wild-type protein associated with a negative signal. In some embodiments, the protein associated with a negative signal he protein associated with the negative signal is selected from the group consisting of CTLA4, PD-1, BTLA, TGFβRII, VSIG3, VSIG8, and TIM-3.

A dominant negative receptor of the present invention may bind a negative signal transduction molecule (e.g., CTLA4, PD-1, BTLA, TGFβRII, VSIG3, VSIG8, and TIM-3) by virtue of an extracellular domain associated with the negative signal, may reduce the effect of the negative signal transduction molecule. For example, a modified immune cell comprising a dominant negative receptor may bind a negative signal transduction molecule in the microenvironment of the modified immune cell, but this binding will not transduce this signal inside the cell to modify the activity of the modified T cell. Rather, the binding sequesters the negative signal transduction molecule and prevents its binding to endogenous receptor/ligand, thereby reducing the effect of the negative signal transduction molecule may have on the modified immune cell. As such, to reduce the immunosuppressive effects of certain molecule, immune cells can be modified to express a dominant negative receptor that is a dominant negative receptor.

In some embodiments, the dominant negative receptor comprises a truncated variant of a wild-type protein associated with a negative signal. In some embodiments, the dominant negative receptor comprises a variant of a wild-type protein associated with a negative signal comprising an extracellular domain, a transmembrane domain, and substantially lacking an intracellular signaling domain. In some embodiments, the dominant negative receptor comprises an extracellular domain of a signaling protein associated with a negative signal, and a transmembrane domain. In some embodiments, the dominant negative receptor is PD-1, CTLA4, BTLA, TGFβRII, VSIG3, VSIG8, or TIM-3 dominant negative receptor. In some embodiments, the dominant negative receptor is PD-1, or TGFβRII. In some embodiments, the TGFβRII comprises an amino acid sequence set forth in SEQ ID NO: 76. In some embodiments, the TGFβRII is encoded by a nucleic acid sequence set forth in SEQ ID NO: 77.

Tolerable variations of the dominant negative receptor will be known to those of skill in the art, while maintaining its intended biological activity (e.g., blocking a negative signal and/or sequestering a molecule having a negative signal when expressed in a cell). Accordingly, in some embodiments, the dominant negative receptor of the present invention may be encoded by a nucleic acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to the nucleic acid sequence set forth in SEQ ID NO: 77. In some embodiments, the dominant negative receptor of the present invention may comprise an amino acid sequence that has at least at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% sequence identity to SEQ ID NO: 76.

F. Chemokine and Cytokine as Immune Enhancing Factors for Improved Fitness

The present invention provides compositions and methods for modified immune cells with downregulated immune gene expression comprising a CAR, and further comprising a dominant negative receptor, a switch receptor, a chemokine, a chemokine receptor, a cytokine, a cytokine receptor, Interleukin -7 (IL-7), Interleukin-7 receptor (IL-7R), Interleukin-15 (IL-15), Interleukin-15 receptor (IL-15R), Interleukin-21 (IL-21), Interleukin-18 (IL-18), CCL21, CCL19, or a combination thereof. In some embodiments, a chemokine, a chemokine receptor, a cytokine, a cytokine receptor, IL-7, IL-7R, IL-15, IL-15R, IL-21, IL-18, C—C Motif Chemokine Ligand 21 (CCL21), or C—C Motif Chemokine Ligand 19 (CCL19) is an immune function-enhancing factor that improves the fitness of the claimed modified immune cell. Without wishing to be bound by theory, the addition of a chemokine, a chemokine receptor, a cytokine, a cytokine receptor, IL-7, IL-7R, IL-15, IL-15R, IL-21, IL-18, CCL21, or CCL19 to the modified immune cell enhances the immunity-inducing effect and antitumor activity of the modified immune cell.

Without wishing to be bound by theory, interleukins and chemokines, may promote increase T cell priming and/or T cell infiltration in a solid tumor. For instance, in microsatellite stable colorectal cancers (CRCs) with low T cell infiltration, IL-15 promotes T cell priming. In some embodiments, the combination of a CAR and chemokine/interleukine receptor complex promotes T cell priming. Furthermore, IL-15 may induce NK cell infiltration. In some embodiments, response to an IL-15/IL-15RA complex can result in NK cell infiltration. In certain embodiments, the modified immune cell described herein further comprises an IL-15/IL-15Ra complex. In some embodiments, the IL-15/IL-15Ra complex is chosen from NIZ985 (Novartis), ATL-803 (Altor) or CYP0150 (Cytune). In some embodiments, the IL-15/IL-15RA complex is NIZ985. In some embodiments, IL-15 stimulates Natural Killer cells to eliminate (e.g., kill) pancreatic cancer cells. In some embodiments, therapeutic response to a modified immune cell described herein further comprising IL-15/IL15Ra is associated with Natural Killer cell infiltration in an animal model of colorectal cancer. In some embodiments, the IL-15/IL-15Ra complex comprises human IL-15 complexed with a soluble form of human IL-15Ra. The complex may comprise IL-15 covalently or noncovalently bound to a soluble form of IL-15Ra. In a particular embodiment, the human IL-15 is noncovalently bonded to a soluble form of IL-15Ra.

The ineffectiveness of CAR T cell therapy against solid tumors is partially caused by the limited recruitment and accumulation of immune cells and CAR T cells in solid tumors. One approach to solve this problem is to engineer CAR T cells that mimic the function of T-zone fibroblastic reticular cells (FRC). The lymph node is responsible for detecting pathogens and immunogens. The T-zone contains three types of cells: (1) innate immunity cells such as dendritic cells, monocytes, macrophages, and granulocytes; (2) adaptive immunity cells, such as CD4 and CD8 lymphocytes, and (3) stromal cells (FRCs). These cells cooperate to mount an effective immune response against a pathogen by facilitating the activation, differentiation and maturation of CD4 T cells. FRCs are particularly important because they form a network that allows dendritic cells and T cells to travel throughout the lymph node, and attracts B cells. In particular, FRCs provide a network for: (i) The recruitment of naive T cells, B cells and dendritic cells to the lymph node by releasing two chemokines (CCL21 and CCL19); (ii) T cell survival by secreting IL-7, which is a survival factor particularly for naive T cells; and (iii) trafficking of CD4 T cells toward the germinal center (GC; a different part of the lymph node). Accordingly, CAR armored with exogenous CCL21, or CCL19 and IL-7, will enhance the recruitment of T cells, B cells and dendritic cells to solid tumors. In some embodiments, the modified T cell comprises a nucleic acid encoding an immune function-enhancing factor, wherein the nucleic acid encoding an immune function-enhancing factor is a nucleic acid encoding interleukin-7 and a nucleic acid encoding CCL19 or CCL21.

In some embodiments, the nucleic acid of the immune function-enhancing factor (i.e. chemokine, the chemokine receptor, the cytokine, the cytokine receptor, IL-7, IL-7R, IL-15, IL-15R, IL-21, IL-18, CCL21, or CCL19) is fused to a CAR. In some embodiments, the chemokine, the chemokine receptor, the cytokine, the cytokine receptor, IL-7, IL-7R, IL-15, IL-15R, IL-21, IL-18, CCL21, or CCL19 is fused to a CAR via a self-cleaving peptide, such as a P2A, a T2A, an E2A, or an F2A.

### VI. Methods of Generating a Modified T Cell

One aspect of the present invention provides a method of generating a modified immune cell (e.g. an allogeneic T cell, NK cell, or NKT cell). The modified immune cell of the present invention is generally engineered by (1) introducing into the immune cell one or more nucleic acids capable of downregulating gene expression of one or more endogenous immune genes encoding an endogenous immune protein; (2) introducing into the immune cell an exogenous nucleic acid encoding an engineered receptor; and (3) expanding the modified immune cell to generate a modified immune T cells. Such a modified immune cell can be included in a therapeutic composition and administered to a patient in need thereof.

In some embodiments, a method for generating a modified immune cell of the present invention comprises introducing into the immune cell one or more nucleic acids capable of downregulating gene expression of one or more endogenous immune genes. The one or more immune genes encodes an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain). In addition, introducing into the immune cell an exogenous nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen is also introduced into the immune cell. In some embodiments, the method further comprises introducing into the immune cell an exogenous nucleic acid encoding a dominant negative receptor, a switch receptor, or a combination thereof.

A. Method of Introducing Nucleic Acid into a Cell

Methods of introducing nucleic acids into a cell include physical, biological and chemical methods. Physical methods for introducing a polynucleotide, such as RNA, into a host cell include calcium phosphate precipitation, lipofection, particle bombardment, microinjection, electroporation, and the like. RNA can be introduced into target cells using commercially available methods which include electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendorf, Hamburg Germany). RNA can also be introduced into cells using cationic liposome mediated transfection using lipofection, using polymer encapsulation, using peptide mediated transfection, or using biolistic particle delivery systems such as “gene guns.”

1. Biological Methods

Biological methods for introducing a polynucleotide of interest into a host cell (e.g. immune cell) include the use of DNA and RNA vectors. Viral vectors, and especially retroviral vectors, have become the most widely used method for inserting genes into mammalian (e.g., human cells). Other viral vectors can be derived from lentivirus, poxviruses, herpes simplex virus I, adenoviruses and adeno-associated viruses, and the like. See for example, U.S. Pat. Nos. 5,350,674 and 5,585,362.

In some embodiments, a nucleic acid encoding a subject CAR, a subject engineered TCR, a subject KIR, a subject antigen-binding polypeptide, a subject cell surface receptor ligand, a subject tumor antigen, a subject switch receptor, and/or a subject dominant negative receptor of the invention is introduced into a cell by an expression vector. Expression vectors comprising a nucleic acid encoding a subject CAR, a subject engineered TCR, a subject KIR, a subject antigen-binding polypeptide, a subject cell surface receptor ligand, a subject tumor antigen, a subject switch receptor, and/or a subject dominant negative receptor are provided herein. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggyback, and Integrases such as Phi31. Some other suitable expression vectors include herpes simplex virus (HSV) and retrovirus expression vectors.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the subject CAR, the subject engineered TCR, the subject KIR, the subject antigen-binding polypeptide, the subject cell surface receptor ligand, the subject tumor antigen, the subject switch receptor, and/or the subject dominant negative receptor in the host cell. In some embodiments, the adenovirus genome is a 36 kb, linear, double stranded DNA, where a foreign DNA sequence. For example, a nucleic acid encoding a subject CAR, a subject engineered TCR, a subject KIR, a subject antigen-binding polypeptide, a subject cell surface receptor ligand, a subject tumor antigen, a subject switch receptor, and/or a subject dominant negative receptor may be inserted to substitute large pieces of adenoviral DNA in order to make the expression vector of the present invention.

Another expression vector is based on an adeno associated virus, which takes advantage of the adenovirus coupled systems. This AAV expression vector has a high frequency of integration into the host genome. It can infect non-dividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retrovirus vector is constructed by inserting a nucleic acid (e.g., a nucleic acid encoding a subject CAR, a subject engineered TCR, a subject KIR, a subject antigen-binding polypeptide, a subject cell surface receptor ligand, a subject tumor antigen, a subject switch receptor, and/or a subject dominant negative receptor) into the viral genome at certain locations to produce a virus that is replication defective. Though the retrovirus vectors are able to infect a broad variety of cell types, integration and stable expression of the subject CAR, the subject engineered TCR, the subject KIR, the subject antigen-binding polypeptide, the subject cell surface receptor ligand, the subject tumor antigen, the subject switch receptor, and/or the subject dominant negative receptor, requires the division of host cells.

Lentivirus vectors are derived from lentiviruses, which are complex retroviruses that, in addition to the common retroviral genes gag, pol, and env, contain other genes with regulatory or structural function. See, e.g., U.S. Pat. Nos. 6,013,516 and 5,994,136. Some examples of lentiviruses include the human immunodeficiency viruses (HTV-1, HTV-2) and the simian immunodeficiency virus (SIV). Lentivirus vectors have been generated by multiply attenuating the HIV virulence genes, for example, the genes env, vif, vpr, vpu and nef are deleted making the vector biologically safe. Lentivirus vectors are capable of infecting non-dividing cells and can be used for both in vivo and ex vivo gene transfer and expression of a nucleic acid encoding a subject CAR, a subject engineered TCR, a subject KIR, a subject antigen-binding polypeptide, a subject cell surface receptor ligand, a subject tumor antigen, a subject switch receptor, and/or a subject dominant negative receptor. See, e.g., U.S. Pat. No. 5,994,136.

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell (e.g., immune cell) may be grown and expanded in culture before introduction of the expression vectors, followed by the appropriate treatment for introduction and integration of the vectors. The host cells (e.g., immune cells) are then expanded and may be screened by virtue of a marker present in the vectors. In some embodiments, the nucleic acids, encoding a subject CAR, a subject engineered TCR, a subject KIR, a subject antigen-binding polypeptide, a subject cell surface receptor ligand, a subject tumor antigen, a subject switch receptor, and/or a subject dominant negative receptor, are introduced into the immune cell by viral transduction. In some embodiments, the viral transduction comprises contacting the immune cell with a viral vector comprising the one or more nucleic acids. In some embodiments, the viral vector is selected from the group consisting of a retroviral vector, sendai viral vectors, adenoviral vectors, adeno-associated virus vectors, and lentiviral vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell is an immune cell or precursor thereof. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In some embodiments, the host cell is a T cell, an NK cell, or an NKT cell. In some embodiments, the immune cell is selected from the group consisting of a T cell, a natural killer cell (NK cell), a natural killer T cell, a lymphoid progenitor cell, a hematopoietic stem cell, a stem cell, a macrophage, and a dendritic cell. In some embodiments, the immune cell is a CD4+ T cell or a CD8+ T cell. In some embodiments, the immune cell is an allogeneic T cell or autologous T cell. In some embodiments, the allogeneic T cell or autologous T cell is human.

The modified immune cells of the present invention (e.g., comprising a nucleic acid capable of downregulating a gene, CAR, a KIR, a TCR a dominant negative receptor, and/or switch receptor) may be produced by stably transfecting host cells (e.g. immune cells) with an expression vector including a nucleic acid of the present disclosure. Additional methods to generate a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells (i.e. immune cells) expressing a nucleic acid capable of downregulating a gene, CAR, a KIR, a TCR a dominant negative receptor, and/or switch receptor of the present disclosure may be expanded ex vivo.

2. Physical Methods

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al., Molecular Cloning: A Laboratory Manual, Cold Spring Harbor Laboratory, New York (2001).

3. Chemical Methods

Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes. Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

Regardless of the method used to introduce exogenous nucleic acids into a host cell or otherwise expose a cell to the inhibitor of the present invention, to confirm the presence of the nucleic acids in the host cell, a variety of assays may be performed. Such assays include, for example, molecular biological assays well known to those of skill in the art, such as Southern and Northem blotting, RT-PCR and PCR; biochemical assays, such as detecting the presence or absence of a particular peptide (e.g., immunological means (ELISAs and Western blots) or by assays described herein to identify agents falling within the scope of the invention.

Moreover, the nucleic acids may be introduced by any means, such as transducing the expanded host cells (e.g., immune cells), transfecting the expanded host cells (e.g., immune cells), and electroporating the expanded host cells (e.g., immune cells). One nucleic acid may be introduced by one method and another nucleic acid may be introduced into the host cell (e.g., immune cells) by a different method.

4. RNA

In one embodiment, the nucleic acids introduced into the host cell (e.g., immune cell) are RNA. In another embodiment, the RNA is mRNA that comprises in vitro transcribed RNA or synthetic RNA. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be genomic DNA, plasmid DNA, phage DNA, cDNA, synthetic DNA sequence or any other appropriate source of DNA.

PCR can be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. As used herein, “Substantially Complementary” refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers can also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription. On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem, 270: 1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable. The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly (A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA. 5′ caps also provide stability to RNA molecules. In a preferred embodiment, RNAs produced by the methods disclosed herein include a 5′ cap. The 5′ cap is provided using techniques known in the art and described herein. Cougot, et al., Trends in Biochem. Sci. 29:436-444 (2001); Stepinski, et al, RNA 7: 1468-95 (2001); Elango, et al, Biochim. Biophys. Res. Commun. 330:958-966 (2005).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included. In some embodiments, the RNA is electroporated into the cells, such as in vitro transcribed RNA.

The disclosed methods can be applied to the modulation of host cell activity in basic research and therapy, in the fields of cancer, stem cells, acute and chronic infections, and autoimmune diseases, including the assessment of the ability of the genetically modified host cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains. One advantage of RNA transfection methods of the invention is that RNA transfection is essentially transient and a vector-free. A RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Accordingly, the present invention provides a method for generating a modified immune cell or precursor cell thereof comprising introducing into the immune cell one or more nucleic acids capable of downregulating gene expression of one or more endogenous immune genes as described herein, using any of the gene editing techniques described herein or known to those of skill in the art. Downregulating expression of an endogenous gene that is involved in producing an immune response to a cell, such as TCR a chain, TCR 0 chain, CD3δ, CD3ε, CD3γ, a HLA-I molecule (e.g., beta-2 microglobulin, TAP1, TAP2, TAPBP, or NLRC5) or a HLA-II molecule (e.g. CIITA, HLA-DM, RFX5, RFXANK, RFXAP, or Invariant chain), reduces immune-mediated rejection of the modified T cell. For example, downregulating expression of endogenous TCR receptor components, MHC-I or MHC-II, beta-2 microglobulin, CIITA genes removes surface presentation of alloantigens on the T cell that could cause rejection by the host immune system. In some embodiments, a nucleic acid capable of downregulating endogenous gene expression is introduced, such as by electroporation, transfection, or lenti- or other viral transduction, into the T cell. In some embodiments, the invention includes a modified T cell comprising an electroporated nucleic acid capable of downregulating endogenous gene expression. In some embodiments, the nucleic acids are introduced into the immune cell by viral transduction. In some embodiments, the viral transduction comprises contacting the immune cell with a viral vector comprising the one or more nucleic acids. In one embodiments, the viral vector is selected from the group consisting of a retroviral vector, sendai viral vectors, adenoviral vectors, adeno-associated virus vectors, and lentiviral vectors.

B. Method of Genetically Editing an Immune Cell

In one aspect, the present disclosure provides a method of genetically editing an immune cell comprising introducing into the immune cell one or more nucleic acids capable of downregulating gene expression of one or more endogenous immune genes encoding an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain). In one embodiment, a method of genetically editing a modified immune cell comprises introducing into the immune cell a nucleic acid capable of downregulating gene expression of a T cell receptor subunit selected from CD3δ, CD3ε, or CD3γ. In one embodiment, a method of genetically editing a modified immune cell comprises introducing into the immune cell a nucleic acid capable of downregulating gene expression of a HLA class I molecule selected from B2M, TAP1, TAP2, TAPBP, or NLRC5. In one embodiment, a method of genetically editing a modified immune cell comprises introducing into the immune cell a nucleic acid capable of downregulating gene expression of a HLA class II molecule selected from HLA-DM, RFX5, RFXANK, RFXAP, or invariant chain (Ii Chain).

In some embodiments, a method of genetically editing an immune cell comprises introducing into the immune cell a nucleic acid capable of downregulating gene expression of CD3δ, and the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof. In some embodiments, a method of genetically editing an immune cell comprises introducing into the immune cell a nucleic acid capable of downregulating gene expression of CD3ε, and the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof. In some embodiments, a method of genetically editing an immune cell comprises introducing into the immune cell a nucleic acid capable of downregulating gene expression of CD3γ, and the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof. In some embodiments, a method of genetically editing an immune cell comprises introducing into the immune cell a nucleic acid capable of downregulating gene expression of CD3γ, and the gene expression of CD3ε, B2M, and CIITA.

In some embodiments, a method of genetically editing an immune cell comprises introducing into the immune cell a nucleic acid capable of downregulating gene expression of: (1) CD3ε, B2M, and RFX5; (2) CD3ε, B2M, and RFXAP; (3) CD3ε, B2M, and RFXANK; (4) CD3ε, B2M, and HLA-DM; (5) CD3ε, B2M, and Ii chain; (6) CD3ε, TAP1, and CIITA; (7) CD3ε, TAP1, and RFX5; (8) CD3ε, TAP1, and RFXAP; (9) CD3 c, TAP1, and RFXANK; (10) CD3ε, TAP1, and HLA-DM; (11) CD3ε, TAP1, and Ii chain; (12) CD3ε, TAP2, and CIITA; (13) CD3ε, TAP2, and RFX5; (14) CD3ε, TAP2, and RFXAP; (15) CD3ε, TAP2, and RFXANK; (16) CD3ε, TAP2, and HLA-DM; (17) CD3ε, TAP2, and Ii chain; (18) CD3ε, NLRC5, and CIITA; (19) CD3ε, NLRC5, and RFX5; (20) CD3ε, NLRC5, and RFXAP; (21) CD3ε, NLRC5, and RFXANK; (22) CD3ε, NLRC5, and HLA-DM; (23) CD3ε, NLRC5, and Ii chain; (24) CD3ε, TAPBP, and CIITA; (25) CD3ε, TAPBP, and RFX5; (26) CD3ε, TAPBP, and RFXAP; (27) CD3ε, TAPBP, and RFXANK; (28) CD3ε, TAPBP, and HLA-DM; or (29) CD3ε, TAPBP, and Ii chain.

In some embodiments, a method of genetically editing an immune cell comprises introducing into the immune cell a nucleic acid capable of downregulating gene expression of: (1) CD3δ, B2M, and RFX5; (2) CD3δ, B2M, and RFXAP; (3) CD3δ, B2M, and RFXANK; (4) CD3δ, B2M, and HLA-DM; (5) CD3δ, B2M, and Ii chain; (6) CD3δ, TAP1, and CIITA; (7) CD3δ, TAP1, and RFX5; (8) CD3δ, TAP1, and RFXAP; (9) CD3δ, TAP1, and RFXANK; (10) CD3δ, TAP1, and HLA-DM; (11) CD3δ, TAP1, and Ii chain; (12) CD3δ, TAP2, and CIITA; (13) CD3δ, TAP2, and RFX5; (14) CD3δ, TAP2, and RFXAP; (15) CD3δ, TAP2, and RFXANK; (16) CD3δ, TAP2, and HLA-DM; (17) CD3δ, TAP2, and Ii chain; (18) CD3δ, NLRC5, and CIITA; (19) CD3δ, NLRC5, and RFX5; (20) CD3δ, NLRC5, and RFXAP; (21) CD3δ, NLRC5, and RFXANK; (22) CD3δ, NLRC5, and HLA-DM; (23) CD3δ, NLRC5, and Ii chain; (24) CD3δ, TAPBP, and CIITA; (25) CD3δ, TAPBP, and RFX5; (26) CD3δ, TAPBP, and RFXAP; (27) CD3δ, TAPBP, and RFXANK; (28) CD3δ, TAPBP, and HLA-DM; or (29) CD3δ, TAPBP, and Ii chain.

In some embodiments, a method of genetically editing an immune cell comprises introducing into the immune cell a nucleic acid capable of downregulating gene expression of: (1) CD3γ, B2M, and RFX5; (2) CD3γ, B2M, and RFXAP; (3) CD3γ, B2M, and RFXANK; (4) CD3γ, B2M, and HLA-DM; (5) CD3γ, B2M, and Ii chain; (6) CD3γ, TAP1, and CIITA; (7) CD3γ, TAP1, and RFX5; (8) CD3γ, TAP1, and RFXAP; (9) CD3γ, TAP1, and RFXANK; (10) CD3γ, TAP1, and HLA-DM; (11) CD3γ, TAP1, and Ii chain; (12) CD3γ, TAP2, and CIITA; (13) CD3γ, TAP2, and RFX5; (14) CD3γ, TAP2, and RFXAP; (15) CD3γ, TAP2, and RFXANK; (16) CD3γ, TAP2, and HLA-DM; (17) CD3γ, TAP2, and Ii chain; (18) CD3γ, NLRC5, and CIITA; (19) CD3γ, NLRC5, and RFX5; (20) CD3γ, NLRC5, and RFXAP; (21) CD3γ, NLRC5, and RFXANK; (22) CD3γ, NLRC5, and HLA-DM; (23) CD3γ, NLRC5, and Ii chain; (24) CD3γ, TAPBP, and CIITA; (25) CD3γ, TAPBP, and RFX5; (26) CD3γ, TAPBP, and RFXAP; (27) CD3γ, TAPBP, and RFXANK; (28) CD3γ, TAPBP, and HLA-DM or (29) CD3γ, TAPBP, and Ii chain.

In some embodiments, a method of genetically editing an immune cell comprising introducing into the immune cell a nucleic acid capable of downregulating gene expression comprising a gene editing system selected from the group consisting of an antisense RNA, antigomer RNA, siRNA, shRNA, and a CRISPR system. Endogenous immune gene expression may be downregulated, knocked-down, decreased, and/or inhibited by, for example, an antisense RNA, antigomer RNA, siRNA, shRNA, a CRISPR system, etc. In one embodiment, a method of genetically editing an immune cell comprises introducing into the immune cell a nucleic acid capable of downregulating gene expression comprising a CRISPR-associated (Cas) (CRISPR-Cas) endonuclease system and a guide RNA. In some embodiments, the nucleic acid capable of downregulating gene expression comprises a Cas endonuclease selected from the group consisting of Cas3, Cas4, Cas8a, Cas8b, Cas9, Cas10, Cas10d, Cas12a, Cas12b, Cas12d, Cas12e, Cas12f, Cas12g, Cas12h, Cas12i, Cas13, Cas14, CasX, Cse1, Csy1, Csn2, Cpf1, C2c1, Csm2, Cmr5, Fok1, S. pyogenes Cas9 (spCas9), Staphylococcus aureus Cas9 (saCas9), MAD7 nuclease (a type V CRISPR nuclease), and any combination thereof. Method of genetically editing a cell are well known in the art and described herein.

In some embodiments, a method of genetically editing an immune cell comprises introducing into the immune cell a CRISPR/Cas to disrupt one or more endogenous immune genes in a modified cell (e.g., a modified T cell). In some embodiments, CRISPR/Cas9 is used to disrupt one or more of endogenous an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain). In certain exemplary embodiments, CRISPR/Cas9 is used to disrupt one or more of endogenous CD3δ, CD3ε, CD3γ, B2M, CIITA, TAP1, TAP2, TAPBP, NLRC5, HLA-DM, RFX5, RFXANK, RFXAP, and invariant chain (Ii Chain), thereby resulting in the downregulation of CD3δ, CD3ε, CD3γ, B2M, CIITA, TAP1, TAP2, TAPBP, NLRC5, HLA-DM, RFX5, RFXANK, RFXAP, and invariant chain (Ii Chain). Suitable gRNAs for use in disrupting one or more of endogenous TRAC, TRBC, B2M, CIITA, and/or PD1 is set forth in FIGS. 26 and 27. In some embodiments, a method of genetically editing an immune cell comprises introducing into the immune cell a CRISPR/Cas and a guide RNA to disrupt one or more endogenous immune genes in a modified cell (e.g., a modified T cell). In one embodiment, a method of genetically editing an immune cell comprises introducing into the immune cell a CRISPR/Cas and a guide RNA, and the guide RNA comprises a guide sequence that is complementary with a sequence within the one or more gene loci selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain). Suitable guide RNAs (gRNAs) for use in disrupting one or more of endogenous CD3δ, CD3ε, CD3γ, B2M, CIITA, TAP1, TAP2, TAPBP, NLRC5, HLA-DM, RFX5, RFXANK, RFXAP, and invariant chain (Ii Chain) is set forth in Table 3.

In one embodiment, a method of genetically editing an immune cell comprising introducing into the immune cell a nucleic acid capable of downregulating gene expression comprising a TALEN gene editing system. In one embodiment, a method of genetically editing an immune cell comprising introducing into the immune cell a nucleic acid capable of downregulating gene expression comprising a zinc finger nuclease (ZFN) gene editing system. In one embodiment, a method of genetically editing an immune cell comprising introducing into the immune cell a nucleic acid capable of downregulating gene expression comprising a meganuclease gene editing system. In one embodiment, a method of genetically editing an immune cell comprising introducing into the immune cell a nucleic acid capable of downregulating gene expression comprising a mega-TALEN gene editing system. In one embodiment, a method of genetically editing a modified immune cell comprising introducing into the immune cell a nucleic acid capable of downregulating gene expression comprising a gene silencing system selected from antisense RNA, antigomer RNA, RNAi, siRNA, or shRNA.

C. Expansion of the Modified Immune Cells

In yet another embodiments, the method of generating the modified T cell as described herein further comprises expanding the modified immune cell to generate a population of modified T cells. Whether prior to or after modification of immune cells to express a CAR, TCR, a dominant negative receptor, and/or switch receptor, the modified cells can be activated and expanded in number using methods known in the art. For example, the immune cells of the invention may be expanded by contact with a surface having attached thereto an agent that stimulates a CD3/TCR complex associated signal and a ligand that stimulates a co-stimulatory molecule on the surface of the modified immune cells. In particular, the modified immune cell populations may be stimulated by contact with an anti-CD3 antibody, or an antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the modified immune cells, a ligand that binds the accessory molecule is used. For example, the modified immune cells can be contacted with an anti-CD3 antibody and an anti-CD28 antibody, under conditions appropriate for stimulating proliferation of the immune cells. Examples of an anti-CD28 antibody include 9.3, B-T3, XR-CD28 (Diaclone, Besancon, France) and these can be used in the invention, as can other methods and reagents known in the art.

Expanding the modified immune cells by the methods disclosed herein can be multiplied by about 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater, and any and all whole or partial integers there between. In one embodiment, the modified immune cells expand in the range of about 20 fold to about 50 fold.

Following culturing, the modified immune cells can be incubated in cell medium in a culture apparatus for a period of time or until the cells reach confluency or high cell density for optimal passage before passing the cells to another culture apparatus. The culturing apparatus can be of any culture apparatus commonly used for culturing cells in vitro. Preferably, the level of confluence is 70% or greater before passing the cells to another culture apparatus. More preferably, the level of confluence is 90% or greater. A period of time can be any time suitable for the culture of cells in vitro. The immune cell medium may be replaced during the culture of the immune cells at any time. Preferably, the immune cell medium is replaced about every 2 to 3 days. The immune cells are then harvested from the culture apparatus whereupon the modified immune cells can be used immediately or cryopreserved to be stored for use at a later time. In one embodiment, the invention includes cryopreserving the expanded modified immune cells. The cryopreserved immune cells are thawed prior to introducing nucleic acids into the immune cell.

In another embodiment, the method comprises isolating immune cells and expanding the immune cells. In another embodiment, the invention further comprises cryopreserving the immune cells prior to expansion. In yet another embodiment, the cryopreserved immune cells are thawed for electroporation with the RNA encoding the chimeric membrane protein.

In yet another embodiments, the method of generating the modified T cell as described herein further comprises expanding the modified immune cell ex vivo. In some embodiments, ex vivo culture and expansion of modified immune cells comprises the addition of cellular growth factors. However, other factors, such as flt3-L, IL-1, IL-3 and c-kit ligand can also be added. In some embodiments, expanding the modified T cell comprises culturing the modified T cell with a factor selected from the group consisting of flt3-L, IL-1, IL-3, IL-2, IL-7, IL-15, IL-18, IL-21, TGFbeta, IL-10, and c-kit ligand. The culturing step as described herein (contact with agents as described herein or after electroporation) can be very short, for example less than 24 hours such as 1, 2, 3, 4, 5, 6, 7, 8, 9, 10, 11 , 12, 13, 14, 15, 16, 17, 18, 19, 20, 21 , 22, or 23 hours. The culturing step can be longer, for example 1, 2, 3, 4, 5, 6, 7, 8, 9, 10, 11, 12, 13, 14, or more days.

Various terms are used to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Conditions appropriate for immune cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-gamma, IL-4, IL-7, GM-CSF, IL-10, IL-12, IL-15, TGF-beta, and TNF-a. or any other additives for the growth of cells known to the skilled artisan. Other additives for the growth of cells include, but are not limited to, surfactant, plasmanate, and reducing agents such as N-acetyl-cysteine and 2-mercaptoethanol.

Media can include RPMI 1640, AIM-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of immune cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2).

The medium used to culture the immune cells may include an agent that can co-stimulate the immune cells. For example, an agent that can stimulate CD3 is an antibody to CD3, and an agent that can stimulate CD28 is an antibody to CD28. This is because, as demonstrated by the data disclosed herein, a cell isolated by the methods disclosed herein can be expanded approximately 10 fold, 20 fold, 30 fold, 40 fold, 50 fold, 60 fold, 70 fold, 80 fold, 90 fold, 100 fold, 200 fold, 300 fold, 400 fold, 500 fold, 600 fold, 700 fold, 800 fold, 900 fold, 1000 fold, 2000 fold, 3000 fold, 4000 fold, 5000 fold, 6000 fold, 7000 fold, 8000 fold, 9000 fold, 10,000 fold, 100,000 fold, 1,000,000 fold, 10,000,000 fold, or greater. In one embodiment, the immune cells expand in the range of about 20 fold to about 50 fold, or more by culturing the electroporated population. In one embodiment, human T regulatory cells are expanded via anti-CD3 antibody coated KT64.86 artificial antigen presenting cells (aAPCs). Methods for expanding and activating immune cells can be found in U.S. Pat. Nos. 7,754,482, 8,722,400, and 9,555,105, contents of which are incorporated herein in their entirety.

D. Sources of Immune Cells

Prior to expansion, a source of immune cells is obtained from a subject for ex vivo manipulation. Sources of target cells for ex vivo manipulation may also include, e.g., autologous or heterologous donor blood, cord blood, or bone marrow. For example, the source of immune cells may be from the subject to be treated with the modified immune cells of the invention, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD 8+ naive T cell, central memory T cell, or effector memory T cell), a CD4+ T cell, a natural killer T cell (NKT cells), a regulatory T cell (Treg), a stem cell memory T cell, a lymphoid progenitor cell, a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation.

Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MATT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In certain aspects, the sample from which the immune cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in certain aspects, contain lymphocytes, including T cells, monocytes, granulocytes, B cells, other nucleated white blood cells, red blood cells, and/or platelets, and in certain aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some certain, a washing step is accomplished by tangential flow filtration (TFF) according to the manufacturer's instructions. In certain embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺-free, Mg²⁺-free PBS, PlasmaLyte A, or another saline solution with or without buffer. In some embodiments, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in certain aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the cells having bound the reagents are retained for further use, and/or negative selection, in which the cells having not bound to the antibody or binding partner are retained. In some examples, both fractions are retained for further use. In certain aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells. In certain exemplary embodiments, multiple rounds of separation steps are carried out, where the positively or negatively selected fraction from one step is subjected to another separation step, such as a subsequent positive or negative selection. In certain exemplary embodiments, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of tire T cell populations is enriched for or depleted of cells that are positive for (markeri−) or express high levels (marker^{high}) of one or more particular markers, such as surface markers, or that are negative for (marker−) or express relatively low levels (marker^{low}) of one or more markers. For example, in certain aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD127, and/or CD62L and/or depleted of (e.g., negatively selected for) cells that are positive for or express high surface levels of CD45RA. In some embodiments, cells are enriched for or depleted of cells positive or expressing high surface levels of CD122, CD95, CD25, CD27, and/or IL7-Ra (CD 127). In certain exemplary embodiments, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In certain aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in certain aspects is particularly robust in such sub-populations.

In some embodiments, combining TCM-enriched CD8+ T cells and CD4+ T cells further enhances efficacy. In some embodiments, memory T cells are present in both CD62L+ and CD62L− subsets of CD8+ peripheral blood lymphocytes. PBMC can be enriched for or depleted of CD62L−CD8+ and/or CD62L+CD8+ fractions, such as using anti-CD8 and anti-CD62L antibodies. In some embodiments, a CD4+ T cell population and/or a CD8+ T population is enriched for central memory (TCM) cells. In some embodiments, the enrichment for central memory T (TCM) cells is based on positive or high surface expression of CD45RO, CD62L, CCR7, CD28, CDS, and/or CD 127; in certain aspects, it is based on negative selection for cells expressing or highly expressing CD45RA and/or granzyme B. In certain aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in certain aspects are carried out simultaneously and in other aspects are carried out sequentially, in either order. In some certain, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4+ cells are CD62L− and CD45RO. In one example, to enrich for CD4+ cells by negative selection, a monoclonal antibody cocktail typically includes antibodies to CD14, CD20, CD11b, CD16, HLA-DR, and CDS. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In certain aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/ml.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CDS, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. An exemplary method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CDS.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in anon-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In some embodiments, the immune cell is obtained from a blood sample, a whole blood sample, a peripheral blood mononuclear cell (PBMC) sample, or an apheresis sample. In some examples, cells from the circulating blood of a subject are obtained, e.g., by apheresis or leukapheresis. In one embodiment, immune cells are obtained from the circulating blood of an individual are obtained by apheresis or leukapheresis. The apheresis product typically contains lymphocytes, including T cells, monocytes, granulocytes, B cells, other nucleated white blood cells, red blood cells, and platelets. In some embodiments, the apheresis sample is a cryopreserved sample. In some embodiments, the apheresis sample is a fresh sample. In some embodiments, the immune cell is obtained from a human subject.

### VII. Compositions

In one aspect the present invention provides a composition comprising the modified immune cell described herein, or a population of modified immune cells obtained from any of the methods described herein. In some embodiment, the compositions of the present invention may comprise a modified unstimulated T cell or a modified stimulated T cell as described herein. In some embodiments, the composition may include a pharmaceutical composition. In some embodiments, the composition may include a pharmaceutical composition and further comprises one or more pharmaceutically or physiologically acceptably carriers, diluents, adjuvants, or excipients. Such compositions may comprise buffers such as neutral buffered saline, phosphate buffered saline and the like; carbohydrates such as glucose, mannose, sucrose, or dextrans, mannitol; proteins; polypeptides or amino acids such as glycine, antioxidants; chelating agents such as EDTA or glutathione; adjuvants (e.g., aluminum hydroxide); and preservatives. Compositions of the present invention are preferably formulated for parenteral administration (e.g., intravenous administration). In some embodiments, a therapeutically effective amount of the pharmaceutical composition comprising the modified T cells may be administered to a subject in need thereof.

### VIII. Methods of Treatment

In one aspect, the present disclosure provides a method for adoptive cell transfer therapy comprising administering to a subject in need thereof a modified immune cell of the present invention. In some embodiments, disclosed herein is a method of treating a disease or a condition in a subject, which comprises administering to the subject a population of modified T cells described herein, e.g., a population of modified unstimulated T cells or a population of modified stimulated T cells described herein. In some embodiments, the invention includes a method of treating a disease or condition in a subject comprising administering to a subject in need thereof a composition comprising the modified immune cells described herein. In some embodiments, the method of treating a disease or condition in a subject comprises administering to a subject in need thereof a modified immune cell (e.g., T cell) comprising an insertion and/or deletion in one or more gene loci each encoding an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain) and an exogenous nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen. In some embodiments, the insertion and/or deletion is capable of downregulating gene expression of the one or more endogenous immune genes.

In some embodiments, the modified immune cell further comprises a dominant negative receptor, a switch receptor, a chemokine, a chemokine receptor, a cytokine, a cytokine receptor, IL-7, IL-7R, IL-15, IL-15R, IL-21, IL-18, CCL21, CCL19, or a combination thereof. In some instances, the disease is a cancer, optionally a solid tumor or a hematologic malignancy. In some instances, the modified unstimulated T cells or the modified stimulated T cells each expresses an antigen-binding domain that is specific for an antigen expressed by the cancer. In some embodiments, the method comprises administering to a subject in need thereof a modified immune cell (e.g., T cell) comprising a nucleic acid capable of downregulating gene expression, a TCR, a KIR, a CAR, a dominant negative receptor, and/or a switch receptor as described elsewhere herein. In some embodiments, the modified immune cell is a universal TCR redirected T cell (e.g. allogeneic T cell).

In some embodiments, the cancer is a solid tumor. Exemplary solid tumors include, but are not limited to, bladder cancer, bone cancer, brain cancer (e.g., glioma, glioblastoma, neuroblastoma), breast cancer, colorectal cancer, esophageal cancer, eye cancer, head and neck cancer, kidney cancer, lung cancer, melanoma, mesothelioma, ovarian cancer, pancreatic cancer, prostate cancer, or stomach cancer. In some instances, the solid tumor is brain cancer (e.g., glioma, glioblastoma, neuroblastoma), breast cancer, lung cancer, melanoma, mesothelioma, ovarian cancer, pancreatic cancer, or prostate cancer. In some instances, the solid tumor is a metastatic cancer. In some cases, the solid tumor is a relapsed or refractory solid tumor.

In some embodiments, the cancer is a hematologic malignancy. In some embodiments, the hematologic malignancy is a B-cell malignancy or a T-cell malignancy. In some embodiments, the hematologic malignancy is a lymphoma, a leukemia, or a myeloma. In some embodiments, the hematologic malignancy is a Hodgkin's lymphoma, or a non-Hodgkin's lymphoma. Exemplary hematologic malignancy include, but are not limited to, chronic lymphocytic leukemia (CLL), small lymphocytic lymphoma (SLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. In some instances, the hematologic malignancy is a metastatic hematologic malignancy. In some cases, the hematologic malignancy is a relapsed or refractory hematologic malignancy.

In some embodiments, the method of treating a disease further comprises administering to the subject an additional therapeutic agent or an additional therapy. In some cases, an additional therapeutic agent disclosed herein comprises a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.In some cases, the additional therapeutic agent comprises a first-line therapy. As used herein, “first-line therapy” comprises a primary treatment for a subject with a cancer. In some instances, the cancer is a primary cancer. In other instances, the cancer is a metastatic or recurrent cancer. In some cases, the first-line therapy comprises chemotherapy. In other cases, the first-line treatment comprises radiation therapy. A skilled artisan would readily understand that different first-line treatments may be applicable to different type of cancers. In some cases, the additional therapeutic agent comprises an immune checkpoint inhibitor. In some instances, the immune checkpoint inhibitor comprises an inhibitors such as an antibody or fragments (e.g., a monoclonal antibody, a human, humanized, or chimeric antibody) thereof, RNAi molecules, or small molecules to PD-1, PD-L1, CTLA4, PD-L2, LAG3, B7-H3, KIR, CD137, PS, TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. Exemplary checkpoint inhibitors include pembrolizumab, nivolumab, tremelimumab, or ipilimumab. In some embodiments, the additional therapy comprises radiation therapy.

In some embodiments, the additional therapy comprises surgery.

### IX. Kits and Articles of Manufacture

In some embodiments, a kit or article of manufacture described herein includes one or more populations of the modified T cells (e.g., modified unstimulated T cells or modified stimulated T cells). In some instances, the kit or article of manufacture described herein further include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment.

A kit typically includes labels listing contents and/or instructions for use, and package inserts with instructions for use. A set of instructions will also typically be included.

### IX. Definitions

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

It is also to be understood that the terminology used herein is for the purpose of describing particular embodiments only, and is not intended to be limiting.

The practice of the present disclosure will employ, unless otherwise indicated, conventional techniques of tissue culture, immunology, molecular biology, microbiology, cell biology and recombinant DNA, which are within the skill of the art. See e.g., Green and Sambrook eds. (2012) Molecular Cloning: A Laboratory Manual, 4th edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6th edition; Gait ed. (1984) Oligonucleotide Synthesis; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2nd edition; Guisan ed. (2006) Immobilization of Enzymes and Cells; Perbal (1988) A Practical Guide to Molecular Cloning, 2nd edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4th edition; and Herzenberg et al. eds (1996) Weir's Handbook of Experimental Immunology, 5th edition.

As used herein, the singular forms “a”, “an,” and “the” include plural referents unless the context clearly indicates otherwise. For example, the term “a cell” includes a plurality of cells, including mixtures thereof, and means one cell or more than one cell

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) (±) 20%, 15%, 10%, 5%, 3%, 2%, or 1%. Preferably ±5%, more preferably ±1%, and still more preferably ±0.1% from the specified value, as such variations are appropriate to perform the disclosed methods.

As used herein, the term “Activation” refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

“Allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

As used herein, the term “allogeneic T cell target” or “allogeneic T-cell” refers to a protein that mediates or contributes to a host versus graft response, mediates or contributes to a graft versus host response, or is a target for an immunosuppressant; and the gene encoding said molecule and its associated regulatory elements (e.g., promoters). It will be understood that the term allogeneic T cell target refers to the gene (and its associated regulatory elements) encoding an allogeneic T cell target protein when it is used in connection with a target sequence or gRNA molecule. Without being bound by theory, inhibition or elimination of one or more allogeneic T cell targets (e.g., by the methods and compositions disclosed herein) may improve the efficacy, survival, function and/or viability of an allogeneic cell. In some embodiments, the efficacy, survival, function and/or viability of an allogeneic cell is improved by reducing or eliminating undesirable immunogenicity (such as a host versus graft response or a graft versus host response). In some embodiments, the protein that mediates or contributes to a graft versus host response or host versus graft response is one or more components of the T cell receptor complex. In some embodiments, the component of the T cell receptor complex is the T cell receptor alpha, the constant domain of the TCR alpha (TRAC; TCRα). In some embodiments, the component of the T cell receptor is the T cell receptor beta chain (TRBC; TCR-β), for example the constant domain 1 (TRBC1) or constant domain 2 (TRBC2) of the TCR beta. In some embodiments, the component of the T cell receptor is the T cell receptor delta chain (CD3δ), the T cell receptor epsilon chain (CD3ε), the T cell receptor zeta chain (CD3ζ; CD247), and/or the T cell receptor gamma chain (CD3γ). In some embodiments, where the protein encoded by the allogeneic T cell target is a component of the TCR signaling complex, the gene encoding the allogeneic T cell target may be, for example, TRAC, TRBC1, TRBC2, CD3δ, CD3ε, CD3γ, or CD3ζ (CD247), or any combination thereof.

As used herein, the term “antibody” refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)2, as well as single chain antibodies (scFv) and humanized antibodies. In some embodiments, antibody refers to such assemblies (e.g., intact antibody molecules, immunoadhesins, or variants thereof) which have significant known specific immunoreactive activity to an antigen of interest (e.g. a tumor associated antigen). Antibodies and immunoglobulins comprise light and heavy chains, with or without an interchain covalent linkage between them. Basic immunoglobulin structures in vertebrate systems are relatively well understood.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

As used herein, the term “antibody heavy chain,” as used herein, refers to the larger of the two types of polypeptide chains present in all antibody molecules in their naturally occurring conformations.

As used herein, an “antibody light chain,” refers to the smaller of the two types of polypeptide chains present in all antibody molecules in their naturally occurring conformations. α and β light chains refer to the two major antibody light chain isotypes.

As used herein, the term “synthetic antibody” means an antibody, which is generated using recombinant DNA technology, such as, for example, an antibody expressed by a bacteriophage as described herein. The term should also be construed to mean an antibody, which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The antigen binding domain of (e.g., a chimeric antigen receptor) includes antibody variants. As used herein, the term “antibody variant” includes synthetic and engineered forms of antibodies which are altered such that they are not naturally occurring, e.g., antibodies that comprise at least two heavy chain portions but not two complete heavy chains (such as, domain deleted antibodies or minibodies); multi-specific forms of antibodies (e.g., bi-specific, tri-specific, etc.) altered to bind to two or more different antigens or to different epitopes on a single antigen); heavy chain molecules joined to scFv molecules and the like. In addition, the term “antibody variant” includes multivalent forms of antibodies (e.g., trivalent, tetravalent, etc., antibodies that bind to three, four or more copies of the same antigen.

As used herein, the term “antigen” or “Ag” is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequence or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit a desired immune response. Moreover, the skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “anti-tumor effect” refers to a biological effect which can be manifested by a decrease in tumor volume, a decrease in the number of tumor cells, a decrease in the number of metastases, an increase in life expectancy, or amelioration of various physiological symptoms associated with the cancerous condition. In some embodiments, an “anti-tumor effect” can also be manifested by the ability of the peptides, polynucleotides, cells and antibodies of the invention in prevention of the occurrence of tumor in the first place.

As used herein, the term “auto-antigen” means, in accordance with the present invention, any self-antigen, which is recognized by the immune system as being foreign. In some embodiments, Auto-antigens comprise, but are not limited to, cellular proteins, phosphoproteins, cellular surface proteins, cellular lipids, nucleic acids, glycoproteins, including cell surface receptors.

As used herein, the term “autoimmune disease” as used herein is defined as a disorder that results from an autoimmune response. An autoimmune disease is the result of an inappropriate and excessive response to a self-antigen. Examples of autoimmune diseases include but are not limited to, Addision's disease, alopecia areata, ankylosing spondylitis, autoimmune hepatitis, autoimmune parotitis, cancer, Crohn's disease, diabetes (Type I), dystrophic epidermolysis bullosa, epididymitis, glomerulonephritis, Graves' disease, Guillain-Barr syndrome, Hashimoto's disease, hemolytic anemia, systemic lupus erythematosus, multiple sclerosis, myasthenia gravis, pemphigus vulgaris, psoriasis, rheumatic fever, rheumatoid arthritis, sarcoidosis, scleroderma, Sjogren's syndrome, spondyloarthropathies, thyroiditis, vasculitis, vitiligo, myxedema, pernicious anemia, ulcerative colitis, among others.

As used herein, the term “autologous” is meant to refer to any material derived from the same individual into whom the material may later be re-introduced.

As used herein, the term “cancer” refers to a disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, metastatic castrate-resistant prostate cancer, melanoma, synovial sarcoma, advanced TnMuc1 positive solid tumors, neuroblastoma, neuroendocrine tumors, and the like. In certain embodiments, the cancer is medullary thyroid carcinoma. In certain embodiments, the cancer is prostate cancer. In certain embodiments, the cancer is mesothelioma or a mesothelin expressing cancer. In some embodiments, the cancer is metastatic castrate-resistant prostate cancer. The terms “cancer” and “tumor” are used interchangeably herein, and both terms encompass solid and liquid tumors, diffuse or circulating tumors. In some embodiments, the cancer or tumor includes premalignant, as well as malignant cancers and tumors.

As used herein, the term “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells (e.g., a lineage marker such as CD19 on B cells). In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described. For example, TCR-like antibody can be identified from screening a library, such as a human scFv phage displayed library.

As used herein, the term “cancer-supporting antigen” or “tumor-supporting antigen” interchangeably refers to a molecule (typically a protein, carbohydrate or lipid) that is expressed on the surface of a cell that is, itself, not cancerous, but supports the cancer cells by promoting their growth or survival (e.g., resistance to immune cells). Exemplary cells of this type include stromal cells and myeloid-derived suppressor cells (MDSCs). The tumor-supporting antigen itself need not play a role in supporting the tumor cells so long as the antigen is present on a cell that supports cancer cells.

As used herein, the term “Cas,” “Cas molecule,” or “Cas molecule” refers to an enzyme from bacterial Type II CRISPR/Cas system responsible for DNA cleavage. Cas includes wild-type protein as well as functional and non-functional mutants thereof.

As used herein, the term “chimeric antigen receptor” or “CAR,” refers to an artificial T cell receptor that is engineered to be expressed on an immune effector cell or precursor cell thereof and specifically bind an antigen. CARs may be used in adoptive cell therapy with adoptive cell transfer. In some embodiments, adoptive cell transfer (or therapy) comprises removal of T cells from a patient, and modifying the T cells to express the receptors specific to a particular antigen. In some embodiments, the CAR has specificity to a selected target, for example, ROR1, mesothelin, c-Met, PSMA, PSCA, Folate receptor alpha, Folate receptor beta, EGFR, EGFRvIII, GPC2, GPC2, Mucin 1 (MUC1), Tn antigen ((Tn Ag) or (GalNAca-Ser/Thr)), TnMUC1, GDNF family receptor alpha-4 (GFRa4), fibroblast activation protein (FAP), or Interleukin-13 receptor subunit alpha-2 (IL-13Ra2 or CD213A2). In some embodiments, CARs may also comprise an intracellular activation domain, a transmembrane domain and an extracellular domain comprising a tumor associated antigen binding region. In some aspects, CARs comprise fusions of single-chain variable fragments (scFv) derived monoclonal antibodies, fused to CD3-zeta transmembrane and intracellular domain. The specificity of CAR designs may be derived from ligands of receptors (e.g., peptides). In some embodiments, a CAR can target cancers by redirecting the specificity of a T cell expressing the CAR specific for tumor associated antigens.

As used herein, the term “cleavage” refers to the breakage of covalent bonds, such as in the backbone of a nucleic acid molecule. Cleavage can be initiated by a variety of methods, including, but not limited to, enzymatic or chemical hydrolysis of a phosphodiester bond. Both single-stranded cleavage and double-stranded cleavage are possible. Double-stranded cleavage can occur as a result of two distinct single-stranded cleavage events. DNA cleavage can result in the production of either blunt ends or staggered ends. In certain embodiments, fusion polypeptides may be used for targeting cleaved double-stranded DNA.

As used herein, the term “complementary” as used in connection with nucleic acid, refers to the pairing of bases, A with T or U, and G with C. The term complementary refers to nucleic acid molecules that are completely complementary, that is, form A to T or U pairs and G to C pairs across the entire reference sequence, as well as molecules that are at least 80%, 85%, 90%, 95%, 99% complementary.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody can be replaced with other amino acid residues from the same side chain family and the altered antibody can be tested for the ability to bind antigens using the functional assays described herein.

As used herein, the term “Co-stimulatory ligand,” includes a molecule on an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD2, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

As used herein, a “co-stimulatory molecule” refers to the cognate binding partner on a T cell that specifically binds with a co-stimulatory ligand, thereby mediating a co-stimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA, a Toll ligand receptor, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR). In some embodiments, a co-stimulatory molecule includes OX40, CD27, CD2, CD28, ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

As used herein, the term “co-stimulatory signal” refers to a signal, which in combination with a primary signal, such as TCR/CD3 ligation, leads to T cell proliferation and/or upregulation or downregulation of key molecules. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, BAFFR, HVEM, ICAM-1, lymphocyte function-associated antigen-1 (LFA-1), CD2, CDS, CD7, CD287, LIGHT, NKG2C, NKG2D, SLAMF7, NKp80, NKp30, NKp44, NKp46, CD160, B7-H3, and a ligand that specifically binds with CD83, and the like.

As used herein, the term “CRISPR” refers to clustered regularly interspaced short palindromic repeats system. The term “CRISPR system,” “CRISPR/Cas,” “CRISPR/Cas system,” or “CRISPR” refers to DNA loci containing short repetitions of base sequences. Each repetition is followed by short segments of spacer DNA from previous exposures to a virus. Bacteria and archaea have evolved adaptive immune defenses termed CRISPR-CRISPR-associated (Cas) systems that use short RNA to direct degradation of foreign nucleic acids. In bacteria, the CRISPR system provides acquired immunity against invading foreign DNA via RNA-guided DNA cleavage. In the type II CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

In some embodiments, the term “CRISPR system,” “CRISPR/Cas,” “CRISPR/Cas system,” or “CRISPR” refers to a set of molecules comprising an RNA-guided nuclease or other effector molecule and a gRNA molecule that together are necessary and sufficient to direct and effect modification of nucleic acid at a target sequence by the RNA-guided nuclease or other effector molecule. In some embodiments, a CRISPR system comprises a gRNA and a Cas protein, e.g., a Cas3, Cas4, Cas8a, Cas8b, Cas9, Cas10, Cas10d, Cas12a, Cas12b, Cas12d, Cas12e, Cas12f, Cas12g, Cas12h, Cas12i, Cas13, Cas14, CasX, Cse1, Csy1, Csn2, Cpf1, C2c1, Csm2, Cmr5, Fok1, S. pyogenes Cas9 (spCas9), or Staphylococcus aureus Cas9 (saCas9) protein. Such systems comprising a Cas or modified Cas molecule are referred to herein as “Cas systems” or “CRISPR/Cas systems.” In some embodiments, the gRNA molecule and Cas molecule may be complexed, to form a ribonuclear protein (RNP) complex.

To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering and therapeutics.

As used herein, the term “crRNA” as the term is used in connection with a gRNA molecule, is a portion of the gRNA molecule that comprises a targeting domain and a region that interacts with a tracr to form a flagpole region.

As used herein, the term “CRISPRi” refers to a CRISPR system for sequence specific gene repression or inhibition of gene expression, such as at the transcriptional level.

As used herein, the term “Derived from” refers to a relationship between a first and a second molecule. It defines a structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, It does not mean that, to provide the intracellular signaling domain, one must start with a CD3zeta sequence and delete unwanted sequence, or impose mutations, to arrive at the intracellular signaling domain.

As used herein, the term “disease” refers to a state of health of an animal wherein the animal cannot maintain homeostasis, and wherein if the disease is not ameliorated then the animal's health continues to deteriorate. In contrast, the term “disorder” in an animal refers to a state of health in which the animal is able to maintain homeostasis, but in which the animal's state of health is less favorable than it would be in the absence of the disorder. Left untreated, a disorder does not necessarily cause a further decrease in the animal's state of health.

As used herein, “disease associated with expression of a tumor antigen” includes, but is not limited to, a disease associated with expression of a tumor antigen or condition associated with cells which express a tumor antigen including, but not limited to proliferative diseases such as a cancer or malignancy or a precancerous condition such as a myelodysplasia, a myelodysplastic syndrome or a preleukemia; or a noncancer related indication associated with cells, which express a tumor antigen. In some embodiments, a cancer associated with expression of a tumor antigen is a hematological cancer. In some embodiments, a cancer associated with expression of a tumor antigen is a solid cancer. Further diseases associated with expression of a tumor antigen include, but not limited to, atypical and/or non-classical cancers, malignancies, precancerous conditions or proliferative diseases associated with expression of a tumor antigen. Non-cancer related indications associated with expression of a tumor antigen include, but are not limited to, autoimmune disease, (e.g., lupus), inflammatory disorders (allergy and asthma) and transplantation. In some embodiments, the tumor antigen-expressing cells express, or at any time expressed, mRNA encoding the tumor antigen. In some embodiments, the tumor antigen-expressing cells produce the tumor antigen protein (e.g., wild-type or mutant), and the tumor antigen protein may be present at normal levels or reduced levels. In some embodiment, the tumor antigen-expressing cells produced detectable levels of a tumor antigen protein at one point, and subsequently produced substantially no detectable tumor antigen protein.

As used herein, the term “downregulation” refers to the decrease or elimination of gene expression of one or more genes.

As used herein, the term “Encoding” refers to the inherent property of specific sequences of nucleotides in a polynucleotide (such as a gene, a cDNA, or an mRNA), to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

“Effective amount” or “therapeutically effective amount” as used interchangeably herein, refer to an amount of a compound, formulation, material, pharmaceutical agent, or composition, as described herein effective to achieve a desired physiological, therapeutic, or prophylactic outcome in a subject in need thereof. Such results may include, but are not limited to an amount that when administered to a mammal, causes a detectable level of immune response compared to the immune response detected in the absence of the composition of the invention. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like. The effective amount may vary among subjects depending on the health and physical condition of the subject to be treated, the taxonomic group of the subjects to be treated, the formulation of the composition, assessment of the subject's medical condition, and other relevant factors.

As used herein, the term “endogenous” refers to any material from or produced inside an organism, cell, tissue or system.

As used herein, the term “Expand” as used herein refers to increasing in number, as in an increase in the number of immune cells (e.g. T cells). In some embodiments, the immune cells (e.g. T cells) that are expanded ex vivo increase in number relative to the number originally present in the culture. In another embodiment, the immune cells (e.g. T cells) that are expanded ex vivo increase in number relative to other cell types in the culture.

As used herein, the term “Expression” refers to the transcription and/or translation of a particular nucleotide sequence driven by a promoter.

As used herein, the term “Exogenous” refers to any material introduced from or produced outside an organism, cell, tissue or system.

As used herein, the term “Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “ex vivo,” refers to cells that have been removed from a living organism, (e.g., a human) and propagated outside the organism (e.g., in a culture dish, test tube, or bioreactor).

As used herein, the term “flagpole” used in connection with a gRNA molecule, refers to a portion of a gRNA where the crRNA and the tracr bind to, or hybridize to, one another.

As used herein, the term “guide RNA,” “guide RNA molecule,” “”gRNA molecule” or “gRNA” are used interchangeably, and refers to a set of nucleic acid molecules that promote the specific directing of a RNA-guided nuclease or other effector molecule (typically in complex with the gRNA molecule) to a target sequence. In some embodiments, said directing is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In some embodiments, a gRNA molecule consists of a single contiguous polynucleotide molecule, referred to herein as a “single guide RNA” or “sgRNA” and the like. In some embodiments, a gRNA molecule consists of a plurality, usually two, polynucleotide molecules, which are themselves capable of association, usually through hybridization, referred to herein as a “dual guide RNA” or “dgRNA,” and the like. gRNA molecules are described in more detail below, but generally include a targeting domain and a tracr. In some embodiments the targeting domain and tracr are disposed on a single polynucleotide. In other embodiments, the targeting domain and tracr are disposed on separate polynucleotides.

As used herein, the term “Homologous” refers to the subunit sequence identity between two polymeric molecules (e.g., between two nucleic acid molecules, such as, two DNA molecules or two RNA molecules), or between two polypeptide molecules. When a subunit position in both of the two molecules is occupied by the same monomeric subunit, then they are homologous at that position. For example, if a position in each of two DNA molecules is occupied by adenine, then the two DNA molecules are homologous. The homology between two sequences is a direct function of the number of matching or homologous positions. For example, if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

As used herein, the term “Humanized antibodies” refers to human forms of non-human (e.g., murine) antibodies, and are chimeric immunoglobulins, immunoglobulin chains or fragments thereof (such as Fv, Fab, Fab′, F(ab′)2 or other antigen-binding subsequences of antibodies), which contain minimal sequence derived from non-human immunoglobulin. For the most part, humanized antibodies are human immunoglobulins (recipient antibody) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies can comprise residues which are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. The humanized antibody optimally also will comprise at least a portion of an immunoglobulin constant region (Fc), typically that of a human immunoglobulin. For further details, see Jones et al., Nature 321: 522-525 (1986); Reichmann et al., Nature 332: 323-329 (1988); Presta, Curr. Op. Struct. Biol. 2: 593-596 (1992).

As used herein, the term “Fully human” refers to an immunoglobulin, such as an antibody, where the whole molecule is of human origin or consists of an amino acid sequence identical to a human form of the antibody.

As used herein, the term “Identity” refers to the subunit sequence identity between two polymeric molecules particularly between two amino acid molecules, such as, between two polypeptide molecules. When two amino acid sequences have the same residues at the same positions, then they are identical at that position. For example, if a position in each of two polypeptide molecules is occupied by an Arginine, then the two polypeptides are identical. The identity or extent to which two amino acid sequences have the same residues at the same positions in an alignment is often expressed as a percentage. The identity between two amino acid sequences is a direct function of the number of matching or identical positions. For example, if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, the term “immunoglobulin” or “Ig,” defines a class of proteins, which function as antibodies. Antibodies expressed by B cells are sometimes referred to as the BCR (B cell receptor) or antigen receptor. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function, but may serve as an antigen receptor. IgE is the immunoglobulin that mediates immediate hypersensitivity by causing release of mediators from mast cells and basophils upon exposure to allergen.

As used herein, the term “immune response” as used herein is defined as a cellular response to an antigen that occurs when lymphocytes identify antigenic molecules as foreign and induce the formation of antibodies and/or activate lymphocytes to remove the antigen.

As used herein, the term “Immune effector cell,” refers to a cell that is involved in an immune response, e.g., in the promotion of an immune effector response. Examples of immune effector cells include T cells (e.g., alpha/eta T cells and gamma/delta T cells), B cells, natural killer (NK) cells, natural killer T (NKT) cells, mast cells, and myeloic-derived phagocytes.

As used herein, the term “Immune effector function or immune effector response,” refers to a function or response that enhances or promotes an immune attack of a target cell. In some embodiment, an immune effector function or response refers to a property of a T or NK cell that promotes the killing or the inhibition of growth or proliferation, of a target cell. In the case of a T cell, primary stimulation and co-stimulation are examples of immune effector function or response.

As used herein, the term “inhibitory molecule” refers to a molecule, which when activated, causes or contributes to an inhibition of cell survival, activation, proliferation and/or function; and the gene encoding said molecule and its associated regulatory elements (e.g., promoters). In some embodiments, an inhibitory molecule is a molecule expressed on an immune effector cell (e.g., on a T cell). Non-limiting examples of inhibitory molecules are PD-1, PD-L1, PD-L2, CTLA4, TIM3, LAG3, CEACAM (e.g., CEACAM-1, CEACAM-3 and/or CEACAM-5), VISTA, TGFβIIR, VSIG3, VSIG 8, BTLA, TIGIT, LAIR1, CD160, 2B4, CD80, CD86, B7-H3 (CD276), B7-H4 (VTCN1), HVEM (TNFRSF14 or CD107), KIR, A2aR, MHC class I, MHC class II, GAL9, adenosine, and TGF beta. It will be understood that the term inhibitory molecule refers to the gene (and its associated regulatory elements) encoding an inhibitory molecule protein when it is used in connection with a target sequence or gRNA molecule. In some embodiments, gene encoding the inhibitory molecule is BTLA, PD-1, TIM-3, VSIG3, VSIG8, CTLA4, or TGFβIIR. In some embodiments, the gene encoding the inhibitory molecule is VSIG3. In some embodiments, the gene encoding the inhibitory molecule is PD-1. In some embodiments, the gene encoding the inhibitory molecule is TGFβIIR.

As used herein, the term “induced pluripotent stem cell” or “iPS cell” refers to a pluripotent stem cell that is generated from adult cells, such immune cells (i.e. T cells). The expression of reprogramming factors, such as Klf4, Oct3/4 and Sox2, in adult cells convert the cells into pluripotent cells capable of propagation and differentiation into multiple cell types.

As used herein, the term “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

As used herein, the term “Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the term “Knockout” refers to the ablation of gene expression of one or more genes.

As used herein, the term “Lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

As used herein, the term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n, where n is a positive integer equal to or greater than 1. For example, n=1, n=2, n=3, n=4, n=5 and n=6, n=7, n=8, n=9 and n=10 (SEQ ID NO:48). In one embodiment, the flexible polypeptide linkers include, but are not limited to, (Gly4 Ser)4 or (Gly4 Ser)3. In another embodiment, the linkers include multiple repeats of (Gly2Ser), (GlySer) or (Gly3Ser).

As used herein, the term “Modified” means a changed state or structure of a molecule or cell of the invention. Molecules may be modified in many ways, including chemically, structurally, and functionally. Cells may be modified through the introduction of nucleic acids.

As used herein, the term “Modulating,” means mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

As used herein, the term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

As used herein, the term “overexpressed” tumor antigen or “overexpression” of a tumor antigen is intended to indicate an abnormal level of expression of a tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

As used herein, the term “Parenteral” administration of an immunogenic composition includes, e.g., subcutaneous (s.c.), intravenous (i.v.), intramuscular (i.m.), or intrasternal injection, or infusion techniques.

As used herein, the term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “promoter” is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

As used herein, the term “Constitutive promoter” is a nucleotide sequence which, when operably linked with a polynucleotide which encodes or specifies a gene product, causes the gene product to be produced in a cell under most or all physiological conditions of the cell.

As used herein, the term “inducible promoter” is a nucleotide sequence which, when operably linked with a polynucleotide which encodes or specifies a gene product, causes the gene product to be produced in a cell substantially only when an inducer which corresponds to the promoter is present in the cell.

As used herein, the term “tissue-specific promoter” is a nucleotide sequence which, when operably linked with a polynucleotide encodes or specified by a gene, causes the gene product to be produced in a cell substantially only if the cell is a cell of the tissue type corresponding to the promoter.

As used herein, the term “Sendai virus” refers to a genus of the Paramyxoviridae family. Sendai viruses are negative, single stranded RNA viruses that do not integrate into the host genome or alter the genetic information of the host cell. Sendai viruses have an exceptionally broad host range and are not pathogenic to humans. Used as a recombinant viral vector, Sendai viruses are capable of transient but strong gene expression.

As used herein, the term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell.

As used herein, the term “Single chain antibodies” refer to antibodies formed by recombinant DNA techniques in which immunoglobulin heavy and light chain fragments are linked to the Fv region via an engineered span of amino acids. Various methods of generating single chain antibodies are known, including those described in U.S. Pat. No. 4,694,778; Bird, Science 242:423-442 (1988); Huston et al., Proc. Natl. Acad. Sci. USA 85:5879-5883 (1988); Ward et al., Nature 334:54454 (1989); Skerra et al., Science 242:1038-1041 (1988).

As used herein, the term “specificity” refers to the ability to specifically bind (e.g., immunoreact with) a given target antigen (e.g., a human target antigen). A chimeric antigen receptor may be monospecific and contain one or more binding sites which specifically bind a target or a chimeric antigen receptor may be multi-specific and contain two or more binding sites which specifically bind the same or different targets. In certain embodiments, a chimeric antigen receptor is specific for two different (e.g., non-overlapping) portions of the same target. In certain embodiments, a chimeric antigen receptor is specific for more than one target.

As used herein, the term “specifically binds,” with respect to an antibody, means an antibody or binding fragment thereof (e.g., scFv) which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, a chimeric antigen receptor, or a peptide with a second chemical species, to mean that the interaction is dependent upon the presence of a particular structure (e.g., an antigenic determinant or epitope) on the chemical species; for example, a chimeric antigen receptor recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

As used herein, the term “Stimulation,” means a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, clonal expansion, and differentiation into distinct subsets.

As used herein, the term “Stimulatory molecule” means a molecule on a T cell that specifically binds with a cognate stimulatory ligand present on an antigen presenting cell.

As used herein, the term “Stimulatory ligand” means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, the terms “subject” and “patient” are used interchangeably. As used herein, a subject is can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets. Any living organism in which an immune response can be elicited may be a subject or patient. In certain exemplary embodiments, a subject is a human.

As used herein, the term “Substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

As used herein, the term “target site” or “target sequence” refers to a genomic nucleic acid sequence that defines a portion of a nucleic acid to which a binding molecule may specifically bind under conditions sufficient for binding to occur.

As used herein, the term “targeting domain” used in connection with a gRNA, refers to a portion of the gRNA molecule that recognizes, or is complementary to, a target sequence. For example, a target sequence within the nucleic acid of a cell (e.g., within a gene).

As used herein, the term “target sequence” refers to a sequence of nucleic acids complimentary, for example fully complementary, to a gRNA targeting domain. In some embodiments, the target sequence is disposed on genomic DNA. In some embodiment the target sequence is adjacent to (either on the same strand or on the complementary strand of DNA) a protospacer adjacent motif (PAM) sequence recognized by a protein having nuclease or other effector activity, e.g., a PAM sequence recognized by Cas9. In some embodiments, the target sequence is a target sequence of an allogeneic T cell target. In some embodiments, the target sequence is a target sequence of an inhibitory molecule. In some embodiments, the target sequence is a target sequence of a downstream effector of an inhibitory molecule.

As used herein, the term “T cell receptor” or “TCR” refers to a complex of membrane proteins that participate in the activation of T cells in response to the presentation of antigen. The TCR is responsible for recognizing antigens bound to major histocompatibility complex molecules. TCR is composed of a heterodimer of an alpha (a) and beta (β) chain, coupled to three dimeric modules CD3δ/CD3ε, CD3γ/CD3ε, and CD3ζ/CD3ζ. In some cells the TCR consists of gamma and delta (γ/δ) chains (CD3γ/CD3ε). In some embodiments, TCRs may exist in alpha/beta and gamma/delta forms, which are structurally similar but have distinct anatomical locations and functions. Each chain is composed of two extracellular domains, a variable and constant domain. In some embodiments, the TCR may be modified on any cell comprising a TCR, including, for example, a helper T cell, a cytotoxic T cell, a memory T cell, regulatory T cell, natural killer T cell, and gamma delta T cell.

The term “therapeutic” as used herein means a treatment and/or prophylaxis. A therapeutic effect is obtained by suppression, remission, or eradication of a disease state.

As used herein, the term “therapy” refers to any protocol, method and/or agent (e.g., a CAR-T) that can be used in the prevention, management, treatment and/or amelioration of a disease or a symptom related thereto. In some embodiments, the terms “therapies” and “therapy” refer to a biological therapy (e.g., adoptive cell therapy), supportive therapy (e.g., lymphodepleting therapy), and/or other therapies useful in the prevention, management, treatment and/or amelioration of a disease or a symptom related thereto, known to one of skill in the art such as medical personnel.

As used herein, the term “tracr” used in connection with a gRNA molecule, refers to a portion of the gRNA that binds to a nuclease or other effector molecule. In some embodiments, the tracr comprises nucleic acid sequence that binds specifically to Cas9. In some embodiments, the tracr comprises nucleic acid sequence that forms part of the flagpole. As used herein, the term “transfected” or “transformed” or “transduced” refers to a process by which an exogenous nucleic acid is transferred or introduced into a host cell. A “transfected” or “transformed” or “transduced” cell is one which has been transfected, transformed or transduced with an exogenous nucleic acid. The cell includes a primary subject cell and its progeny.

As used herein, the terms “treat,” “treatment” and “treating” refer to the reduction or amelioration of the progression, severity, frequency and/or duration of a disease or a symptom related thereto, resulting from the administration of one or more therapies (including, but not limited to, a CAR-T therapy directed to the treatment of solid tumors). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

As used herein, the term “Under transcriptional control” or “Operatively linked” as used herein means that the promoter is in the correct location and orientation in relation to a polynucleotide to control the initiation of transcription by RNA polymerase and expression of the polynucleotide.

As used herein, the term “Vector” is a composition of matter that comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

As used herein, the term “Xenogeneic” refers to a graft derived from an animal of a different species.

As used herein, the term “complete media” and “complete medium” refers to a cell culture media which are optimized for immune cell growth (e.g., T cell growth). In some instances, a complete media comprises proteins, inorganic salts, trace elements, vitamins, amino acids, lipids, carbohydrates, cytokines, and/or growth factors, in which the ratio of each components has been optimized for cell growth. Exemplary proteins include albumin, transferrin, fibronectin, and insulin. Exemplary carbohydrates include glucose. Exemplary inorganic salts incoude sodium, potassium, and calcium ions. Exemplary trace elements include zinc, copper, selenium, and tricarboxylic acid. Exemplary amino acids include essential amino acids such as L-glutamine (e.g., alanyl-1-glutamine or glycyl-1-glutamine); or non-essential amino acids (NEAA) such as glycine, L-alanine, L-asparagine, L-aspartic acid, L-glutamic acid, L-proline, and/or L-serine. In some embodiments, the complete media also comprises one or more of sodium bicarbonate (NaHCO3), HEPES (4-(2-hydroxyethyl)-1-piperazine ethanesulfonic acid), phenol red, antibiotics, and/or β-mercaptoethanol. In some instances, the complete media is a serum-free media. In some instances, the complete media is a xeno-free media.

As used herein, the term “chemically defined media” refers to a cell culture media in which the compositions and concentrations of all components are known. It differs from a complete media in that the complete media may contain components, e.g., animal-derived components, in which the composition and/or concentration are not known.

In some instances, a “xeno-free” media does not contain any animal-derived (non-human) component. In some instances, a xeno-free media contains one or more human-derived components such as human serum, growth factors, and insulin.

In some embodiments, a “serum-free” media does not contain serum or plasma but may contain components derived from serum or plasma. In some instances, the “serum-free” media contains animal-derived components such as bovine serum albumin (BSA).

In some embodiment, a “minimum” media comprises the minimal necessities for growth of a target cell. In some instances, the minimum media contains inorganic salts, carbon source, and water. In some instances, supplements, cytokines, and/or proteins such as albumin (e.g., HSA) are added to the minimum media. As used herein, supplements comprise trace elements, vitamins, amino acids, lipids, carbohydrates, cytokines, growth factors, or a combination thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

These examples are provided for illustrative purposes only and not to limit the scope of the claims provided herein.

### Example 1

This example provides materials and methods used to make the novel allogeneic CART cells of the present disclosure.

Primary Human T cell Expansions and Cell Cryopreservation. T cells were incubated at 1×10⁶ cells/mL in media and activated using Dynabeads® (ThermoFisher) at the beginning of the culture. Cells were counted every other day using a NC200™ Automated Cell Counter (Chemometec). In addition, cell viability and cell size were determined every other day. Cells were fed with fresh T-cell media and resuspended at 5×10⁵ cells/mL. On day 9-11, cells were harvested and cryopreserved in freezing media containing 5% DMSO.

Polychromatic Flow Cytometry Analysis. Cells were washed and resuspended in FACS buffer (phosphate-buffered saline containing 5% fetal calf serum). A master mix of antibodies conjugated to specific fluorophores was added to the cells. Cells were washed and resuspended in FACS buffer and analyzed using a MACSQuant® cytometer (Miltenyi). The antibody panel used included anti-CD3-VioBlue, anti-TCRab-APC, anti-B2M-PE-Vio770, anti-HLA-DR-VioGreen, anti-HLAI-ABC-FITC, anti-CAR-PE, and 7AAD for viability assessment. FlowJo® software (BD Biosciences) was used for data analysis.

Gene Editing of Primary Human T cells. Ribonucleoproteins (RNP) in the form of nuclease conjugated with the respective guide RNA for each target was transfected into human T cells. Cells were cultured in special T-cell media containing 5% human serum, IL-7, IL-15, and glutamine for 5-8 days. At the end of the cultures, polychromatic flow cytometry was performed to simultaneously determine the level of gene editing efficiency for each one of the gene targets.

Real-Time Tumor Cytotoxicity Assay. CAR T-cell cytotoxic capacity was assessed using the xCelligence™ Real-Time Cell Analysis instrument (Agilent). Briefly, CAR T cells were co-cultured in microtiter plates with tumor target cells at different effector-to-target ratios (e.g. 1:10, 1:5, 1:2.5, 1:1) for several days. Tumor cell death was determined continuously as the change in cellular impedance measured for each condition. Data was captured using the RTCA® software (Agilent).

Gene Editing Efficiency by T7E1 Endonuclease Assay. To determine gene editing efficiency at the molecular level we used the T7E1 endonuclease assay tailored to each particular gene target. Total genomic DNA was isolated from the T cells and stored at minus 80° C. or used directly for the assay. PCR reactions using primers spanning the cut site for each target were performed. PCR products were analyzed by gel electrophoresis to validate amplicon size and quantity. PCR amplicons were then digested using the T7E1 nuclease and analyzed using a 2100 High-Resolution Automated Electrophoresis BioAnalyzer® instrument (Agilent). The 2100 Expert® Software was used to determine gene editing efficiency.

Mixed-Leukocyte Reactions. Co-cultures of allogeneic CAR T cells with T cells from an unrelated donor (labeled as “2^{nd} donor”) were set up at different ratios and followed for 16 days. Cell death was determined by flow cytometry using specific markers to differentiate and track each particular cell type (i.e. allogeneic CART cells v. “2^{nd} donor's” T cells). 123count™ eBeads were added prior to flow cytometry to determine the absolute number of cells, and 7AAD dye was used to assess cell viability.

### Example 2

The purpose of this example was to detail a triple knockout strategy.

Table 2 below illustrates the novel allogeneic CART cell platform comprising a triple knockout strategy (3-Gene knockout combination) of the present disclosure. The unique approach involves targeting one or more genes of the TCR modules (CD3δ, CD3ε, and CD3γ), one or more genes of the HLA-I, and one or more gene of HLA-II.

TABLE 2
CRISPR Combinations

| | TCR | HLA-I | HLA-II |
|---|---|---|---|
| 1 | TRAC | B2M | C2TA |
| 2 | CD3ε | B2M | C2TA |
| 3 | CD3ε | B2M | RFX5 |
| 4 | CD3ε | B2M | RFXAP |
| 5 | CD3ε | B2M | RFXANK |
| 6 | CD3ε | B2M | HLA-DM |
| 7 | CD3ε | TAP1 | RFX5 |
| 8 | CD3ε | TAP1 | RFXANK |
| 9 | CD3ε | TAP1 | RFXAP |
| 10 | CD3ε | TAP1 | HLA-DM |
| 11 | CD3ε | NLRC5 | RFX5 |
| 12 | CD3ε | NLRC5 | RFXANK |
| 13 | CD3ε | NLRC5 | RFXAP |
| 14 | CD3ε | NLRC5 | HLA-DM |
| 15 | CD3ε | TAP2 | RFX5 |
| 16 | CD3ε | TAP2 | RFXANK |
| 17 | CD3ε | TAP2 | RFXAP |
| 18 | CD3ε | TAP2 | HLA-DM |
| 19 | CD3δ | B2M | C2TA |
| 20 | CD3δ | B2M | RFX5 |
| 21 | CD3δ | B2M | RFXAP |
| 22 | CD3δ | B2M | RFXANK |
| 23 | CD3δ | B2M | HLA-DM |
| 24 | CD3δ | TAP1 | RFX5 |
| 25 | CD3δ | TAP1 | RFXANK |
| 26 | CD3δ | TAP1 | RFXAP |
| 27 | CD3δ | TAP1 | HLA-DM |
| 28 | CD3δ | NLRC5 | RFX5 |
| 29 | CD3δ | NLRC5 | RFXANK |
| 30 | CD3δ | NLRC5 | RFXAP |
| 31 | CD3δ | NLRC5 | HLA-DM |
| 32 | CD3δ | TAP2 | RFX5 |
| 33 | CD3δ | TAP2 | RFXANK |
| 34 | CD3δ | TAP2 | RFXAP |
| 35 | CD3δ | TAP2 | HLA-DM |
| 36 | CD3γ | B2M | C2TA |
| 37 | CD3γ | B2M | RFX5 |
| 38 | CD3γ | B2M | RFXAP |
| 39 | CD3γ | B2M | RFXANK |
| 40 | CD3γ | B2M | HLA-DM |
| 41 | CD3γ | TAP1 | RFX5 |
| 42 | CD3γ | TAP1 | RFXANK |
| 43 | CD3γ | TAP1 | RFXAP |
| 44 | CD3γ | TAP1 | HLA-DM |
| 45 | CD3γ | NLRC5 | RFX5 |
| 46 | CD3γ | NLRC5 | RFXANK |
| 47 | CD3γ | NLRC5 | RFXAP |
| 48 | CD3γ | NLRC5 | HLA-DM |
| 49 | CD3γ | TAP2 | RFX5 |
| 50 | CD3γ | TAP2 | RFXANK |
| 51 | CD3γ | TAP2 | RFXAP |
| 52 | CD3γ | TAP2 | HLA-DM |

The strategy of knocking out multiple genes is expected to produce an improved allogenic T cell product.

### Example 3

The purpose of this example was to prepare various constructs as described herein, with at least one knockout gene.

FIG. 9 illustrates a novel allogeneic CART strategy of the present disclosure involving the knockout (KO) of alternative T cell receptor subunits (CD3δ, CD3γ, and CD3ε) and additional critical genes in the antigen processing and presentation pathways. In particular, 15 gene targets were selected and each was tested with multiple guide RNAs (gRNAs) using the CRISPR-associated (Cas) (CRISPR-Cas) endonuclease system. Exemplary gRNAs used to target the 15 genes are disclosed in Table 3.

TABLE 3
Exemplary gRNAs

| SEQ ID | | gRNA Sequence |
|---|---|---|
| NO | Gene Targets | (spacer) |
| 52 | CD3epsilon | AGATCCAGGATACTGAGGGCA |
| 53 | CD3delta | TCTCTGGCCTGGTACTGGCTA |
| 54 | CD3gamma | GCTTCTGCATCACAAGTCAGA |
| 55 | B2M | TATCTCTTGTACTACACTGA |
| 56 | TAP1 | GCTCTTGGAGCCAACCGTTG |
| 57 | TAP2 | CTTCCTCAAGGGCTGCCAGGA |
| 58 | TAPBP_gRNA1 | CCTACATGCCCCCCACCTCC |
| 59 | TAPBP_gRNA2 | CGCTCGCATCCTCCACGAAC |
| 60 | NLRC5 | GTGAGCAGCCTCACAAGACAG |
| 61 | C2TA | CCTTGGGGCTCTGACAGGTA |
| 62 | HLA-DMA | CCAGAACACTCGGGTGCCTCG |
| 63 | RFX5_gRNA1 | CAAGGCCGTGCAGAACAAAGT |
| 64 | RFX5_gRNA2 | TTCTGCACGGCCTTGGAAATG |
| 65 | RFXANK | CCTGCACCCCTGAGCCTGTGA |
| 66 | RFXAP | GAGGATCTAGAGGACGAGGAG |
| 67 | Ii Chain_gRNA1 | CATCCTGGTGACTCTGCTCCT |
| 68 | Ii Chain_gRNA2 | TCCAGCCGGCCCTGCTGCTGG |

### Example 4

The purpose of this example was to evaluate the efficiency of knockout of TCR α/β using different constructs focused on the targeted disruption of CD3δ (FIG. 2A), CD3ε (FIG. 2B), and CD3γ (FIG. 2C).

The percentage of TCR-α and TCR-β chains disruption efficiency on human T cells was measured by flow cytometry following the targeted disruption of CD3δ (FIG. 2A), CD3ε (FIG. 2B), and CD3γ (FIG. 2C) genes using a CRISPR/Cas system. FIG. 2A shows the results following disruption of CD3δ using four different guide RNAs: gRNA1, gRNA2, gRNA3, and gRNA4. Disruption of CD3δ using gRNA1 and gRNA3 guide RNAs in the CRISPR/Cas system resulted in 100% KO efficiency of TCR α/β, while use of gRNA2 and gRNA4 in the CRISPR/Cas system resulted in greater than about 90% KO efficiency of TCR α/β. Thus, use of gRNA1 and gRNA3 are preferred in a CRISPR/Cas system for disrupting CD3δ.

FIG. 2B shows the results following the targeted disruption of CD3ε using five different guide RNAs: gRNA1, gRNA2, gRNA3, gRNA4, and gRNA5 Disruption of CD3ε using gRNA4 and gRNA5 guide RNAs in the CRISPR/Cas system resulted in 100% KO efficiency of TCR α/β, while use of gRNA1 resulted in only about a 50% KO efficiency of TCR α/β, and finally use of guide gRNA2 and gRNA3 in the CRISPR/Cas system resulted in greater than about 90% KO efficiency of TCR α/β. Thus, use of use of gRNA4 and gRNA5 guide RNAs are preferred in a CRISPR/Cas system for disrupting CD3ε.

FIG. 2C shows the results following the targeted disruption of CD3γ using five different guide RNAs: gRNA1, gRNA2, gRNA3, gRNA4, and gRNA5 Disruption of CD3ε using gRNA4 and guide RNAs in the CRISPR/Cas system resulted in 100% KO efficiency of TCR α/β, while use of gRNA5 resulted in a greater than about 95% KO efficiency of TCR α/β, and finally use of gRNA1, gRNA2 and gRNA3 guide RNAs in the CRISPR/Cas system resulted in less favorable KO efficiency of TCR α/β. Thus, use of use of gRNA4 guide RNA are preferred in a CRISPR/Cas system for disrupting CD3γ.

### Example 5

The purpose of this example was to evaluate the expansion of different constructs of allogenic CART-cells over a 10 day period.

The different constructs tested included allogeneic CART cells comprising: (1) TRAC knockout (ALLO (TRAC KO) on FIG. 3) (e.g., the knockout used prior to the present disclosure), (2) CD3δ knockout (ALLO (D1 KO) on FIG. 3), (3) CD3γ knockout (ALLO (G4 KO) on FIG. 3), and (4) CD3ε knockout (ALLO (E4 KO) on FIG. 3). The percentage of the cell population doubling is shown on the Y axis while the number of days is shown on the X axis. The results detailed in FIG. 3.

FIG. 3 shows a graph illustrating the expansion of allogeneic CART-cells generated using the strategy of FIG. 1, and illustrates the number of population doublings over a ten-day period. The tested allogeneic CART cells are engineered T cells comprising TRAC knockout (TRAC KO), CD3δ knockout (D1 KO), CD3γ knockout (G4 KO), and CD3ε knockout (E4 KO).

### Example 6

This example evaluated several different knockout constructs to compare surface expression of the TCR-α/β chain.

FIGS. 4A and 4B show flow cytometry results comparing CRISPR-mediated downregulation of TCR-α chain (TRAC) knockout (e.g., the construct used prior to the present disclosure), CD3δ knockout (D1 KO), CD3γ knockout (G4 KO), and CD3ε knockout (E4 KO). FIG. 4A shows that CD3ε knockout (E4 KO) is a better target for T cell receptor knockout, as measured by surface expression of the TCR-α/β chain. FIG. 4B shows that allogeneic CART cells comprising CD3ε knockout (E4 KO) had higher transduction efficiency and were functionally better than CART cells comprising, for example CD3γ or CD3δ knockout; a PSMA CART cell embodiment is illustrated.

### Example 7

The purpose of this example was to evaluate the tumor killing capacity of different PSMA CART cell constructs.

FIG. 5 shows a graph demonstrating the tumor killing capacity of allogeneic PSMA CART cells comprising the TCR-α chain (TRAC) knockout (e.g., the construct used prior to the present disclosure), CD3δ knockout (D1), CD3ε knockout (E4), and CD3γ knockout (G4). The results illustrate that the PSMA E4 allogeneic CART cells have the best killing capacity. Target cells were PC3 cells, which is a human prostate cancer cell line.

These results were surprising and unexpected because it was not expected that the targeted disruption of one CD3 subunit would result in more potent allogeneic CART cells when compared to the targeted disruption of the other CD3 subunits. FIG. 5 demonstrates the unexpected finding that allo CART cells targeting CD3ε (e.g. CD3ε knockout (E4)) were more potent (i.e. kill tumor cells much faster) when compared to allo TCR-α chain (TRAC) knockout CART cells, allo CD3δ knockout (D1) CART cells, or allo CD3γ knockout (G4) CART cells.

### Example 8

The purpose of this example was to evaluate the effectiveness of the CRISPR-Cas methodology in effecting a knockout of the target gene. In particular,

FIGS. 6A-6D show CRISPR-Cas activity illustrated with the T7 endonuclease mismatch detection assay (T7E1). FIG. 6A show a representative gel electrophoresis image of T7E1-treated PCR products amplified from the sites of three different CRISPR-Cas C2TA (CIITA) gene using three different gRNA. FIGS. 6B-7D shows electropherograms generated by Agilent Bioanalyzer electropherogram of the T7E1 endonuclease assay demonstrating the CRISPR-Cas editing efficiency.

In addition, FIGS. 7A-D show Agilent Bioanalyzer electropherograms and gel electrophoresis of control and T7E1 treated PCR illustrating C2TA (CIITA) CRISPR editing efficiency result.

### Example 9

The purpose of this example was to determine the viability of different cell types.

A mixed lymphocyte assay (MLA) was conducted. FIG. 8 shows a graph illustrating the result of mixed lymphocyte reaction (MLR) assay with Allo Cells alone, T cells (second donor) alone, Allo Cells in co-culture, and T cells (second donor) in co-culture. In particular, recipient's T cells (T cells from a different donor) were co-cultured with allogeneic CART cells for 14 days, and proliferation of T cells were analyzed.

The results demonstrate the viability of control T cells (2^{nd} donor), allogeneic PSMA CART cells alone or in co-culture, and show that “recipient's” T cells did not react (no proliferation) to the presence of allogeneic cells. Therefore, FIG. 8 shows that T cells from a 2^{nd} (irrelevant) donor did not proliferate in response to allogeneic PSMA CART cells in co-culture despite the presence of an HLA mismatch. Allogeneic CART comprises PSMA CART and CRISPR edited TRAC/B2M/C2TA gRNAs. Accordingly, allogeneic CART cells of the present invention will have a window of opportunity to kill tumor cells while being undetected by the recipient's immune system (i.e. T cells).

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other publicly available documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A modified immune cell comprising:
(a) an insertion and/or deletion in one or more gene loci each encoding an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain), wherein the insertion and/or deletion is capable of downregulating gene expression of the one or more endogenous immune genes; and
(b) an exogenous nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen; and optionally
(c) further comprising a dominant negative receptor, a switch receptor, a chemokine, a chemokine receptor, a cytokine, a cytokine receptor, IL-7, IL-7R, IL-15, IL-15R, IL-21, IL-18, CCL21, CCL19, or a combination thereof.

2. The modified immune cell of claim 1, wherein the insertion and/or deletion is capable of downregulating the gene expression of:
(a) a T cell receptor subunit selected from CD3δ, CD3ε, and/or CD3γ;
(b) a HLA class I molecule selected from B2M, TAP1, TAP2, TAPBP, and/or NLRC5; and
(c) a HLA class II molecule selected from HLA-DM, RFX5, RFXANK, RFXAP, and/or invariant chain (Ii Chain).

3. The modified immune cell of claim 2, wherein:
(a) the insertion and/or deletion is capable of downregulating:
(i) the gene expression of CD3δ, and
(ii) the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof; or
(b) the insertion and/or deletion is capable of downregulating:
(i) the gene expression of CD3ε, and
(ii) the gene expression of a HLA molecule selected from the group consisting of B2M, TAP1, TAP2, TAPBP, NLRC5, CIITA, HLA-DM, RFX5, RFXANK, RFXAP, invariant chain (Ii Chain), and a combination thereof; or
(c) the insertion and/or deletion is capable of downregulating:
(i) the gene expression of CD3γ, and
(ii) the gene expression of a HLA molecule selected from the group consisting of B2M, TAP1, TAP2, TAPBP, NLRC5, CIITA, HLA-DM, RFX5, RFXANK, RFXAP, invariant chain (Ii Chain), and a combination thereof.

4. The modified immune cell of claim 1, wherein the insertion and/or deletion is capable of downregulating the gene expression of:
(I) any one of the following:
(a) CD3ε, B2M, and CIITA;
(b) CD3ε, B2M, and RFX5;
(c) CD3ε, B2M, and RFXAP;
(d) CD3ε, B2M, and RFXANK;
(e) CD3ε, B2M, and HLA-DM;
(f) CD3ε, B2M, and Ii chain;
(g) CD3ε, TAP1, and CIITA;
(h) CD3ε, TAP1, and RFX5;
(i) CD3ε, TAP1, and RFXAP;
(j) CD3ε, TAP1, and RFXANK;
(k) CD3ε, TAP1, and HLA-DM;
(l) CD3ε, TAP1, and Ii chain;
(m) CD3ε, TAP2, and CIITA;
(n) CD3ε, TAP2, and RFX5;
(o) CD3ε, TAP2, and RFXAP;
(p) CD3ε, TAP2, and RFXANK;
(q) CD3ε, TAP2, and HLA-DM;
(r) CD3ε, TAP2, and Ii chain;
(s) CD3ε, NLRC5, and CIITA;
(t) CD3ε, NLRC5, and RFX5;
(u) CD3ε, NLRC5, and RFXAP;
(v) CD3ε, NLRC5, and RFXANK;
(w) CD3ε, NLRC5, and HLA-DM;
(x) CD3ε, NLRC5, and Ii chain;
(y) CD3ε, TAPBP, and CIITA;
(z) CD3ε, TAPBP, and RFX5;
(aa) CD3ε, TAPBP, and RFXAP;
(bb) CD3ε, TAPBP, and RFXANK;
(cc) CD3ε, TAPBP, and HLA-DM; or
(dd) CD3ε, TAPBP, and Ii chain; or
(II) any one of the following:
(a) CD3δ, B2M, and CIITA;
(b) CD3δ, B2M, and RFX5;
(c) CD3δ, B2M, and RFXAP;
(d) CD3δ, B2M, and RFXANK;
(e) CD3δ, B2M, and HLA-DM;
(f) CD3δ, B2M, and Ii chain;
(g) CD3δ, TAP1, and CIITA;
(h) CD3δ, TAP1, and RFX5;
(i) CD3δ, TAP1, and RFXAP;
(j) CD3δ, TAP1, and RFXANK;
(k) CD3δ, TAP1, and HLA-DM;
(l) CD3δ, TAP1, and Ii chain;
(m) CD3δ, TAP2, and CIITA;
(n) CD3δ, TAP2, and RFX5;
(o) CD3δ, TAP2, and RFXAP;
(p) CD3δ, TAP2, and RFXANK;
(q) CD3δ, TAP2, and HLA-DM;
(r) CD3δ, TAP2, and Ii chain;
(s) CD3δ, NLRC5, and CIITA;
(t) CD3δ, NLRC5, and RFX5;
(u) CD3δ, NLRC5, and RFXAP;
(v) CD3δ, NLRC5, and RFXANK;
(w) CD3δ, NLRC5, and HLA-DM;
(x) CD3δ, NLRC5, and Ii chain;
(y) CD3δ, TAPBP, and CIITA;
(z) CD3δ, TAPBP, and RFX5;
(aa) CD3δ, TAPBP, and RFXAP;
(bb) CD3δ, TAPBP, and RFXANK;
(cc) CD3δ, TAPBP, and HLA-DM; or
(dd) CD3δ, TAPBP, and Ii chain; or
(III) any one of the following:
(a) CD3γ, B2M, and CIITA;
(b) CD3γ, B2M, and RFX5;
(c) CD3γ, B2M, and RFXAP;
(d) CD3γ, B2M, and RFXANK;
(e) CD3γ, B2M, and HLA-DM;
(f) CD3γ, B2M, and Ii chain;
(g) CD3γ, TAP1, and CIITA;
(h) CD3γ, TAP1, and RFX5;
(i) CD3γ, TAP1, and RFXAP;
(j) CD3γ, TAP1, and RFXANK;
(k) CD3γ, TAP1, and HLA-DM;
(l) CD3γ, TAP1, and Ii chain;
(m) CD3γ, TAP2, and CIITA;
(n) CD3γ, TAP2, and RFX5;
(o) CD3γ, TAP2, and RFXAP;
(p) CD3γ, TAP2, and RFXANK;
(q) CD3γ, TAP2, and HLA-DM;
(r) CD3γ, TAP2, and Ii chain;
(s) CD3γ, NLRC5, and CIITA;
(t) CD3γ, NLRC5, and RFX5;
(u) CD3γ, NLRC5, and RFXAP;
(v) CD3γ, NLRC5, and RFXANK;
(w) CD3γ, NLRC5, and HLA-DM;
(x) CD3γ, NLRC5, and Ii chain;
(y) CD3γ, TAPBP, and CIITA;
(z) CD3γ, TAPBP, and RFX5;
(aa) CD3γ, TAPBP, and RFXAP;
(bb) CD3γ, TAPBP, and RFXANK;
(cc) CD3γ, TAPBP, and HLA-DM; or
(dd) CD3γ, TAPBP, and Ii chain.

5. The modified immune cell of claim 1, wherein:
(a) the modified immune cell is selected from the group consisting of a T cell, a natural killer cell (NK cell), a natural killer T cell, a lymphoid progenitor cell, a hematopoietic stem cell, a stem cell, a macrophage, and a dendritic cell; and/or
(b) the modified immune cell is a CD4+ T cell or a CD8+ T cell; and/or
(c) the modified immune cell is an allogeneic T cell or autologous human T cell.

6. The modified immune cell of claim 1, wherein the insertion and/or deletion is the result of gene editing selected from the group consisting of:
(a) a CRISPR-associated (Cas) (CRISPR-CAs) endonuclease system and a guide RNA;
(b) a TALEN gene editing system, a zinc finger nuclease (ZFN) gene editing system, a meganuclease gene editing system, or a mega-TALEN gene editing system; and
(c) a gene silencing system selected from antisense RNA, antigomer RNA, RNAi, siRNA, or shRNA.

7. The modified immune cell of claim 6, wherein:
(a) the Cas endonuclease comprises Cas3, Cas4, Cas8a, Cas8b, Cas9, Cas10, Cas10d, Cas12a, Cas12b, Cas12d, Cas12e, Cas12f, Cas12g, Cas12h, Cas12i, Cas13, Cas14, CasX, Cse1, Csy1, Csn2, Cpf1, C2c1, Csm2, Cmr5, Fok1, S. pyogenes Cas9, Staphylococcus aureus Cas9, MAD7, or any combination thereof; or
(b) the CRISPR-Cas system comprises an pAd5/F35-CRISPR vector; or
(c) the guide RNA comprises a guide sequence that is complementary with a sequence within the one or more gene loci each encoding the immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain).

8. The modified immune cell of claim 7, wherein:
(a) the guide RNA is complementary with a sequence within: (1) one or more exons of CD3δ, CD3ε, or CD3γ, or (2) exon 1 of CD3δ, CD3ε, or CD3γ; or
(b) the sequence is within the CD3δ gene locus and the guide RNA comprises a nucleic acid sequence encoded by SEQ ID NO: 53; or
(c) the sequence is within the CD3ε gene locus and the guide RNA comprises a nucleic acid sequence encoded by SEQ ID NO: 52; or
(d) the sequence is within the CD3γ gene locus and the guide RNA comprises a nucleic acid sequence encoded by SEQ ID NO: 54; or
(e) the sequence is within the B2M gene locus and the guide RNA comprises a nucleic acid sequence encoded by SEQ ID NO: 55; or
the sequence is within the CIITA gene locus and the guide RNA comprises a nucleic acid sequence encoded by SEQ ID NO: 61; or
(g) the sequence is within the TAP1 gene locus and the guide RNA comprises a nucleic acid sequence encoded by SEQ ID NO: 56; or
(h) the sequence is within the TAP2 gene locus and the guide RNA comprises a nucleic acid sequence encoded by SEQ ID NO: 57; or
(i) the sequence is within the TAPBP gene locus and the guide RNA comprises a nucleic acid sequence encoded by SEQ ID NO: 58, SEQ ID NO: 59, or a combination thereof; or
(j) the sequence is within the NLRC5 gene locus and the guide RNA comprises a nucleic acid sequence encoded by SEQ ID NO: 60; or
(k) the sequence is within the HLA-DM gene locus and the guide RNA comprises a nucleic acid sequence encoded by SEQ ID NO: 62; or
(l) the sequence is within the RFX5 gene locus and the guide RNA comprises a nucleic acid sequence encoded by SEQ ID NO: 63, SEQ ID NO: 64, or a combination thereof; or
(m) the sequence is within the RFXANK gene locus and the guide RNA comprises a nucleic acid sequence encoded by SEQ ID NO: 65; or
(n) the sequence is within the RFXAP gene locus and the guide RNA comprises a nucleic acid sequence encoded by SEQ ID NO: 66; or
(o) the sequence is within the Ii Chain gene locus and the guide RNA comprises a nucleic acid sequence encoded by SEQ ID NO: 67, SEQ ID NO: 68, or a combination.

9. The modified immune cell of claim 1, wherein:
(a) the immune cell exerts a reduced immune response in a subject when the modified immune cell is administered to the subject, as compared to the immune response exerted by an unmodified immune cell administered to the same subject;
(b) the immune cell exerts a reduced immune response in a subject when the modified immune cell is administered to the subject, as compared to the immune response exerted by an immune cell comprising an insertion and/or deletion capable of downregulating the gene expression of TRAC, B2M, and CIITA, and optionally wherein the immune response is a graft-versus-host disease (GvHD) response, and further optionally wherein the reduced GvHD response is elicited against an HLA-I mismatched cell or against an HLA-II mismatched cell.

10. The modified immune cell of claim 9, wherein:
(a) the GvHD response is reduced by about 10% or more, about 20% or more, about 30% or more, about 40% or more, about 50% or more, about 60% or more, about 70% or more, about 80% or more, about 90% or more, or about 95% or more; or
(b) the GvHD response is reduced by about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 20-fold or more, about 30-fold or more, about 50-fold or more, about 100-fold or more, about 150-fold or more, or about 200-fold or more; and/or
(c) the reduced GvHD response by the modified immune cell is compared to an equivalent immune cell without the deletion and/or insertion in one or more gene loci, or an immune cell comprising the deletion and/or insertion in TRAC, B2M, and CIITA.

11. The modified immune cell of claim 1, wherein the exogenous nucleic acid encodes a chimeric antigen receptor (CAR), and wherein the CAR comprises an antigen binding domain, a hinge domain, a transmembrane domain, a costimulatory signaling domain, and an intracellular signaling domain.

12. The modified immune cell of claim 11, wherein:
(a) the antigen-binding domain comprises a full length antibody or an antigen-binding fragment thereof, a Fab, a F(ab)₂, a monospecific Fab₂, a bispecific Fab₂, a trispecific Fab₂, a single-chain variable fragment (scFv), a diabody, a triabody, a minibody, a V-NAR, or a VhH; and/or
(b) the transmembrane domain is selected from an artificial hydrophobic sequence, a transmembrane domain of a type I transmembrane protein, an alpha, beta, or zeta chain of a T cell receptor, CD28, CD3 epsilon, CD45, CD4, CD2, CD5, CD8, CD9, CD16, CD22, CD33, CD37, CD64, CD80, CD86, OX40 (CD134), 4-1BB (CD137), ICOS (CD278), CD154, CD357 (GITR), Toll-like receptor 1 (TLR1), TLR2, TLR3, TLR4, TLR5, TLR6, TLR7, TLR8, TLR9, and a transmembrane domain derived from a killer immunoglobulin-like receptor (KIR); and/or
(c) the costimulatory domain comprises one or more of a costimulatory domain of a protein selected from the group consisting of proteins in the TNFR superfamily, CD28, 4-1BB (CD137), OX40 (CD134), PD-1, CD7, LIGHT, CD83L, DAP10, DAP12, CD27, CD2, CD5, ICAM-1, LFA-1, Lck, TNFR-I, TNFR-II, Fas, CD30, CD40, ICOS (CD278), NKG2C, B7-H3 (CD276), and an intracellular domain derived from a killer immunoglobulin-like receptor (KIR), or a variant thereof; and/or
(d) the intracellular signaling domain comprises an intracellular domain selected from the group consisting of cytoplasmic signaling domains of a human CD2, CD3 zeta chain (CD3), FcγRIII, FcsRI, a cytoplasmic tail of an Fc receptor, an immunoreceptor tyrosine-based activation motif (ITAM) bearing cytoplasmic receptor, TCR zeta, FcR gamma, CD3 gamma, CD3 delta, CD3 epsilon, CD5, CD22, CD79a, CD79b, and CD66d, or a variant thereof; and/or
(e) the antigen binding domain targets a tumor antigen is:
(i) associated with a hematologic malignancy;
(ii) associated with a solid tumor; and/or
(iii) selected from the group consisting of ROR1, mesothelin, c-Met, PSMA, PSCA, Folate receptor alpha, Folate receptor beta, EGFR, EGFRvIII, GPC2, GPC2, Mucin 1 (MUC1), Tn antigen ((Tn Ag) or (GalNAca-Ser/Thr)), TnMUC1, GDNF family receptor alpha-4 (GFRa4), fibroblast activation protein (FAP), and Interleukin-13 receptor subunit alpha-2 (IL-13Ra2 or CD213A2); and/or
(f) the intracellular signaling domain comprises a human CD3 zeta chain (CD3ζ); and/or
(g) the CAR comprises:
(i) a PSMA antigen binding domain, a CD2 costimulatory domain, and a CD3 zeta intracellular signaling domain;
(ii) a mesothelin antigen binding domain, a 4-1BB costimulatory domain, and a CD3 zeta signaling domain; or
(iii) a TnMUC1 antigen binding domain, a CD2 costimulatory domain, and a CD3 zeta signaling domain.

13. The modified immune cell of claim 1, wherein:
(a) the switch receptor comprises an extracellular domain of a signaling protein associated with a negative signal, a transmembrane domain, and an intracellular domain of a signaling protein associated with a positive signal; and/or
(b) the dominant negative receptor comprises:
(i) a truncated variant of a wild-type protein associated with a negative signal;
(ii) a variant of a wild-type protein associated with a negative signal comprising an extracellular domain, a transmembrane domain, and substantially lacking an intracellular signaling domain; or
(iii) an extracellular domain of a signaling protein associated with a negative signal, and a transmembrane domain.

14. The modified immune cell of claim 13, wherein:
(a) the protein associated with the negative signal is selected from the group consisting of CTLA4, PD-1, TGFβRII, BTLA, VSIG3, VSIG8, and TIM-3; and/or
(b) the protein associated with the positive signal is selected from the group consisting of CD28, 4-1BB, IL12Rβ1, IL12Rβ2, CD2, ICOS, and CD27; and/or
(c) the switch receptor is selected from the group consisting of PD-1-CD28, PD-1^{A132L}-CD28, PD-1-CD27, PD-1^{A132L}-CD27, PD-1-4-1BB, PD-1^{A132L}-4-1BB, PD-1-ICOS, PD-1^{A132L}-ICOS, PD-1-IL12Rβ1, PD-1^{A132L}-IL12Rβ1, PD-1-IL12Rβ2, PD-1^{A132L}-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2; and/or
(d) the dominant negative receptor is PD1, VSIG3, VISG8, or TGFPR dominant negative receptor; and/or
(e) the transmembrane domain is:
(i) selected from a transmembrane domain of a protein selected from the group consisting of CTLA4, PD-1, VSIG3, VSIG8, TGFβRII, BTLA, TIM-3, CD28, 4-1BB, IL12Rβ1, IL12Rβ2, CD2, ICOS, and CD27; or
(ii) selected from the transmembrane of the protein associated with a negative signal or the transmembrane domain of the protein associated with the negative signal.

15. An isolated modified T cell, comprising at least one functionally impaired polypeptide selected from the group consisting of CD3δ, CD3ε, CD3γ, B2M, C2TA, TAP1, TAP2, TAPBP, NLRC5, HLA-DM, RFX5, RFXANK, RFXAP, and invariant chain (Ii Chain);
wherein the modified T cell comprising the functionally impaired polypeptide exhibits at least one of:
(i) reduced T cell receptor expression as compared to an unmodified T cell;
(ii) reduced expression of the impaired polypeptide;
(iii) complete absence of the T cell receptor complex surface expression; and
(iv) reduced or insufficient T cell receptor cross-linking.

16. The isolated modified T cell of claim 15, wherein:
(a) the modified T cell exerts a reduced immune response in a subject when the modified T cell is administered to the subject, as compared to the immune response exerted by an unmodified T cell administered to the same subject; and/or
(b) the modified T cell comprises two or more functionally impaired polypeptides, and wherein the second impaired polypeptide is T-cell receptor alpha chain (TRAC); and/or
(c) the modified T cell comprises:
(i) three or more functionally impaired polypeptides selected from TRAC, CD3δ, CD3ε, CD3γ, B2M, C2TA, TAP1, TAP2, TAPBP, NLRC5, HLA-DM, RFX5, RFXANK, RFXAP, or Ii chain;
(ii) two functionally impaired polypeptides selected from CD3α, CD3δ, CD3ε, CD3γ, B2M, C2TA, TAP1, TAP2, TAPBP, NLRC5, HLA-DM, RFX5, RFXANK, RFXAP, or Ii chain;
(iii) three functionally impaired polypeptides selected from CD3α, CD3δ, CD3ε, CD3γ, B2M, C2TA, TAP1, TAP2, TAPBP, NLRC5, HLA-DM, RFX5, RFXANK, RFXAP, or Ii chain;
(iv) a functionally impaired polypeptide selected from the group consisting of CD3δ, CD3ε, and CD3γ, and at least one functionally impaired polypeptide selected from TRAC, B2M, C2TA, TAP1, TAP2, TAPBP, NLRC5, HLA-DM, RFX5, RFXANK, RFXAP, or Ii Chain; or
(v) a functionally impaired polypeptide selected from the group consisting of CD3δ, CD3ε, and CD3γ, and a functionally impaired polypeptide selected from TRAC, B2M, C2TA, TAP1, TAP2, TAPBP, NLRC5, HLA-DM, RFX5, RFXANK, RFXAP, or Ii Chain; and/or
(d) the modified T cell comprises:
(i) functionally impaired CD3δ and at least one functionally impaired polypeptide selected from TRAC, B2M, C2TA, TAP1, TAP2, TAPBP, NLRC5, HLA-DM, RFX5, RFXANK, RFXAP, or Ii chain;
(ii) functionally impaired CD3ε and at least one functionally impaired polypeptide selected from TRAC, B2M, C2TA, TAP1, TAP2, TAPBP, NLRC5, HLA-DM, RFX5, RFXANK, RFXAP, or Ii chain; or
(iii) functionally impaired CD3γ and at least one functionally impaired polypeptide selected from TRAC, B2M, C2TA, TAP1, TAP2, TAPBP, NLRC5, HLA-DM, RFX5, RFXANK, RFXAP, or Ii chain; and/or
(e) the modified T cell comprises two or more functionally impaired polypeptides selected from TRAC, B2M, C2TA, TAP1, TAP2, TAPBP, NLRC5, HLA-DM, RFX5, RFXANK, RFXAP, or Ii chain; and/or
(f) the modified T cell:
(i) has a reduced expression of TRAC, CD3δ, CD3ε, CD3γ, B2M, C2TA, TAP1, TAP2, TAPBP, NLRC5, HLA-DM, RFX5, RFXANK, RFXAP, Ii chain, or any combination thereof, or
(ii) does not express , CD3δ, CD3ε, CD3γ, TRAC, B2M, C2TA, TAP1, TAP2, TAPBP, NLRC5, HLA-DM, RFX5, RFXANK, RFXAP, Ii chain, or any combination thereof; and/or
(g) the modified T cell further comprises a functionally impaired polypeptide selected from TRAC, B2M, and C2TA; and/or
(h) the modified T cell has a reduced expression of TRAC, B2M, or C2TA or does not express TRAC, B2M, or C2TA; and/or
(i) modification of CD3δ, CD3ε, and/or CD3γ leads to an impaired TCR/CD3 complex function; and/or
(j) CD3δ, CD3ε, or CD3γ is modified by targeting one or more exons of CD3δ, CD3ε, or CD3γ, optionally exon 1 of CD3δ, CD3ε, or CD3γ.

17. A method for generating a modified immune cell comprising:
(a) introducing into the immune cell one or more nucleic acids capable of downregulating gene expression of one or more endogenous immune genes encoding an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain);
(b) introducing into the immune cell an exogenous nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen; and
(c) expanding the modified immune cell to generate a population of T cells; and optionally
(d) further comprising introducing into the immune cell an exogenous nucleic acid encoding a dominant negative receptor, a switch receptor, or a combination thereof.

18. The method of claim 17, wherein:
(a) wherein the one or more nucleic acids are capable of downregulating the gene expression of:
(i) a T cell receptor subunit selected from CD3δ, CD3ε, or CD3γ;
(ii) a HLA class I molecule selected from B2M, TAP1, TAP2, TAPBP, or NLRC5; and
(iii) a HLA class II molecule selected from HLA-DM, RFX5, RFXANK, RFXAP, or invariant chain (Ii Chain); and/or
(b) wherein the one or more nucleic acids are capable of downregulating the gene expression of CD3δ, and the gene expression of a HLA molecule selected from the group consisting of B2M, TAP1, TAP2, TAPBP, NLRC5, HLA-DM, RFX5, RFXANK, RFXAP, invariant chain (Ii Chain), and a combination thereof; and/or
(c) wherein the one or more nucleic acids are capable of downregulating the gene expression of CD3ε, and a HLA molecule selected from the group consisting of B2M, TAP1, TAP2, TAPBP, NLRC5, CIITA, HLA-DM, RFX5, RFXANK, RFXAP, invariant chain (Ii Chain), and a combination thereof; and/or
(d) wherein the one or more nucleic acids are capable of downregulating the gene expression of CD3γ, and a HLA molecule selected from the group consisting of B2M, TAP1, TAP2, TAPBP, NLRC5, CIITA, HLA-DM, RFX5, RFXANK, RFXAP, invariant chain (Ii Chain), and a combination thereof.

19. The method of claim 17, wherein the one or more nucleic acids are capable of downregulating the gene expression of:
(I) any one of the following:
(a) CD3ε, B2M, and CIITA;
(b) CD3ε, B2M, and RFX5;
(c) CD3ε, B2M, and RFXAP;
(d) CD3ε, B2M, and RFXANK;
(e) CD3ε, B2M, and HLA-DM;
(f) CD3ε, B2M, and Ii chain;
(g) CD3ε, TAP1 and CIITA;
(h) CD3ε, TAP1, and RFX5;
(i) CD3ε, TAP1, and RFXAP;
(j) CD3ε, TAP1, and RFXANK;
(k) CD3ε, TAP1, and HLA-DM;
(l) CD3ε, TAP1, and Ii chain;
(m) CD3ε, TAP2, and CIITA;
(n) CD3ε, TAP2, and RFX5;
(o) CD3ε, TAP2, and RFXAP;
(p) CD3ε, TAP2, and RFXANK;
(q) CD3ε, TAP2, and HLA-DM;
(r) CD3ε, TAP2, and Ii chain;
(s) CD3ε, NLRC5, and CIITA;
(t) CD3ε, NLRC5, and RFX5;
(u) CD3ε, NLRC5, and RFXAP;
(v) CD3ε, NLRC5, and RFXANK;
(w) CD3ε, NLRC5, and HLA-DM;
(x) CD3ε, NLRC5, and Ii chain;
(y) CD3ε, TAPBP, and CIITA;
(z) CD3ε, TAPBP, and RFX5;
(aa) CD3ε, TAPBP, and RFXAP;
(bb) CD3ε, TAPBP, and RFXANK;
(cc) CD3ε, TAPBP, and HLA-DM; or
(dd) CD3ε, TAPBP, and Ii chain; or
(II) any one of the following:
(a) CD3δ, B2M, and CIITA;
(b) CD3δ, B2M, and RFX5;
(c) CD3δ, B2M, and RFXAP;
(d) CD3δ, B2M, and RFXANK;
(e) CD3δ, B2M, and HLA-DM;
(f) CD3δ, B2M, and Ii chain;
(g) CD3δ, TAP1, and CIITA;
(h) CD3δ, TAP1, and RFX5;
(i) CD3δ, TAP1, and RFXAP;
(j) CD3δ, TAP1, and RFXANK;
(k) CD3δ, TAP1, and HLA-DM;
(l) CD3δ, TAP1, and Ii chain;
(m) CD3δ, TAP2, and CIITA;
(n) CD3δ, TAP2, and RFX5;
(o) CD3δ, TAP2, and RFXAP;
(p) CD3δ, TAP2, and RFXANK;
(q) CD3δ, TAP2, and HLA-DM;
(r) CD3δ, TAP2, and Ii chain;
(s) CD3δ, NLRC5, and CIITA;
(t) CD3δ, NLRC5, and RFX5;
(u) CD3δ, NLRC5, and RFXAP;
(v) CD3δ, NLRC5, and RFXANK;
(w) CD3δ, NLRC5, and HLA-DM;
(x) CD3δ, NLRC5, and Ii chain;
(y) CD3δ, TAPBP, and CIITA;
(z) CD3δ, TAPBP, and RFX5;
(aa) CD3δ, TAPBP, and RFXAP;
(bb) CD3δ, TAPBP, and RFXANK;
(cc) CD3δ, TAPBP, and HLA-DM; or
(dd) CD3δ, TAPBP, and Ii chain; or
(III) any one of the following:
(a) CD3γ, B2M, and CIITA;
(b) CD3γ, B2M, and RFX5;
(c) CD3γ, B2M, and RFXAP;
(d) CD3γ, B2M, and RFXANK;
(e) CD3γ, B2M, and HLA-DM;
(f) CD3γ, B2M, and Ii chain;
(g) CD3γ, TAP1, and CIITA;
(h) CD3γ, TAP1, and RFX5;
(i) CD3γ, TAP1, and RFXAP;
(j) CD3γ, TAP1, and RFXANK;
(k) CD3γ, TAP1, and HLA-DM;
(l) CD3γ, TAP1, and Ii chain;
(m) CD3γ, TAP2, and CIITA;
(n) CD3γ, TAP2, and RFX5;
(o) CD3γ, TAP2, and RFXAP;
(p) CD3γ, TAP2, and RFXANK;
(q) CD3γ, TAP2, and HLA-DM;
(r) CD3γ, TAP2, and Ii chain;
(s) CD3γ, NLRC5, and CIITA;
(t) CD3γ, NLRC5, and RFX5;
(u) CD3γ, NLRC5, and RFXAP;
(v) CD3γ, NLRC5, and RFXANK;
(w) CD3γ, NLRC5, and HLA-DM;
(x) CD3γ, NLRC5, and Ii chain;
(y) CD3γ, TAPBP, and CIITA;
(z) CD3γ, TAPBP, and RFX5;
(aa) CD3γ, TAPBP, and RFXAP;
(bb) CD3γ, TAPBP, and RFXANK;
(cc) CD3γ, TAPBP, and HLA-DM; or
(dd) CD3γ, TAPBP, and Ii chain.

20. The method of claim 17, wherein:
(a) the immune cell is selected from the group consisting of a T cell, a natural killer cell (NK cell), a natural killer T cell, a lymphoid progenitor cell, a hematopoietic stem cell, a stem cell, a macrophage, and a dendritic cell; and/or
(b) the immune cell is a CD4+ T cell or a CD8+ T cell; and/or
(c) the immune cell is an allogeneic T cell or autologous T cell.

21. The method of claim 17, wherein:
(a) the nucleic acids are introduced into the immune cell by viral transduction, wherein the viral transduction comprises contacting the immune cell with a viral vector comprising the one or more nucleic acids; and/or
(b) the nucleic acids are introduced into the immune cell by viral transduction, wherein the viral transduction comprises contacting the immune cell with a viral vector comprising the one or more nucleic acids, and further wherein the viral vector is selected from the group consisting of a retroviral vector, sendai viral vectors, adenoviral vectors, adeno-associated virus vectors, and lentiviral vectors; and/or
(c) each of the one or more nucleic acids capable of downregulating expression comprises a gene editing system selected from the group consisting of:
(i) a CRISPR-associated (Cas) (CRISPR-CAs) endonuclease system and a guide RNA;
(ii) a TALEN gene editing system, a zinc finger nuclease (ZFN) gene editing system, a meganuclease gene editing system, or a mega-TALEN gene editing system; and
(iii) a gene silencing system selected from antisense RNA, antigomer RNA, RNAi, siRNA, or shRNA.

22. The method of claim 21, wherein :
(a) the Cas endonuclease comprises Cas3, Cas4, Cas8a, Cas8b, Cas9, Cas10, Cas10d, Cas12a, Cas12b, Cas12d, Cas12e, Cas12f, Cas12g, Cas12h, Cas12i, Cas13, Cas14, CasX, Cse1, Csy1, Csn2, Cpf1, C2c1, Csm2, Cmr5, Fok1, S. pyogenes Cas9, Staphylococcus aureus Cas9, MAD7, or any combination thereof ; and/or
(b) the CRISPR-Cas system comprises an pAd5/F35-CRISPR vector; and/or
(c) the guide RNA comprises a guide sequence that is complementary with a sequence within the one or more gene loci each encoding the immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain); and/or
(d) the guide RNA is complementary with a sequence within: (1) one or more exons of CD3δ, CD3ε, or CD3γ, or (2) exon 1 of CD3δ, CD3ε, or CD3γ; and/or
(e) the sequence is within the CD3δ gene locus and the guide RNA comprises a nucleic acid sequence encoded by SEQ ID NO: 53;
(f) the sequence is within the CD3ε gene locus and the guide RNA comprises a nucleic acid sequence encoded by SEQ ID NO: 52;
(g) the sequence is within the CD3γ gene locus and the guide RNA comprises a nucleic acid sequence encoded by SEQ ID NO: 54;
(h) the sequence is within the B2M gene locus and the guide RNA comprises a nucleic acid sequence encoded by SEQ ID NO: 55;
(i) the sequence is within the CIITA gene locus and the guide RNA comprises a nucleic acid sequence encoded by SEQ ID NO: 61;
(j) the sequence is within the TAP1 gene locus and the guide RNA comprises a nucleic acid sequence encoded by SEQ ID NO: 56;
(k) the sequence is within the TAP2 gene locus and the guide RNA comprises a nucleic acid sequence encoded by SEQ ID NO: 57;
(l) the sequence is within the TAPBP gene locus and the guide RNA comprises a nucleic acid sequence encoded by SEQ ID NO: 58, SEQ ID NO: 59, or a combination thereof;
(m) the sequence is within the NLRC5 gene locus and the guide RNA comprises a nucleic acid sequence encoded by SEQ ID NO: 60;
(n) the sequence is within the HLA-DM gene locus and the guide RNA comprises a nucleic acid sequence encoded by SEQ ID NO: 62;
(o) the sequence is within the RFX5 gene locus and the guide RNA comprises a nucleic acid sequence encoded by SEQ ID NO: 63, SEQ ID NO: 64, or a combination thereof;
(p) the sequence is within the RFXANK gene locus and the guide RNA comprises a nucleic acid sequence encoded by SEQ ID NO: 65;
(q) the sequence is within the RFXAP gene locus and the guide RNA comprises a nucleic acid sequence encoded by SEQ ID NO: 66 or
(r) the sequence is within the Ii Chain gene locus and the guide RNA comprises a nucleic acid sequence encoded by SEQ ID NO: 67, SEQ ID NO: 68, or a combination thereof.

23. The method of claim 17, wherein:
(a) the immune cell exerts a reduced immune response in a subject when the immune cell is administered to the subject, as compared to the immune response exerted by an unmodified immune cell administered to the same subject; and/or
(b) the immune cell exerts a reduced immune response in a subject when the immune cell is administered to the subject, as compared to the immune response exerted by an immune cell comprising one or more nucleic acids capable of downregulating the gene expression of TRAC, B2M, and CIITA; and/or
(c) the immune cell exerts a reduced immune response in a subject when the immune cell is administered to the subject, wherein the immune response is a graft-versus-host disease (GvHD) response; and/or
(d) the immune cell exerts a reduced immune response in a subject when the immune cell is administered to the subject, wherein the immune response is a graft-versus-host disease (GvHD) response, and further wherein the reduced GvHD response is elicited against an HLA-I mismatched cell or against an HLA-II mismatched cell; and/or
(e) the immune cell exerts a reduced immune response in a subject when the immune cell is administered to the subject, wherein the immune response is a graft-versus-host disease (GvHD) response, wherein the GvHD response is:
(i) reduced by about 10% or more, about 20% or more, about 30% or more, about 40% or more, about 50% or more, about 60% or more, about 70% or more, about 80% or more, about 90% or more, or about 95% or more; or
(ii) reduced by about 1-fold or more, about 2-fold or more, about 3-fold or more, about 4-fold or more, about 5-fold or more, about 6-fold or more, about 7-fold or more, about 8-fold or more, about 9-fold or more, about 10-fold or more, about 20-fold or more, about 30-fold or more, about 50-fold or more, about 100-fold or more, about 150-fold or more, or about 200-fold or more; and/or
(f) the immune cell exerts a reduced immune response in a subject when the immune cell is administered to the subject, wherein the immune response is a graft-versus-host disease (GvHD) response, and further wherein the reduced GvHD response by the modified immune cell is compared to an equivalent immune cell without the deletion and/or insertion in one or more gene loci, or an immune cell comprising the deletion and/or insertion in TRAC, B2M, and CIITA.

24. The method of claim 17, wherein:
(a) the exogenous nucleic acid encodes a chimeric antigen receptor (CAR), and wherein the CAR comprises an antigen binding domain, a hinge domain, a transmembrane domain, a costimulatory signaling domain, and an intracellular signaling domain; and/or
(b) the exogenous nucleic acid encodes a chimeric antigen receptor (CAR), and wherein the CAR comprises an antigen binding domain, a hinge domain, a transmembrane domain, a costimulatory signaling domain, and an intracellular signaling domain, and further wherein the antigen binding domain targets a tumor antigen:
(i) associated with a hematologic malignancy;
(ii) associated with a solid tumor; and/or
(iii) selected from the group consisting of ROR1, mesothelin, c-Met, PSMA, PSCA, Folate receptor alpha, Folate receptor beta, EGFR, EGFRvIII, GPC2, GPC2, Mucin 1 (MUC1), Tn antigen ((Tn Ag) or (GalNAca-Ser/Thr)), TnMUC1, GDNF family receptor alpha-4 (GFRa4), fibroblast activation protein (FAP), and Interleukin-13 receptor subunit alpha-2 (IL-13Ra2 or CD213A2); and/or
(c) the exogenous nucleic acid encodes a chimeric antigen receptor (CAR), and wherein the CAR comprises an antigen binding domain, a hinge domain, a transmembrane domain, a costimulatory signaling domain, and an intracellular signaling domain, and further wherein the CAR comprises:
(i) a PSMA antigen binding domain, a CD2 costimulatory domain, and a CD3 zeta intracellular signaling domain;
(ii) a mesothelin antigen binding domain, a 4-1BB costimulatory domain, and a CD3 zeta signaling domain; or
(iii) a TnMUC1 antigen binding domain, a CD2 costimulatory domain, and a CD3 zeta signaling domain; and/or
(d) the switch receptor comprises:
(i) an extracellular domain of a signaling protein associated with a negative signal selected from the group consisting of CTLA4, PD-1, VISG3, VSIG8, TGFβRII, BTLA, and TIM-3,
(ii) a transmembrane domain, and
(iii) an intracellular domain of a signaling protein associated with a positive signal selected from the group consisting of CD28, 4-1BB, IL12Rβ1, IL12Rβ2, CD2, ICOS, and CD27; and/or
(e) the dominant negative receptor comprises:
(i) a truncated variant of a wild-type protein associated with a negative signal,
(ii) a variant of a wild-type protein associated with a negative signal comprising an extracellular domain, a transmembrane domain, and substantially lacking an intracellular signaling domain; or
(iii) an extracellular domain of a signaling protein associated with a negative signal, and a transmembrane domain; and/or
(f) the switch receptor is selected from the group consisting of PD-1-CD28, PD-1^{A132L}-CD28, PD-1-CD27, PD-1^{A132L}-CD27, PD-1-4-1BB, PD-1^{A132L}-4-1BB, PD-1-ICOS, PD-1^{A132L}-ICOS, PD-1-IL12Rβ1, PD-1^{A132L}-IL12Rβ1, PD-1-IL12Rβ2, PD-1^{A132L}-IL12Rβ2, VSIG3-CD28, VSIG8-CD28, VSIG3-CD27, VSIG8-CD27, VSIG3-4-1BB, VSIG8-4-1BB, VSIG3-ICOS, VSIG8-ICOS, VSIG3-IL12Rβ1, VSIG8-IL12Rβ1, VSIG3-IL12Rβ2, VSIG8-IL12Rβ2, TGFβRII-CD27, TGFβRII-CD28, TGFβRII-4-1BB, TGFβRII-ICOS, TGFβRII-IL12Rβ1, and TGFβRII-IL12Rβ2; and/or
(g) the dominant negative receptor is PD1, VSIG3, VSIG8, or TGFPR dominant negative receptor; and/or
(h) the transmembrane domain is:
(i) selected from a transmembrane domain of a protein selected from the group consisting of CTLA4, PD-1, BTLA, TGFβRII, BTLA, TIM-3, CD28, 4-1BB, IL12Rβ1, IL12Rβ2, CD2, ICOS, and CD27; or
(ii) selected from the transmembrane of the protein associated with a negative signal or the transmembrane domain of the protein associated with the negative signal.

25. The method of claim 17:
(a) wherein expanding the modified immune cell comprises culturing the T cell with a factor selected from the group consisting of flt3-L, IL-1, IL-3, IL-2, IL-7, IL-15, IL-18, IL-21, TGFbeta, IL-10, and c-kit ligand; and/or
(b) further comprising introducing a polypeptide and/or a nucleic acid encoding Klf4, Oct3/4 and Sox2 in the immune cell to induce pluripotency of the immune cell; and/or
(c) wherein the immune cell is obtained from a blood sample, a whole blood sample, a peripheral blood mononuclear cell (PBMC) sample, or an apheresis sample; and/or
(d) wherein the immune cell is obtained from an apheresis sample and further wherein the apheresis sample is a cryopreserved sample; and/or
(e) wherein the immune cell is obtained from an apheresis sample and further wherein the apheresis sample is a fresh sample; and/or
(f) wherein the immune cell is obtained from a human subject.

26. A population of modified immune cells obtained from the method of claim 17.

27. A composition comprising the modified immune cell of claim 1, and a pharmaceutically acceptable carrier or excipient.

28. A method of treating a disease or condition associated with enhanced immunity in a subject comprising administering an effective amount of a composition to a subject in need thereof, wherein the composition comprises a modified immune cell comprising:
(a) an insertion and/or deletion in one or more gene loci each encoding an endogenous immune protein selected from the group consisting of CD3δ, CD3ε, CD3γ, B2M, CIITA, TAP1, TAP2, TAPBP, NLRC5, HLA-DM, RFX5, RFXANK, RFXAP, and invariant chain (Ii Chain), wherein the insertion and/or deletion is capable of downregulating gene expression of the one or more endogenous immune genes; and
(b) an exogenous nucleic acid encoding a chimeric antigen receptor (CAR), an engineered T cell receptor (TCR), a Killer cell immunoglobulin-like receptor (KIR), an antigen-binding polypeptide, a cell surface receptor ligand, or a tumor antigen; and optionally
(c) further comprising a dominant negative receptor, a switch receptor, a chemokine, a chemokine receptor, a cytokine, a cytokine receptor, IL-7, IL-7R, IL-15, IL-15R, IL-21, IL-18, CCL21, CCL19, or a combination thereof.

29. The method of claim 28, wherein:
(a) the condition is a cancer; and/or
(b) the condition is a cancer and further wherein the cancer is selected from the group consisting of breast cancer, prostate cancer, ovarian cancer, cervical cancer, skin cancer, pancreatic cancer, colorectal cancer, renal cancer, liver cancer, brain cancer, lymphoma, leukemia, lung cancer, and any combination thereof; and/or
(c) the condition is a cancer and the cancer is a solid tumor, or a hematologic malignancy.

30. A method of treating a cancer, comprising administering to a subject the composition of claim 27.

31. The method of claim 30, wherein the cancer is a solid tumor, or a hematologic malignancy.

32. A method for stimulating a T cell-mediated immune response to a target cell or tissue in a subject comprising administering to a subject an effective amount of the composition of claim 27.

33. A kit comprising the composition of claim 27, optionally comprising an instruction for use.
